(19) United States
(12) Patent Application Publication
(10) Pub. No.: US 2026/0124250 A1
(43) Pub. Date: May 7, 2026

(54) METHODS FOR SELECTING CULTURED CORNEAL ENDOTHELIAL CELLS

(71) Applicants: Universiteit Maastricht, Maastricht (NL); Academisch Ziekenhuis Maastricht, Maastricht (NL)

(72) Inventors: Vanessa Lydia Simone LaPointe, Maastricht (NL); Pere Catala Quilis, Maastricht (NL); Mor Mordechai Dickman, Maastricht (NL)

(21) Appl. No.: 19/151,293

(22) PCT Filed: Jan. 29, 2024
(86) PCT No.: PCT/NL2024/050038

§ 371 Date: Jul. 25, 2025

(30) Foreign Application Priority Data
Jan. 27, 2023 (EP) 23153812.5

(51) Int. Cl. A61K 35/30; C12N 5/079; G01N 33/533; G01N 33/68

(52) U.S. Cl. CPC A61K 35/30; C12N 5/0621; G01N 33/533; G01N 33/6893; G01N 2333/70585; G01N 2333/70596

(57) ABSTRACT

The present invention provides a method for determining the quality of cultured corneal endothelial cells (CECs) comprising measuring in or on a cultured CEC a level of expression of the genes Thrombospondin-2 (THBS2) and/or Vitelline Membrane Outer Layer 1 Homolog (VMO1), and determining that the quality of said cultured CEC is non-therapy-grade if said measured level of expression corresponds to a THBS2⁺ and/or VMO1⁺ expression profile.

### CROSS REFERENCE TO RELATED APPLICATIONS

This application is a 371 National Stage application of International Application No. PCT/NL2024/050038, filed Jan. 29, 2024, which claims the benefit of European Patent Application 23153812.5, filed Jan. 27, 2023, each of which is incorporated herein by reference in its entirety.

### FIELD OF THE INVENTION

The present invention involves markers for selecting cultured corneal endothelial cells (CECs) that resemble native CECs to the exclusion of lower grade cells, markers for assessing the quality of cultured CECs, and methods of using the same.

### BACKGROUND OF THE INVENTION

The cornea is the transparent front part of the eye that covers the iris, pupil, and anterior chamber transmitting light into the eye. The inner part of this avascular tissue is covered by a monolayer of hexagonal corneal endothelial cells (CECs) that maintain corneal transparency and hydration by their pump activity and barrier function. Human CECs are arrested in a non-proliferative state and lack regenerative capacity. Consequently, damage to CECs due to surgery or trauma, (inherited) diseases or acquired conditions that affect the corneal endothelium directly, inflammation and other ocular diseases may result in corneal endothelium dysfunction, leading to impaired vision or blindness.

Corneal transplantation using donor tissue is the current therapy for treating corneal endothelium dysfunction. However, only one donor cornea is available for every 70 patients in need, leaving 12.7 million people awaiting treatment worldwide. The cultivation of primary CECs isolated through enzymatic digestion from a corneal endothelium and their expansion in culture has broken the one-donor-one-recipient paradigm, and clinical trials have shown that such primary cultivated CECs can restore corneal transparency. Encouraged by the long-term success of this therapy, clinical trials are ongoing in Japan, Mexico and Singapore to assess the therapeutic potential of cultured CECs.

Nonetheless, the cultivation of CECs has limitations that prevent its wider adoption. At present, primary CEC cultures are only successful when derived from younger donors, e.g. younger than 45 years of age, limiting the pool of donor corneas suitable for this technique. Furthermore, in vitro expansion of primary CECs can presently only yield cells to a few passages with subsequent passaging resulting in dedifferentiation into fibroblast-like cells with loss of CEC phenotypic markers, typically surface markers such as cluster-of-differentiation (CD) antigens. Culture heterogeneity and significant alteration in phenotype and functionality of CECs is often observed after the second passage of cultured primary CECs. The low proliferation and rapid loss of phenotype of cultured primary CECs remains to be solved. Hence, alternative cell sources for CEC primary culture have been investigated, including induced pluripotent stem cells (iPSCs). Such developments hold great promise, but there is still a lack of clear parameters to identify cultured CECs that are of therapy-grade from cells that are of lower grade and unsuitable for transplantation. Surface markers such as CD166, CD44, CD105, CD24, CD26, and CD133, are frequently used as markers for evaluating the quality of cultured CECs, wherein CD166 is used as a positive marker for CECs and remaining markers are negative makers to exclude the fibroblastic-like phenotype (Toda et al., 2017. Investig. Ophthalmol. Vis. Sci. 58, 2011-2020). There is, however, still a need to identify additional or other cell-specific markers that allow to assess the quality of cultured CECs and to select and enrich for native-like CECs that can be used in therapy.

### SUMMARY OF THE INVENTION

The present inventors used single-cell RNA sequencing (scRNA-Seq) to profile 42,220 primary human CECs from six corneas of three donors at five time points over three passages in culture in order to deconstruct the heterogeneity and gain knowledge on the alterations arising from the primary culture of CECs. The inventors found that the culture diversified over time into heterogeneous subpopulations including cells less desirable for therapy that were entering a senescent or fibrotic state. The inventors identified markers that can be used alone or in combination to assess for native-like CECs and enrich for desired cell populations. Pseudo time analysis further uncovered the different trajectories arising during culture. Immunofluorescence analysis confirmed that several markers were exclusively expressed by good quality CECs and not expressed in cultures containing CECs with altered morphology, and that other markers were exclusively expressed by low quality CECs and not expressed in high quality CEC cultures. Together, the inventors' findings shed light on the various routes followed by CECs in culture, and provide novel markers to increase culture efficiency, to improve cultivation protocols, and to select a cell population for clinical applications.

In a first aspect, the present invention provides a method for determining the quality of cultured corneal endothelial cells (CECs) comprising measuring in or on a cultured CEC a level of expression of the genes Vitelline Membrane Outer Layer 1 Homolog (VMO1) and/or Thrombospondin-2 (THBS2), and determining that the quality of said cultured CEC is non-therapy-grade if said measured level of expression corresponds to a VMO1⁺ and/or THBS2⁺ expression profile.

In preferred embodiments of a method of the invention, said level of expression is measured as the absence or presence of the protein expression product of said genes, and wherein it is determined that the quality of said cultured CEC is non-therapy-grade if said protein expression product is detected, preferably wherein absence or presence of the protein expression product is measured by using fluorescently labeled antibodies.

In other preferred embodiments of a method of the invention, said method further comprises measuring in or on said cultured CEC a level of expression of the gene Cingulin-like 1 (CGNL1), and determining that the quality of said cultured CEC is non-therapy-grade if said measured level of expression corresponds to a VMO1⁺, THBS2⁺, and/or CGNL1⁻ expression profile.

In other preferred embodiments of a method of the invention, said method further comprises measuring in or on said cultured CEC a level of expression of at least one, preferably all, of CD166, CD56, CD44 and CD10, and determining that the quality of said cultured CEC is non-therapy-grade if said measured level of expression corresponds to a CGNL1⁻, VMO1⁺, THBS2⁺, CD166⁻, CD56⁻, CD44⁺, and/or CD10⁺ expression profile, or determining that the quality of said cultured CEC is therapy-grade if said measured level of expression corresponds to a CGNL1⁺, VMO1⁻, THBS2⁻, CD166⁺, CD56⁺, CD44⁻, and CD10⁻ expression profile.

In other preferred embodiments of a method of the invention, said method further comprises measuring in or on said cultured CEC a level of expression of at least one gene selected from Amyloid Beta Precursor Protein (APP), S100 Calcium Binding Protein A10 (S100A10), Thioredoxin Reductase 1 (TXNRD1), Tropomyosin 2 (TPM2), Potassium Voltage-Gated Channel Subfamily E Regulatory Subunit 4 (KCNE4), Platelet Derived Growth Factor Receptor Alpha (PDGFRA), and 4-Hydroxyphenylpyruvate Dioxygenase (HPPD), and determining that the quality of said cultured CEC is non-therapy-grade if said measured level of expression corresponds to a APP⁻, S100A10⁺, TXNRD1⁺, TPM2⁺, KCNE4⁺, PDGFRA⁺, and/or HPPD⁺ expression profile, or determining that the quality of said cultured CEC is therapy-grade if said measured level of expression corresponds to a CGNL1⁺, VMO1⁻, THBS2⁻, CD166⁺, CD56⁺, CD44⁻, CD10⁻ APP⁺, S100A10⁻, TXNRD1⁻, TPM2⁻, KCNE4⁻, PDGFRA⁻, and/or HPPD⁻ expression profile.

In yet other preferred embodiments of a method of the invention, said cultured CEC is a cultured primary CEC; a transdifferentiated endothelial precursor cell, including but not limited to a an induced bone marrow-derived endothelial progenitor cell (BEPC); or a stem-cell-derived CEC, including but not limited to an induced pluripotent stem cell (iPSC), an embryonic stem cell (ESC), a neural crest (NC) stem cell, a mesenchymal stem cell (MSC), and a dental pulp stem cell (DPSC), preferably of human origin.

In further preferred embodiments of a method of the invention, said CEC is an individual CEC, more preferably wherein the level of expression is determined in or on a single cell.

In still further preferred embodiments of a method of the invention, said measurement is performed on a cultured CEC at confluency in passages 0, 1, or 2.

In still further preferred embodiments of a method of the invention, said measurement is performed on a cultured CEC cultured in the presence of a ROCK Kinase inhibitor.

In still further preferred embodiments of a method of the invention, the level of expression is measured by immunofluorescence techniques, preferably using fluorescently labeled antibodies. Very suitable antibodies for use in aspects of this invention include polyclonal, preferably monoclonal antibodies, or fragments thereof, preferably against the human antigens, such as anti-CGNL1, anti-VMO1, anti-THBS2, anti-APP, anti-S100A10, anti-TXNRD1, anti-TPM2, anti-KCNE4, anti-PDGFRA, and anti-HPPD. All of these antibodies are readily available from commercial sources.

In further aspects, the present invention provides the use of the expression level of at least one at least one gene selected from CGNL1, VMO1, THBS2, APP, S100A10, TXNRD1, TPM2, KCNE4, PDGFRA, and HPPD as a marker for determining the therapy-grade quality of cultured CECs. Preferably said use comprises measuring the level of expression of at least one of VMO1 and THBS2.

In another aspect, the present invention provides a method of excluding or selecting a culture of CECs for medical use, the method comprising:
a) providing an in vitro culture of CECs, preferably cultured primary CECs or stem-cell-derived CECs, more preferably human CECs,
b) performing the method of any one of claim 1-10 on at least a part of said culture, and
c) excluding said culture of CECs as a culture of CECs unsuitable for medical use when more than 10-15% of the cell population in said culture of CECs is of non-therapy-grade quality or when less than 85-90% of the cell population in said culture of CECs is of therapy-grade quality, or
selecting said culture of CECs as a culture of CECs for medical use when the cell population in said culture of CECs comprises CECs of which less than 10-15% is of non-therapy-grade quality or when more than 85-90% of the cell population in said culture of CECs is of therapy-grade quality.

In another aspect, the present invention provides a method of producing a culture of CECs for medical use, comprising performing the method of claim 11, wherein step c) comprises selecting said culture of CECs as a culture of CECs for medical use, and wherein said method further comprises the step of providing a culture of CECs by:
harvesting the in vitro culture of CECs as culture of CECs for medical use when the incidence of therapy-grade quality CECs in said culture is at a desired level; or
separating therapy-grade quality CECs from said in vitro culture of CECs and collecting the thus separated cells as a culture of CECs for medical use; or
removing non-therapy-grade quality CECs from said in vitro culture of CECs and collecting the remainder of said culture as a culture of CECs for medical use.

In preferred embodiments of methods of selecting or producing a culture of CECs for medical use, step a) comprises cultivating said CECs as a monolayer, optionally dissociating said monolayer, and providing CECs in said optionally dissociated monolayer with fresh tissue culture reagent to produce a subsequent passage, preferably wherein said cultivation occurs in the presence of a ROCK Kinase inhibitor and/or a p53 inhibitor, and/or preferably wherein the CECs are cultivated in or on a substrate or scaffold.

In another aspect, the present invention provides a culture of CECs, preferably in the form of a monolayer, wherein the cell population in said culture of CECs comprises CECs of which at least 85-90% is of therapy-grade quality as defined above, preferably wherein said culture is selected or produced by the methods of selecting or producing a culture of CECs for medical use as defined above, and wherein said culture optionally comprising antibodies against a protein expression product of at least one gene selected from CGNL1, VMO1, THBS2, APP, S100A10, TXNRD1, TPM2, KCNE4, PDGFRA, and HPPD, preferably selected from CGNL1 and APP.

In another aspect, the present invention provides a tissue engineered graft material comprising a culture of CECs according to the invention as described above.

In another aspect, the present invention provides a method for treating a patient suffering from corneal endothelial dysfunction comprising administering to said patient the culture of CECs of the invention as described above, or the tissue engineered graft material of the invention as described above.

In another aspect, the present invention provides a computer-implemented method for determining the quality of cultured CECs comprising steps of:
a) receiving data representing a level of expression of THBS2 and/or VMO1, preferably further including CGNL1, CD166, CD56, CD44 and CD10, said level of expression having been measured in or on at least one cultured CEC, and
b) determining the quality of a cultured CEC based on comparing said data received under a) with reference data of the level of expression in a therapy-grade and/or non-therapy-grade reference CEC, wherein a CD166⁺, CD56⁺, CGNL1⁺, CD44⁻, CD10⁻, VMO1⁻, and THBS2⁻ expression profile is indicative of the therapy-grade quality of said cultured CEC, and wherein a VMO1⁺, THBS2⁺, CGNL1⁻, CD166⁻, CD56⁻, CD44⁺, and/or CD10⁺, expression profile is indicative of the non-therapy-grade quality of said cultured CEC
wherein step b) is preferably performed on a computer using an algorithm wherein the data received in step a) are compared with reference data, preferably wherein said reference values are stored on an accessible memory, and preferably wherein said algorithm can receive the data of step a).

In another aspect, the present invention provides a data processing system comprising a processor adapted to perform the steps of the computer-implemented method for determining the quality of cultured CECs as described above, or a computer program product comprising instructions which, when the program is executed by a computer, cause the computer to carry out the computer-implemented method for determining the quality of cultured CECs, optionally comprising a machine learning data processing model.

### DESCRIPTION OF THE DRAWINGS

FIG. 1. Human CECs were successfully isolated and cultured for scRNAseq. (A) Schematic representation of the experimental overview. (B) Phase contrast images of CEC over the different time points selected for scRNAseq confirm the desired hexagonal morphology of the cells. Scale bars represent 100 μm. (C) Immunofluorescence assessment of CEC markers CD166 and ZO-1 confirm the phenotype of the primary cultured cells at passage 2. Scale bars represent 50 μm.

FIG. 2. scRNAseq analysis reveals distinct clusters of primary cultured CECs (numbered 0-5). (A) UMAP of the 42,220 sequenced cells reveals six cell clusters 0-6. (B) Gene expression UMAP of typical CEC markers namely ALCAM (CD166), PRDX6, PITX2, SLC4A11, and ATP1A1 confirms the endothelial identity of the sequenced cells. (C) Violin plots of cell clusters 0-5 show gene expression for markers of endothelium (ALCAM, COL4A3), senescence (CDKN2A, TAGLN), proliferation (MKT67, CENPF), and fibrosis (CD44, ACTA2). (D) Bar charts show the distribution of cells of each donor per cluster (top panel) and the time point composition (in passage and days) of each cluster (bottom panel).

FIG. 3. scRNAseq analysis reveals seven distinct CEC clusters (C0-C6) at therapeutically relevant time points. (A) UMAP of the 37,158 cells at confluency time points reveals seven cell clusters. (B) Violin plots show gene expression for markers of endothelium (SLC4A11, ALCAM, ATP1A1), proliferation (MKI67), senescence (CDKN2A, TAGLN), fibrosis (CD44, ACTA2), and extracellular matrix production (COL4A2) for the seven clusters. (C) Gene set enrichment analysis (GSEA) reveal differentially expressed gene sets across cell clusters (p<0.05). (D) Significant differentially expressed hallmark gene sets across clusters (p<0.05) show endothelial to mesenchymal transition in lower quality CEC clusters C2, C4 and C5; and proliferation hallmarks in cluster C3. (E) Bar charts show composition of cell clusters across the different sequenced samples (left panel), and across the different time points (right panel). (F) Heatmap of the top 50 differentially expressed genes across clusters C3 and C5 (p<0.01) shows a subpopulation of CEC within cluster C3 that highly resembles the cells comprising cluster C5. Heatmap was performed in R (version 4.2.0). (G) UMAP of the reclustering analysis of cell clusters C3 and C5 with original clusters (left panel) and newly detected clusters (right panel).

FIG. 4. Therapy-grade primary cultured CECs resemble human native CECs. (A) Integrated data UMAP of primary cultured CECs at confluency and native human CECs from the previously published cornea cell atlas (Catalá et al., 2021. Sci. Rep. 11, 21727). The integration analysis shows that native CECs (Atlas 0 and Atlas 1) cluster adjacent to high quality primary cultured CEC clusters (clusters C0, C1, and C6) suggesting clusters C0, C1 and C6 can be considered therapy-grade primary cultured CECs. (B) The cell type of the atlas CECs was predicted using the CEC confluency dataset using the cell label transfer functionality from Seurat. 99.4% of the atlas cells were attributed to clusters C0, C1 and C6, further suggesting the therapeutic standard of these clusters.

FIG. 5. Pseudo temporal trajectory reconstruction reveals the dynamics of primary cultured CECs. (A) Monocle 3 pseudo temporal trajectory reconstruction on UMAP reduction of the scRNAseq confluency time points reveals the CEC cluster dynamics over primary culture. UMAP reduction is colored by pseudo time bins with dark blue being the earliest and yellow corresponding to late. (B) Monocle 3 pseudo temporal trajectory reconstruction on UMAP reduction of the scRNAseq confluency time points excluding proliferative cluster C3 reduces bias and reveals the temporal dynamics of the CEC clusters at confluence level. UMAP reduction is colored by pseudo time bins with dark blue being the earliest and yellow corresponding to late. (C) Pseudo time reconstruction reveals differential gene expression trends over CEC clusters. Our analysis revealed a reduction over time of CEC markers SLC4A11, ATP1A1, COL4A3, and ALCAM, while PRDX6 expression was constant. Conversely, CD44, MME, and VMO1 expression significantly increased over time.

FIG. 6. scRNAseq analysis suggests specific markers for CEC quality assessment. (A) Gene expression UMAP of differentially expressed markers in clusters of therapy-grade CECs (clusters C0 and C1) and clusters of senescent/fibrotic CECs (cluster C5). NCAM1, CGNL1, and ALCAM were differentially expressed in clusters C0 and C1 (p<0.01). MME and CD44 were differentially expressed in clusters C2 and C5. THBS2 and VMO1 were differentially expressed in cluster C5 (p<0.01). (B) Phase contrast images of a high quality therapy-grade CEC culture showing the typical hexagonal cell morphology and a low quality culture of primary human CECs showing the characteristic morphological alterations of an endothelial to mesenchymal transition. Scale bars represent 100 μm. Immunofluorescence analysis shows expression of CD166 (green, bright in B/W), NCAM1 (red, bright in B/W), and CGNL1 (red, bright in B/W) in high quality CEC cultures (N=2) and absence of expression (not visible in B/W) in lower quality CEC cultures (N=2) (top two rows of 12 photographs in total). Immunofluorescence analysis shows expression of CD44 (green, bright in B/W), MME (CD10) (red, bright in B/W), THBS2 (red, bright in B/W), and VMO1 (red, bright in B/W) in lower quality, non-therapy-grade, CEC cultures (N=2) and the absence of expression (not visible in B/W) in high quality CEC cultures (N=2) (bottom three rows of 18 photographs in total). Cell nuclei were stained with Hoechst 33342 (blue, bright in B/W, visible in 9 photographs of high quality primary hCECs). Scale represent 100 μm.

FIG. 7. Phase contrast images of each donor time point used for scRNAseq, related to FIG. 1. Phase contrast imaging shows desired endothelial morphology in all of the sequenced samples. Scale bar represents 100 μm.

FIG. 8. UMAP projection of cells per time point, related to FIG. 2. Cell distribution UMAP per each time point further confirms that cluster is composed of CEC at early culture time points. Each donor is represented by color confirming the donor distribution across cell clusters. P0D2: day 2 in proliferation media. P0D5: day 5 in proliferation media. P0D14: passage 0 maintenance media. P1D30: passage 1 maintenance media. P2D46: passage 2 maintenance media.

FIG. 9. scRNAseq revealed absence of stroma and epithelial contamination, related to FIG. 2. Gene expression UMAP of stromal markers KERA, LUM, and ALDH1A1 and epithelial markers KRT12, KRT14, and PAX6 confirmed absence of contaminant corneal side-populations in the primary culture.

FIG. 10. Violin plots of differentially expressed genes for cluster identification, related to FIG. 2. Violin plots of differentially expressed genes for cluster annotation. Corneal endothelium: ATP1A1, COL4A3, CDH2, ALCAM, SLC4A11, PITX2. Senescence: MT2A, CDKN2A, TAGLN. Proliferation: MKI67, CENPF, PTTG1. Fibrosis and endothelial to mesenchymal transition: ACTA2, CD44, COL6A1.

FIG. 11. Cluster 3 is enriched in ribosomal gene expression. (A) UMAP representation of ribosomal gene fraction shows that cluster 3 expresses a high amount of ribosomal genes compared to other clusters. (B) Violin plot of ribosomal gene fraction shows further confirms a high expression of ribosomal genes in cluster 3 compared to the other detected clusters.

FIG. 12. UMAP projection of cells per time point at confluency, related to FIG. 3. Cell distribution UMAP per each time point at confluency level shows homogeneous distribution of donors across all cell clusters. P0D14: passage 0 maintenance media. P1D30: passage 1 maintenance media. P2D46: passage 2 maintenance media.

FIG. 13. Violin plots of differentially expressed genes for cluster identification, related to FIG. 3. Violin plots of differentially expressed genes for cluster annotation at the confluency time points. Corneal endothelium: SLC4A11, ATP1A1, ALCAM, CDH2. Corneal endothelium extracellular matrix: COL4A6, COL4A3, COL4A1, COL4A2, COL5A2. Senescence: CDKN2A, TAGLN, MT2A, CDKN1A, LGALS1. Cell secretion: GOLGA8A. Proliferation: MKI67, CENPF, PTTG1. Fibrosis and endothelial to mesenchymal transition: COL6A3, CD44, FBLN5, COL6A1, ACTA2.

FIG. 14. Gene expression UMAP of ALCAM (CD166), CD44 and ACTA2, related to FIG. 3. Gene expression UMAP shows heterogeneous expression of ALCAM, CD44 and ACTA2 across cell cluster C3.

FIG. 15. Identification of putative doublets. Putative doublets were identified with scDblFinder. The identified putative doublets were dispersed across all cell clusters and did not bias scRNAseq clustering therefore were not removed.

### DETAILED DESCRIPTION OF THE INVENTION

### Definitions

The term “primary CEC”, as used herein, refers to a CEC that is isolated from the corneal endothelium (cornea) of an individual, preferably a human individual. Primary CECs are by definition of therapy-grade, as such cells are used in transplantation.

The terms “cultured corneal endothelial cell” and “cultured CEC”, as used herein, refers to a CEC or CEC-like cell that is or has been cultivated in a growth medium. CECs may be cultivated in order to expand the number of cells obtained from a donor cornea. A “CEC-like cell” is herein referred to as a “native-like CEC”, whose origin may include a primary CEC, a stem-cell derived CEC or a transdifferentiated cell.

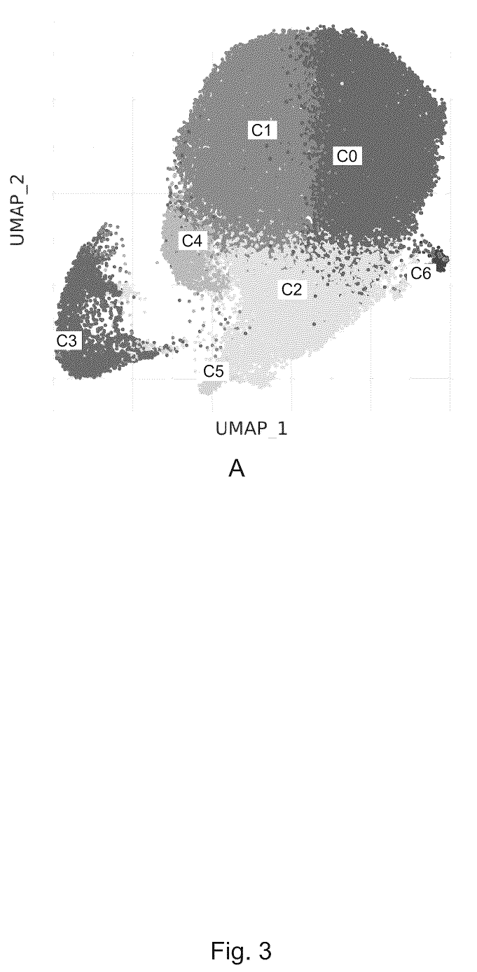

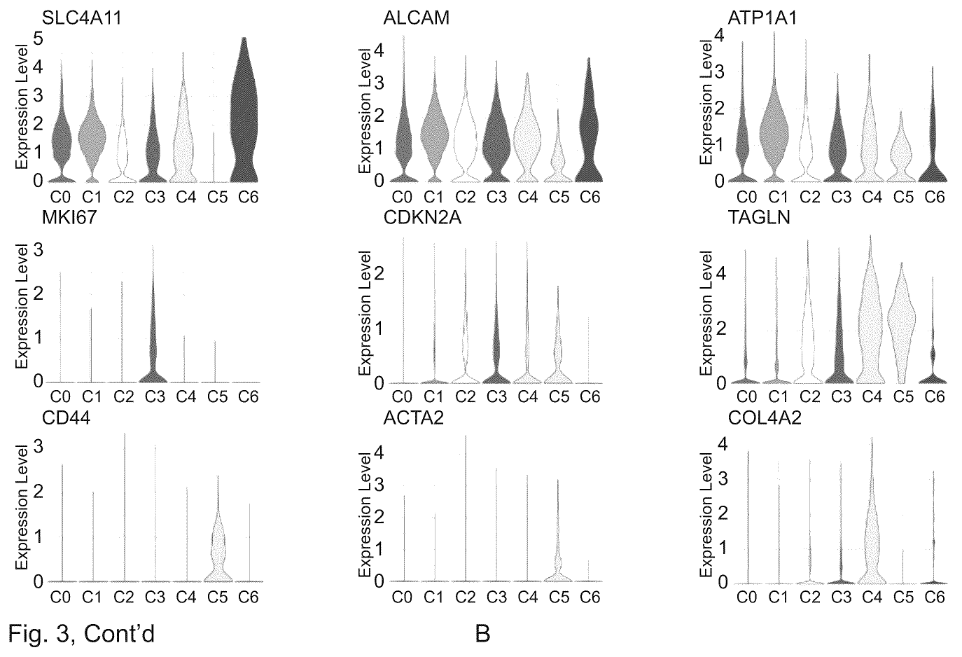

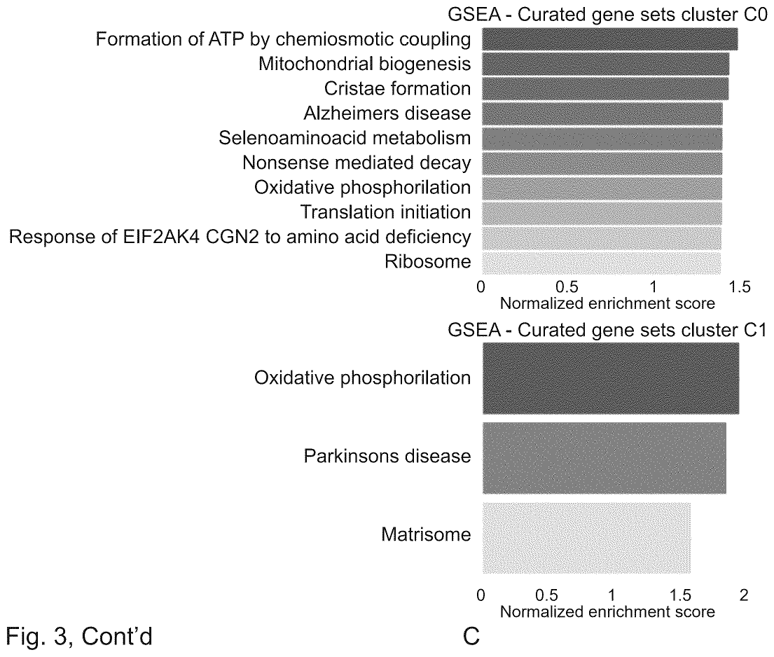

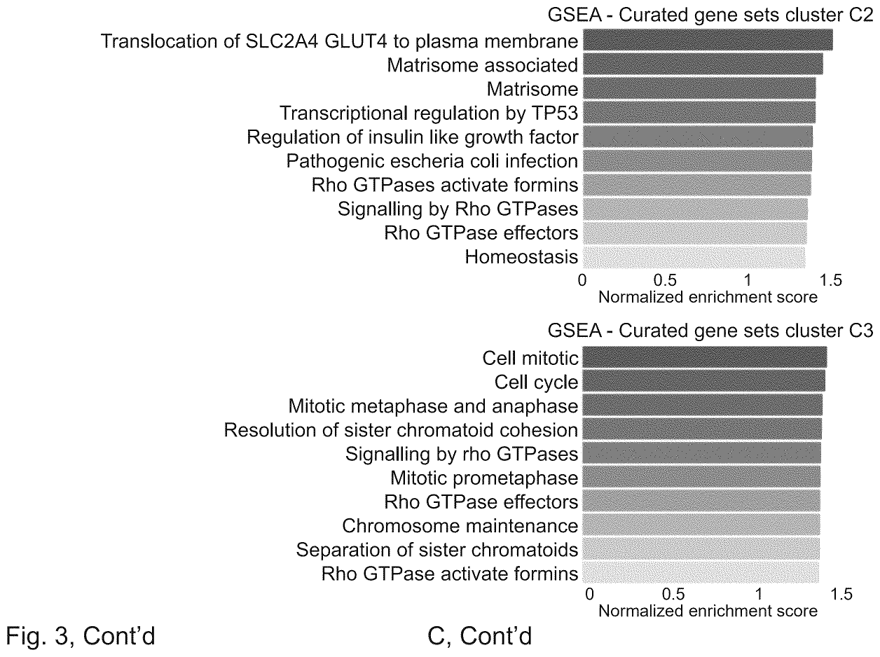

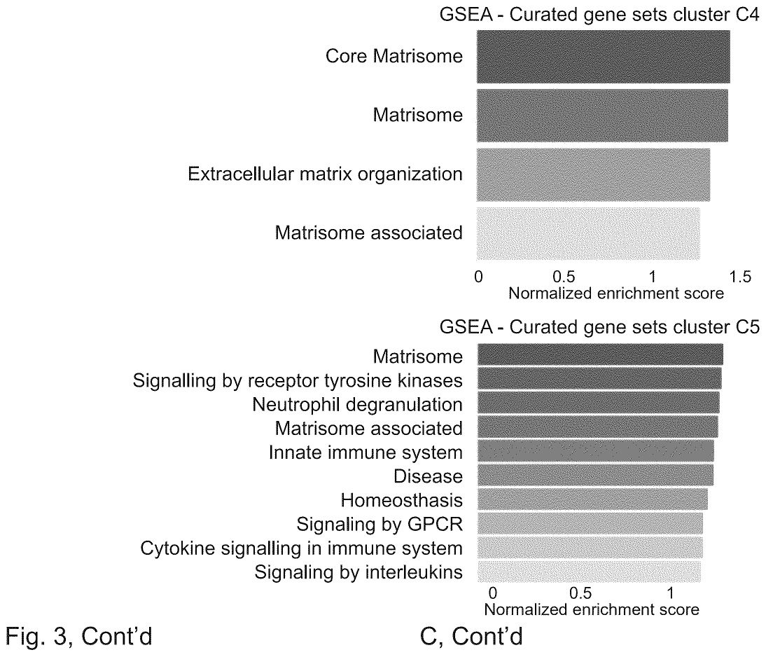

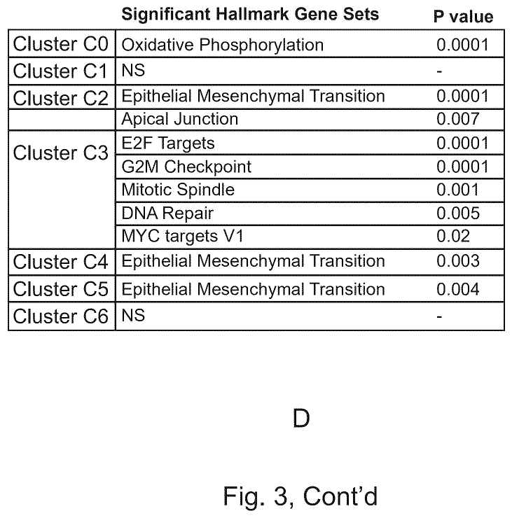

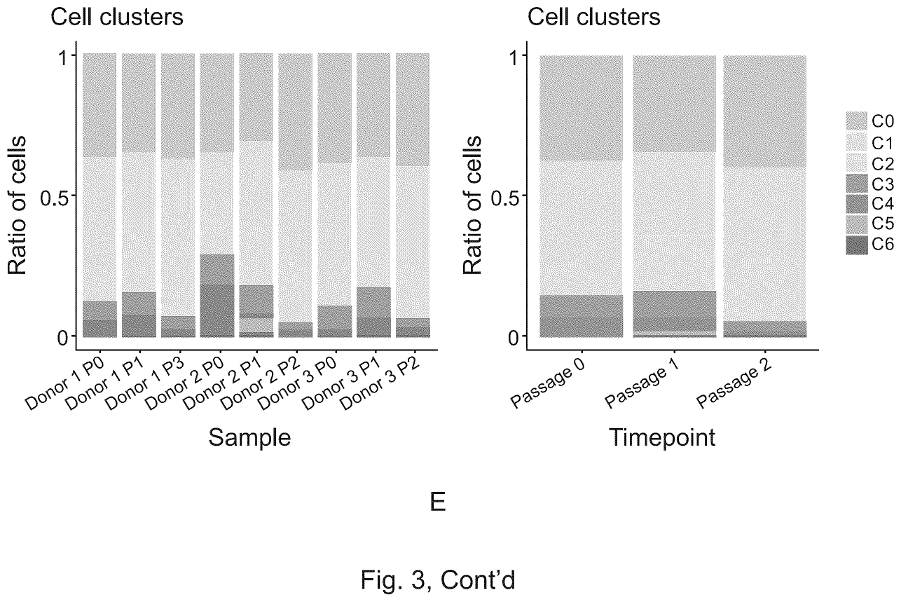

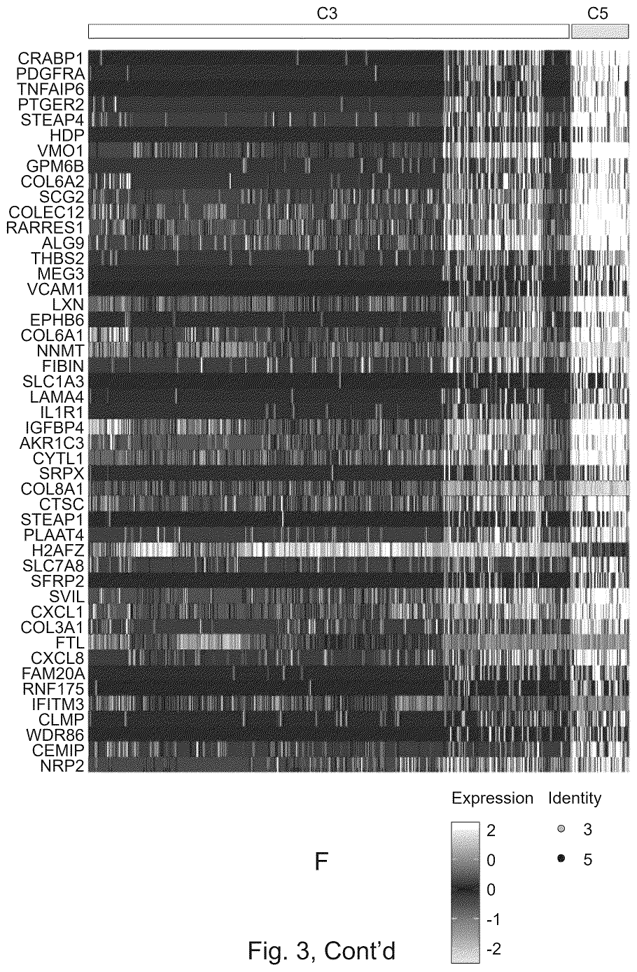

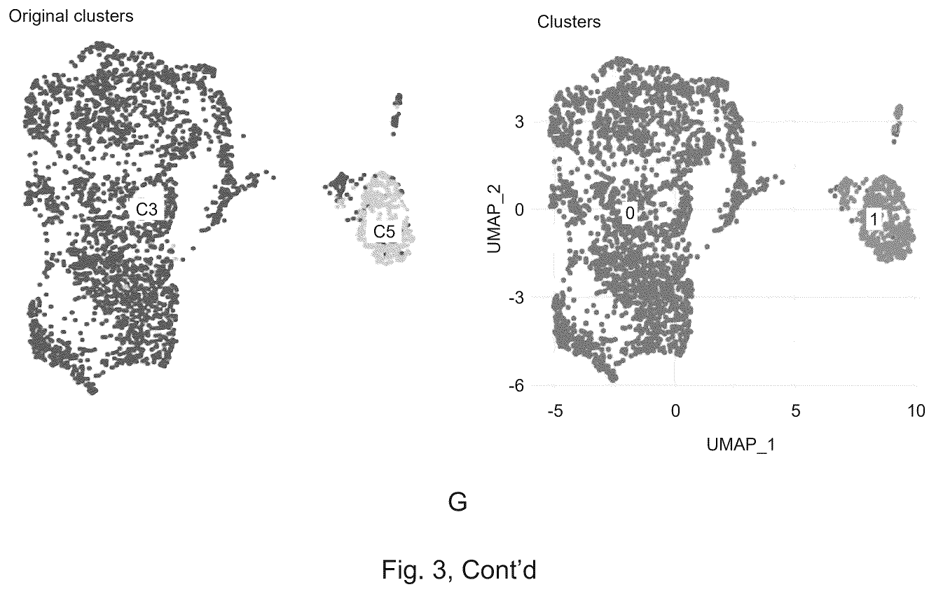

The term “native-like CEC”, as used herein, refers to a cultured CEC that resembles a primary CEC, preferably a CEC in the cornea. The native-like CEC preferably has the functionality that is comparable to a native CEC, such as an active metabolic pump activity, an active barrier function, the surface marker expression profile, the gene expression profile, and/or a protein expression profile of a native CEC. The resemblance to a primary CEC may be determined by analyzing the morphology or shape of a cell, and/or by determining an expression level of one or more genes in said cell. For example, a native-like CEC has a typical polygonal shape and is mononucleated, while a non-native-like CEC may have an elongated or even spindle-like shape and may be polynucleated. An expression level may be determined for one or more of CGNL1, VMO1, THBS2, APP, S100A10, TXNRD1, TPM2, KCNE4, PDGFRA, and HPPD, whereby a native-like CEC represents a CGNL1⁺, VMO1⁻, THBS2⁻, APP⁺, S100A10⁻, TXNRD1⁻, TPM2⁻, KCNE4⁻, PDGFRA⁻, and/or HPPD⁻ phenotype, when compared to a reference level of expression, wherein the reference may be native-like or non-native like, i.e. therapy-grade or non-therapy grade. When referring to a population of cells, the population may be termed a “native-like CEC” population when at least 50%, 60, %, 70% 80%, preferably at least 85-90%, more preferably at least 90-95% or even at least 99%, of the cells are positive for at least one, preferably at least 2 of markers CGNL1 and APP, and at most 20%, preferably at most 10-15%, more preferably at most 1-10% of the cells are positive for at least one, preferably at least two of VMO1, THBS2, S100A10, TXNRD1, TPM2, KCNE4, PDGFRA, and HPPD. Clusters C0, C1 and/or C6 as shown in FIG. 3A to the Example described herein below are representative high quality, therapy-grade, native-like CECs. Native-like CECs may further be characterized by a CD166⁺, CD56⁺, CD44⁻ and CD10⁻ phenotype. One of skill will appreciate that use of one or more of the prior art markers frequently used for evaluating the quality of cultured CECs, such as CD166, CD56, CD44, CD10, CD105, CD24, CD26, and/or CD133, is expressly contemplated herein, wherein a native-like CEC may further be characterized by a CD166⁺, CD56⁺, CD44⁻, CD10⁻, CD105⁻, CD24⁻, CD26⁻, and CD133⁻ phenotype.

The term “represents”, as is used herein when referring to a native-like CEC, refers to a level of expression of at least one of CGNL1 and APP that is increased when compared to a reference level of expression, and/or at least one of VMO1, THBS2, S100A10, TXNRD1, TPM2, KCNE4, PDGFRA, and HPPD that is decreased when compared to a reference level of expression. For example, a CEC in which the level of expression of CGNL1 is increased when compared to a reference level of expression, represents a native-like CEC. The term “represents” preferably refers to a CEC in which two or more markers genes, such as three, four, five, six, seven, eight, nine or all ten marker genes selected from CGNL1, APP, VMO1, THBS2, S100A10, TXNRD1, TPM2, KCNE4, PDGFRA, and HPPD have a level of expression that is increased (CGNL1 and APP), or decreased (VMO1, THBS2, S100A10, TXNRD1, TPM2, KCNE4, PDGFRA, and HPPD, when compared to a reference level of expression.

The term “non-native-like CEC”, as is used herein, refers to a cultured CEC that does not resemble a primary CEC. Said cell may be elongated or have a spindle-like shape and may be polynucleated, and/or resemble a CGNL1−, VMO1+, THBS2+, APP−, S100A10+, TXNRD1+, TPM2+, KCNE4+, PDGFRA+, and/or HPPD+ phenotype, when compared to a reference level of expression. A non-native-like CEC is considered herein to be of insufficient quality to provide for a successful treatment for a corneal endothelial dysfunction. In other words, a non-native-like CEC is generally of non-therapy grade, and may be less suited for cell transplantation. When referring to a population of cells, the population may be termed a “non-native-like CEC” population when less than 85-90%, preferably less than 80%, more preferably less than 70%, 60%, 50% of the cells show increased differential expression of at least one, preferably at least 2 of markers CGNL1 and APP, and at least 10-15%, preferably at least 20%, 30%, 40%, or 50% of the cells show increased differential expression of at least one, preferably at least two of VMO1, THBS2, S100A10, TXNRD1, TPM2, KCNE4, PDGFRA, and HPPD. One of skill will appreciate that use of one or more of the prior art markers frequently used for evaluating the quality of cultured CECs, such as CD166, CD56, CD44, CD10, CD105, CD24, CD26, and/or CD133, is expressly contemplated herein, wherein a non-native-like CEC may further be characterized by a CD166⁻, CD56⁻, CD44⁺, CD10⁺, CD105⁺, CD24⁺, CD26⁺, and CD133⁺ phenotype or expression profile. The term non-native-like is equivalent to the term “non-therapy-grade” as used herein. Methods of the present invention are highly suitable for determining if a cultured CEC is of non-therapy-grade, for instance by determining that the cell expresses such markers as VMO1 and THBS2. Although expression of markers VMO1 and THBS2 may in some instances still be observed at very low levels at the mRNA level in therapy-grade cells, these cells are characterized by the absence of VMO1 and THBS2 protein. Hence, these negative markers are highly suitable as exclusion criteria for producing therapy-grade CECs by cultural proliferation, as such cultivation results (over time) in an increase in the number of CECs in such cultures that are non-therapy grade. Hence, CECs may be harvested from culture while they are (still) of therapy-grade, or cultivation conditions may be developed, adapted or modified that result in lower percentages of non-therapy-grade CECs in such cultures. Also, cultures that contain unacceptable levels of non-therapy-grade CECs (such as more than 10-15% of the population) may be discarded.

As used herein, the term “measuring a level of expression of at least one gene”, refers to measuring gene expression, such as measuring the presence or quantity of a gene expression product, including an mRNA or protein product, expressed from said gene. The term “in or on” with reference to measuring a level of expression of at least one gene, refers to quantitative measurement of the presence of an expression product, such as a nucleic acid expression product or protein expression product, in a cell, or to quantitative measurement of an expression product, such as a protein expression product inter alia in the form of a transmembrane protein or a cell surface marker, on the surface of a cell, respectively. Quantitative measurement may or may not require destruction of the cell.

As used herein the terms “marker” and “biomarker” may be used interchangeably herein and refer to a measurable indicator of some biological state or condition. The marker may be a cell surface marker, a gene expression marker or a protein expression marker, and multiple markers may constitute a surface marker expression profile, a gene expression profile, and/or a protein expression profile. In preferred embodiments, the marker is a protein marker, preferably a cell surface protein.

Expression levels or profiles as defined herein may refer to surface marker expression levels or profiles, gene expression levels or profiles, and/or protein expression levels, that is, to the level or profile of any of the transcriptomic or proteomic expression markers as defined herein, and may encompass any gene or genes, protein or proteins as defined herein whose expression profile or whose occurrence is associated with a specific cell type, subtype, or cell state of a specific cell type or subtype within a population of cells (e.g., native-like versus non-native like CECs). It is to be understood that also when referring to proteins (e.g. differentially expressed proteins), such may fall within the definition of “gene” profile. Levels of expression or activity may be compared between different cells in order to characterize or identify for instance profiles specific for cell (sub)populations. Increased or decreased expression or activity or prevalence of specific genes may be compared between different cells in order to characterize or identify for instance specific cell (sub)populations. The detection of a specific profile in single cells may be used to identify and quantitate for instance specific cell (sub)populations. A profile may include a gene or genes, protein or proteins, whose (level of) expression or occurrence is specific to a cell (sub)population, such that expression or occurrence is exclusive to the cell (sub)population. A gene expression profile as used herein, may thus refer to any set of up- and/or down-regulated genes that are representative of a cell type or subtype. A gene expression profile as used herein, may also refer to any set of up- and/or down-regulated genes between different cells or cell (sub)populations derived from gene-expression analysis of those cells or cell (sub)populations. For example, a gene expression profile may comprise a list of genes differentially expressed between a therapy-grade CEC and a non-therapy-grade CEC. The gene expression profile as defined herein can be used to indicate the presence of a cell type, a subtype of the cell type, the state of the population of cells, a particular cell type population or subpopulation, and/or the overall status of the entire cell (sub)population. The expression profile according to certain embodiments of the present invention may comprise or consist of one or more genes or proteins, such as for instance 1, 2, 3, 4, 5, 6, 7, 8, 9, 10 or more. In certain embodiments, an expression profile is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, an expression profile consists of one or more differentially expressed genes/proteins when comparing different cells or cell (sub)populations, including comparing different cell (sub)populations of CECs within in a culture of CECs, as well as comparing cultured CEC (sub)populations with other cultured CEC (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. Up-regulation of expression of a genetic marker in a cell of interest (e.g., in a high quality or therapy-grade cultured CEC) relative to its expression in a cell that is of less interest (e.g., in a low quality or non-therapy-grade cultured CEC) is also referred to herein as increased differential expression of said genetic marker, whereas down-regulation of expression of a genetic marker in a cell of interest (e.g., in a high quality or therapy-grade cultured CEC) relative to its expression in a cell that is of less interest (e.g., in a low quality or non-therapy-grade cultured CEC) is also referred to herein as decreased differential expression of said genetic marker between the two cell types or (sub)populations. As discussed herein, differentially expressed genes/proteins may be differentially expressed on a single cell level, or may be differentially expressed on a cell population level. Preferably, the differentially expressed genes/proteins as discussed herein, such as constituting the expression profile as discussed herein, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type (e.g., good quality or therapy-grade cultured CECs) which can be distinguished or are uniquely identifiable and set apart from other CECs (e.g., low quality or non-therapy-grade cultured CECs). The cell subpopulation may be phenotypically characterized, and is preferably characterized by the expression profile as discussed herein.

The term “differentially expressed” or “differential expression”, as used herein, refers to a difference in the level of expression of the transcriptomic or proteomic expression markers as defined herein, when comparing different cells or cell (sub)populations, including comparing different cell (sub)populations of CECs within in a culture of CECs, as well as comparing cultured CEC (sub)populations with other cultured CEC (sub)populations, which can be assayed by measuring the level of expression of the products of the genes, such as the difference in level of messenger RNA transcript or of proteins expressed thereof. In a preferred embodiment, the difference is statistically significant. The term “difference in the level of expression” refers to an increase or decrease in the measurable expression level of the transcriptomic or proteomic expression markers as defined herein, for example as measured by the amount of messenger RNA transcript and/or the amount of protein in a sample as compared with the measurable expression level of a given expression marker in a control or reference, such as a reference cell, reference (sub)population or reference sample. In one embodiment, the differential expression can be compared using the ratio of the level of expression of a given marker or markers as compared with the expression level of the given marker or markers of a control or reference, wherein the ratio is not equal to 1.0. For example, an RNA or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less.

The term “corresponds to”, with reference to an expression level or profile, as used herein, indicates that there is a similarity (or high correlation) in the expression level or profile between the CEC of which the quality is to be determined and a reference CEC, meaning that there is no or little difference in the level of expression of the marker(s) between the test sample or profile and the control or reference sample or profile. For example, similarity can refer to a fold difference compared to a control. In a preferred embodiment, the term refers to a situation wherein there is no statistically significant difference in the level of expression of the markers.

As used herein, the term “selecting” refers to a method of distinguishing between cultured CECs of good quality (i.e. native-like CECs) and cultured CECs of low quality (non-native-like CECs). Selection in aspects of the present invention preferably occurs on the basis of transcriptomic or proteomic expression markers as defined herein, wherein good quality CECs exhibit expression of at least one of CGLN1, APP, CD56 (NCAM1) and CD166 (ALCAM), or any other good quality marker as defined herein, while such cells preferably exhibit characteristic hexagonal morphology, and wherein low quality CECs exhibit expression of at least one of CD44, CD10 (MME), THBS2, and VMO1, or any other low quality marker as defined herein, while such cells preferably exhibit spindle shape morphology, a characteristic of cells undergoing an endothelial to mesenchymal transition. A method of selecting in aspects of this invention may comprise the typing of CECs, e.g. based on gene expression levels or profiles as defined herein. A method of selecting preferably allows the subsequent separation of cells that differ in their characteristics. A method of selecting may include or may be followed by a step of sorting, such as by physical separation of a selected cell from non-selected cells. The selecting and sorting process may be destructive or non-destructive to the cultured CEC. If a method of selecting according to the present leads to the death of the CEC, the selecting and sorting may comprise the enrichment for native-like CECs in a culture of CECs by the purging of non-native-like CECs.

The term “reference level of expression”, as is used herein, refers to the average level of expression of the same set of genes in a reference. Said reference may comprise CECs, such as primary CECs, from a single individual, or from at least 2, 5, 10, 50, or 100 individuals. Said reference may also comprise a mixture of native-like and non-native-like cultured CECs obtained from one or more individuals, or obtained from stem cells. In addition, a reference may refer to separate average levels of expression in native-like CECs versus non-native-like CECs.

The difference or similarity between a level of expression of one or more of CGNL1, APP, VMO1, THBS2, S100A10, TXNRD1, TPM2, KCNE4, PDGFRA, and HPPD in cultured CECs and a reference level of expression can be determined by determining a correlation to the level of expression of one or more of CGNL1, APP, VMO1, THBS2, S100A10, TXNRD1, TPM2, KCNE4, PDGFRA, and HPPD in the reference. For example, one can determine whether the level of expression of, for example, CGNL1 and VMO1 correlate to the level expression levels of CGNL1 and VMO1 in a reference. This correlation can be numerically expressed using a correlation coefficient. Several correlation coefficients can be used. Preferred methods are parametric methods which assume a normal distribution of the data. One of these methods is the Pearson product-moment correlation coefficient, which is obtained by dividing the covariance of the two variables by the product of their standard deviations.

Said correlation between the level of expression of one or more of CGNL1, APP, VMO1, THBS2, S100A10, TXNRD1, TPM2, KCNE4, PDGFRA, and HPPD in the cultured CEC or CECs and the reference, can be used to produce an overall similarity score. A similarity score is a measure of the average correlation of the level of gene expression in a cultured CEC or population of CECs, and a reference. Said similarity score can, but does not need to be, a numerical value between +1, indicative of a high correlation between the level of gene expression in the CEC or population of CECs and said reference, and −1, which is indicative of an inverse correlation. A threshold can be used to differentiate between a native-like CEC and a non-native-like CEC. Said threshold is an arbitrary value that allows to differentiate between a native-like CEC and a non-native-like CEC.

The term “donor”, as is used herein, refers to an individual from whom the CEC or stem cell was obtained. A CEC donor preferably has had no history of ocular disease.

The indication “+” following an abbreviation of a gene or protein, such as in “CGNL1+”, as is used herein, refers to expression, or an upregulation in the level of expression, of said gene or protein, i.e., the marker cingulin-like 1 (CGNL1), which expression or upregulation is present in therapy-grade CECs, when compared to non-therapy-grade CECs. Upregulation may also be indicated by increased differential expression herein. As a reference level of expression, the level of expression in a native-like CEC or a primary CEC may be used, wherein the level of expression in those references is indicated herein as “CGNL1+”, meaning that there is a significantly higher expression of that marker in a cell of good quality than in a cell of low quality.

The indication “−” following an abbreviation of a gene or protein, such as in “VMO1−”, as is used herein, refers to an absence or lack of expression, or a downregulation in the level of expression, of said gene or protein, i.e., the marker Vitelline Membrane Outer Layer 1 Homolog (VMO1), which lack of expression or downregulation is present in therapy-grade CECs, when compared to non-therapy-grade CECs. Downregulation may also be indicated by decreased differential expression herein. As a reference level of expression, the level of expression in a native-like CEC or a primary CEC may again be used, wherein the level of expression in those references is indicated herein as “VMO1−” or “THBS2−”, meaning that there is a significantly lower expression of that marker in a cell of good quality than in a cell of low quality.

In some embodiments of aspects of this invention, the reference level of expression of the one or more genes is determined from a population of primary CECs. In some embodiments, the reference level is a median of a Z-score of the normalized expression level of the one or more genes in a population of primary CECs.

In some embodiments of aspects of this invention, the expression level is a nucleic acid expression level. In some embodiments, the nucleic acid expression level is an mRNA expression level. In some embodiments, the mRNA expression level is determined by RNA-seq, RT-qPCR, qPCR, multiplex qPCR or RT-qPCR, microarray analysis, SAGE, MassARRAY technique, ISH, or a combination thereof.

In other embodiments of aspects of this invention, the expression level is a protein expression level. In some embodiments, the protein expression level is determined by immunohistochemistry (IHC), Western blot, enzyme-linked immunosorbent assay (ELISA), immunoprecipitation, immunofluorescence, radioimmunoassay, or mass spectrometry. Preferably, the expression level is determined by immunofluorescence.

The term “immunofluorescence”, as is used herein, refers to a type of immunohistochemistry technique that utilizes fluorophores to visualize various cellular antigens such as proteins. This technique can be utilized to visualize the localization of a protein within a cell, and allows to provide an estimation of the level of expression of said protein by quantifying the fluorescent signal.

The term “monolayer”, as is used herein, refers to a single or essentially single layer of cells. Single cells may during expansion be or become attached to specially treated plastic or glass substrates in tissue culture dishes, such as fibronectin and/or collagen coated substrates, and produce a monolayer of cells. CECs in such a cultivated monolayer may be dissociated by breakage of both intercellular and cell-to-substrate connections by the use of a proteolytic enzyme such as trypsin, collagenase, and/or TrypLE select or TrypLE express (both of Thermo Fisher Scientific; Waltham, MA, USA). After the cells have been dissociated into a suspension comprising single cells, they may be transferred onto culture plates comprising fresh culture medium to produce a subsequent passage of cells, or they may be used in methods according to the invention. Monolayers of CECs or cell suspensions of CECs may also be combined with a tissue engineering support, such as a scaffold comprising a synthetic, biological, (bio)mimetic or hybrid scaffold material as described herein, for instance by seeding the cells onto the tissue engineering support, so as to become attached for instance in the form of a monolayer.

The term “passage of cells”, as is used herein, refers to the splitting or subculturing of cells in order to provide adequate space, nutrients, and environmental conditions to keep cells proliferating. Passaging of adherent cells such as CECs refers to the detachment of cells from a tissue culture dish, and the transfer of an appropriate dilution to a new tissue culture dish comprising fresh growth media. A passage number refers to the number of times cells have been passaged.

The term “transplantation”, as is used herein, refers to a method of transplanting cells or grafts in a subject, in particular comprising implanting in the subject cultured CECs prepared or obtained in accordance with the present invention. Cultured CECs for transplantation in aspects of this invention are typically allogeneic to the subject which will receive the transplantation, although autologous transplantations are also within the ambit of the present invention.

The term “for medical use”, as used herein, includes reference to any form of clinical use of the CECs of the present invention, be it in methods for treatment of the human or animal body by surgery or therapy or diagnostic methods practiced on the human or animal body, or as a product for use in any of these methods. Examples of medical use include, but are not limited to, use as a medicament, use in therapy, use in regenerative medicine, use in treatment of corneal endothelium dysfunction. Such use may generally include administration of the CECs of the present invention to patients in need thereof. Further examples of medical use may, for instance, include the use of the CECs of the present invention in drug development, drug screening, and disease modelling.

The terms “for use in therapy”, “for use in regenerative medicine” and “for treating corneal endothelium dysfunction”, with reference to the use of the CECs of the present invention, can be used interchangeably herein, and will typically involve administration of the cells into a patient or subject in need thereof, such as by transplantation, and is therefore an indication that the CECs or grafts comprising said CECs are of a quality that is sufficient or suitable for such purposes. CECs for medical use are preferably “native-like CECs”, as defined herein.

The term “drug development”, as used herein, refers to the process of preclinically testing the efficacy, tolerance and safety of drug candidates in cell cultures.

The term “drug screening”, as used herein, refers to the use of cells and tissues in the laboratory to identify drugs with a specific function.

The term “disease modelling”, as used herein, refers the use of cell cultures or tissues as a disease model—a biological system in the lab that mirrors a disease or shows some of the same disease processes to evaluate the effect of health intervention strategies.

The term “tissue culture dish”, as is used herein, refers to a receptacle such as a flask, tube, or plate that allows the culturing of cells or tissue in a specialized medium. Sterile conditions are maintained to prevent contamination with microorganisms. A tissue culture dish may be a glass dish, or a plastic dish such as a polystyrene, polyethylene, polyvinyl chloride and polypropylene (PP). Cells may be grown as suspension cells or as adherent cells. The surface of a tissue culture dish onto which adherent cells are grown may have been treated, for example by plasma treatment, as is known to a person skilled in the art. Said surface may be coated with a substrate such as poly-l-lysine, poly-l-ornithine, collagen type IV, fibronectin, and laminin. A tissue culture dish may be identified by its surface area, e.g., 25 cm², 75 cm² flasks, its diameter, or the number of wells, such as a six well plate, 24 well plate, 96 well plate or 384 well plate.

The term “tissue engineered graft material”, as used herein, refers to an tissue graft as a combination of cells and a scaffold material, optionally further comprising biologically active molecules, engineered into a functional tissue. The goal of tissue engineering is to assemble functional constructs that restore, maintain, or improve damaged tissues or whole organs.

The term “Amyloid Beta Precursor Protein (APP)”, as is used herein, refers to a cell surface receptor and transmembrane precursor protein. The gene encoding APP is specified by HGNC accession code 620 and Ensembl accession code ENSG00000142192. Known RefSeq cDNA sequences are provided by GenBank accession numbers NM_000484.4, NM_001136016.3, NM_001136129.3, NM_001136130.3, NM_001136131.3 NM_001204301.2, NM_001204302.2, NM_001204303.2, NM_001385253.1, NM_201413.3, and NM_201414.3. The protein is specified by UniProt accession code P05067.

The term “Cingulin-like 1 (CGNL1)”, as is used herein, refers to a protein that localizes to the apical junction complex of both adherens and tight cell-cell junctions and mediates junction assembly and maintenance by regulating the activity of the small GTPases RhoA and Rac1. The gene encoding CGNL1 is specified by HUGO Gene Nomenclature Committee (HGNC) accession code 25931 and Ensembl accession code ENSG00000128849. Known Reference Sequences (RefSeq) cDNA sequences are provided by GenBank accession numbers NM_001252335.2 and NM_032866.5. The protein is specified by UniProt accession code Q0VF96.

The term “4-Hydroxyphenylpyruvate Dioxygenase (HPPD)”, as is used herein, refers to an enzyme that catalyzes the conversion of 4-hydroxyphenylpyruvate to homogentisate. The gene encoding HPPD is specified by HGNC accession code 5147 and Ensembl accession code ENSG00000158104. Known RefSeq cDNA sequences are provided by GenBank accession numbers NM_001171993.2 and NM_002150.3. The protein is specified by UniProt accession code P32754.

The term “Potassium Voltage-Gated Channel Subfamily E Regulatory Subunit 4 (KCNE4)”, as is used herein, refers to a type I membrane protein that is a member of the potassium channel, voltage-gated, I_{sK}-related subfamily. The gene encoding KCNE4 is specified by HGNC accession code 6244 and Ensembl accession code ENSG00000152049. A known RefSeq cDNA sequence is provided by GenBank accession number NM_080671.4. The protein is specified by UniProt accession code Q8WWG9.

The term “Platelet Derived Growth Factor Receptor Alpha (PDGFRA)”, as is used herein, refers to a cell surface receptor tyrosine kinase receptor for members of the platelet-derived growth factor family. The gene encoding PDGFRA is specified by HGNC accession code 8803 and Ensembl accession code ENSG00000134853. Known RefSeq cDNA sequences are provided by GenBank accession numbers NM_001347827.2, NM_001347828.2, NM_001347829.2, NM_001347830.2, and NM_006206.6. The protein is specified by UniProt accession code P16234.

The term “S100 Calcium Binding Protein A10 (S100A10)”, as is used herein, refers to a member of the S100 family of proteins that is localized in the cytoplasm and/or nucleus of cells. The gene encoding S100A10 is specified by HGNC accession code 10487 and Ensembl accession code ENSG00000197747. A known RefSeq cDNA sequences is provided by GenBank accession number NM_002966.3. The protein is specified by UniProt accession code P60903.

The term “Thrombospondin-2 (THBS2)”, as is used herein, refers to a glycoprotein that mediates cell-cell and cell-matrix interactions. The gene encoding THBS2 is specified by HGNC accession code 11786 and Ensembl accession code ENSG00000186340. Known RefSeq cDNA sequences are provided by GenBank accession numbers NM_001381939.1, NM_001381940.1, NM_001381941.1, NM_001381942.1, and NM_003247.5. The protein is specified by UniProt accession code P35442.

The term “Tropomyosin 2 (TPM2)”, as is used herein, refers to a member of the actin filament binding protein family that is mainly expressed in slow, type 1 muscle fibers. The gene encoding TPM2 is specified by HGNC accession code 12011 and Ensembl accession code ENSG00000198467. Known RefSeq cDNA sequences are provided by GenBank accession numbers NM_001301226.2, NM_001301227.2, NM_003289.4, NM_213674.1, and NM_001145822.1. The protein is specified by UniProt accession code P07951.

The term “Thioredoxin Reductase 1 (TXNRD1)”, as is used herein, refers to a selenocysteine-containing flavoenzyme, which reduces thioredoxins, as well as other substrates. The gene encoding TXNRD1 is specified by HGNC accession code 12437 and Ensembl accession code ENSG00000198431. Known RefSeq cDNA sequences are provided by GenBank accession numbers NM_001093771.3, NM_001261445.2, NM_001261446.2, NM_003330.4, NM_182729.3, NM_182742.3, and NM_182743.3. The protein is specified by UniProt accession code Q16881.

The term “Vitelline Membrane Outer Layer 1 Homolog (VMO1)”, as is used herein, refers to a protein that localized in extracellular exosomes. The gene encoding VMO1 is specified by HGNC accession code 30387 and Ensembl accession code ENSG00000182853. Known RefSeq cDNA sequences are NM_001144939.2, NM_001144940.2, NM_001144941.2, and NM_182566.3. The protein is specified by UniProt accession code Q7Z5L0.

The term “ACTA2”, as is used herein, refers to actin alpha 2 or alpha smooth muscle actin (α-SMA). The gene encoding ACTA2is specified by HGNC accession code 59 and Ensembl accession code ENSG00000107796. The protein is specified by UniProt accession code P62736.

The term “ALDH1A1”, as is used herein, refers to Aldehyde dehydrogenase 1 family, member A1. The gene encoding ALDH1A1 is specified by HGNC accession code 216 and Ensembl accession code ENSG0000016509. The protein is specified by UniProt accession code P00352.

The term “ATP1A1”, as is used herein, refers to Sodium/potassium-transporting ATPase subunit alpha-1. The gene encoding ATP1A1 is specified by HGNC accession code 476 and Ensembl accession code ENSG00000163399. The protein is specified by UniProt accession code P05023.

The term “CD10”, as is used herein, refers to Membrane Metalloendopeptidase (MME), a cell surface receptor and transmembrane precursor protein. The gene encoding CD10 is specified by HGNC accession code 7154 and Ensembl accession code ENSG00000196549. Known RefSeq cDNA sequences are provided by GenBank accession numbers NM_000902.5, NM_001354642.2, NM_001354643.1, NM_001354644.1, NM_007287.4, NM_007288.3, and NM_007289.4. The protein is specified by UniProt accession code P08473.

The term “CD166”, as is used herein, refers to Activated Leucocyte Cell Adhesion Molecule (ALCAM), the leukocyte cell adhesion molecule that binds to T-cell differentiation antigen CD6. The gene encoding CD166 is specified by HGNC accession code 400 and Ensembl accession code ENSG00000170017. Known RefSeq cDNA sequences are provided by GenBank accession numbers NM_001243280.2, NM_001243281.2, NM_001243283.2, and NM_001627.4. The protein is specified by UniProt accession code Q13740.

The term “CD44”, as is used herein, refers to a cell-surface glycoprotein that is a receptor for hyaluronic acid, CD44 is involved in cell-cell interactions, cell adhesion and migration. The gene encoding CD44 is specified by HGNC accession code 1681 and Ensembl accession code ENSG00000026508. Known RefSeq cDNA sequences are provided by GenBank accession numbers NM_000610.4, NM_001001389.2, NM_001001390.2, NM_001001391.2, NM_001001392.2, NM_001202555.2, NM_001202556.2, and NM_001202557.2. The protein is specified by UniProt accession code P16070.

The term “CD56”, as is used herein, refers to Neural Cell Adhesion Molecule 1 (NCAM1) a cell adhesion protein that is involved in cell-cell interactions as well as cell-matrix interactions. The gene encoding CD56 is specified by HGNC accession code 7656 and Ensembl accession code ENSG00000149294. Known RefSeq cDNA sequences are provided by GenBank accession numbers NM_000615.7, NM_001076682.4, NM_001242607.2, NM_001242608.2, and NM_001386289.1, and 26 other sequences. The protein is specified by UniProt accession code P13591.

The term “CDH2”, as is used herein, refers to Cadherin-2. The gene encoding CDH2 is specified by HGNC accession code 1000 and Ensembl accession code ENSG00000170558. The protein is specified by UniProt accession code P19022.

The term “COL4A3”, as is used herein, refers to Collagen alpha-3(IV) chain. The gene encoding COL4A3 is specified by HGNC accession code 1285 and Ensembl accession code ENSG00000169031. The protein is specified by UniProt accession code Q01955.

The term “PRDX6”, as is used herein, refers to Peroxiredoxin-6. The gene encoding PRDX6 is specified by HGNC accession code 9588 and Ensembl accession code ENSG00000117592. The protein is specified by UniProt accession code P30041.

The term “PITX2”, as is used herein, refers to Paired-like homeodomain transcription factor 2 also known as pituitary homeobox 2. The gene encoding PITX2 is specified by HGNC accession code 5308 and Ensembl accession code ENSG00000164093. The protein is specified by UniProt accession code Q99697.

The term “SLC4A11”, as is used herein, refers to Sodium bicarbonate transporter-like protein 11. The gene encoding SLC4A11 is specified by HGNC accession code 83959 and Ensembl accession code ENSG00000088836. The protein is specified by UniProt accession code Q8NBS3.

Other abbreviations used herein include “CENPF”, for Centromere protein F; “CD105” for Endoglin; “CD133” for prominin-1; “CD24” for Signal transducer CD24; “CD26” for Dipeptidyl peptidase-4 (DPP4), also known as adenosine deaminase complexing protein 2; “CDKN1A” and “CDKN2A” for cyclin-dependent kinase inhibitor 1 or CDK-interacting protein 1 and cyclin-dependent kinase inhibitor 2A, respectively; COL4A1, COL4A2 and COL4A6 for Collagen alpha-1(IV) chain, Collagen alpha-2(IV) chain and Collagen alpha-6(IV) chain, respectively; COL5A2, for Collagen alpha-2(V) chain; “COL6A1” and “COL6A3” for Collagen alpha-1(VI) chain and Collagen alpha-3(VI) chain, respectively; “FBLN5” for Fibulin-5; “GOLGA8A” for Golgin A8 family member A; “KERA” for keratocan; “KRT12” and “KRT14” for Keratin 12 and 14, respectively; “LGALS1” for Galectin-1; “LUM” for Lumican; “MKI67” for Antigen KI-67; “MT2A” for Metallothionein-2; “PAX6” for Paired box protein Pax-6, also known as aniridia type II protein (AN2) or oculorhombin; “PTTG1” for Securin; and “TAGLN” for Transgelin.

The term “antibody”, as is used herein, refers to an antigen binding protein comprising at least a heavy chain variable region (Vh) that binds to a target epitope. The term antibody includes monoclonal antibodies comprising immunoglobulin heavy and light chain molecules, single heavy chain variable domain antibodies, and variants and derivatives thereof, including chimeric variants of single heavy chain variable domain antibodies and multivalent and/or multispecific variants of single heavy chain variable domain antibodies. Said variants and derivatives include fragment antigen-binding (Fab); single chain Fv (scFv) comprising heavy (VH) and light chain (VL) domains; tandem scFv; single chain Fab (scFab); an improved scFab (Koerber et al., 2015. J Mol Biol 427: 576-86), or an antibody mimetic such as a designed ankyrin repeat protein, a binding protein that is based on a Z domain of protein A, a binding protein that is based on a fibronectin type III domain, engineered lipocalin, and a binding protein that is based on a human Fyn SH3 domain (Skerra, 2007. Current Opinion Biotechnol 18: 295-304; Škrlec et al., 2015. Trends Biotechnol 33: 408-418).

The term “antigen”, as is used herein, refers to a biological molecule, usually a protein, peptide, polysaccharide, lipid or conjugate which contains at least one epitope to which an antibody can selectively bind. The term antigen preferably refers to a polypeptide moiety. The term antigen includes reference to at least one, or all, antigenic epitopes of polypeptide moieties or proteins described herein.

The term “Rho kinase inhibitor (ROCK inhibitor)”, as is used herein, refers to an inhibitor of Rho-associated coiled-coil containing kinase. Suitable Rho kinase inhibitors include HA-100 (1-(5-isoquinolinesulfonyl)piperazine hydrochloride), Y-27632 dihydrochloride ((1R,4r)-4-((R)-1-aminoethyl)-N-(pyridin-4-yl)cyclohexanecarboxamide), thiazovivin (N-benzyl-[2-(pyrimidin-4-yl)amino]thiazole-4-carboxamide), DJ4 ((5Z)-2-5-(1H-pyrrolo[2,3-b]pyridine-3-ylmethylene)-1,3-thiazol-4(5H)-one, fasudil (5-(1,4-diazepan-1-ylsulfonyl)isoquinoline), hydroxyfadusil (5-(1,4-diazepan-1-ylsulfonyl)-2H-isoquinolin-1-one); H1152 (4-methyl-5-[[(2S)-2-methyl-1,4-diazepan-1-yl]sulfonyl]isoquinoline; dihydrochloride), netarsudil ([4-[(2S)-3-amino-1-(isoquinolin-6-ylamino)-1-oxopropan-2-yl]phenyl]methyl 2,4-dimethylbenzoate), RKI-1447 (1-[(3-hydroxyphenyl)methyl]-3-(4-pyridin-4-yl-1,3-thiazol-2-yl)urea), 4-ripasudil (fluoro-5-[[(2S)-2-methyl-1,4-diazepan-1-yl]sulfonyl]isoquinoline), Y-33075 (4-(1-aminoethyl)-N-(1H-pyrrolo[2,3-b]pyridin-4-yl)benzamide; hydrochloride), verosudil (AR-12286; 2-(dimethylamino)-N-(1-oxo-2H-isoquinolin-6-yl)-2-thiophen-3-ylacetamide), AT13148 ((S)-1-(4-(1H-pyrazol-4-yl)phenyl)-2-amino-1-(4-chlorophenyl)ethanol), belumosudil (2-[3-[4-(1H-Indazol-5-ylamino)quinazolin-2-yl]phenoxy]-N-propan-2-ylacetamide), chroman 1 ((3S)—N-[2-[2-(dimethylamino)ethoxy]-4-(1H-pyrazol-4-yl)phenyl]-3,4-dihydro-6-methoxy-2H-1-benzopyran-3-carboxamide dihydrochloride), GSK-576371, GSK429286A (N-(6-fluoro-1H-indazol-5-yl)-2-methyl-6-oxo-4-(4-(trifluoromethyl)phenyl)-1,4,5,6-tetrahydropyridine-3-carboxamide), LX-7101 (3-(4-(aminomethyl)-1-(5-methyl-7H-pyrrolo[2,3-d]pyrimidin-4-yl)piperidine-4-carboxamido)phenyl dimethylcarbamate), TCS-7001 (6-chloro-N4-(3,5-difluoro-4-((3-methyl-1H-pyrrolo[2,3-b]pyridin-4-yl)oxy)phenyl)pyrimidine-2,4-diamine), or a combination thereof. An example of a suitable Rho kinase inhibitor is provided by RevitaCell™ Supplement (Gibco™) Typically the amount of a Rho kinase inhibitor will be between about 0.1 to 50 μM, preferably about 1 to 10 μM.

The term “p53 inhibitor”, as is used herein, refers to an inhibitor of a DNA-binding tumor suppressor termed p53. This protein upregulates growth arrest and apoptosis-related genes in response to stress signals, thereby influencing programmed cell death, cell differentiation and cell cycle control mechanisms. A p53 inhibitor may include pifithrin-α (2-(2-imino-4,5,6,7-tetrahydro-1,3-benzothiazol-3-yl)-1-(4-methylphenyl)ethanone; hydrobromide), cyclic pifithrin-alpha hydrobromide (2-(4-methylphenyl)-5,6,7,8-tetrahydroimidazo[2,1-b][1,3]benzothiazole; hydrobromide), pifithrin-mu (2-phenylethynesulfonamide), NSC-66811 (7-[anilino(phenyl)methyl]-2-methylquinolin-8-ol), SL-01 (benzyl [(2S)-4-chloro-3-oxo-1-phenyl-2-butanyl]carbamate, NiCur (3,3′-((1E,1′E)-(2-oxocyclohexane-1,3-diylidene)bis(methaneylylidene)) dibenzonitrile), and MS7972 (9-acetyl-3,4-dihydro-2H-carbazol-1-one). Typically the amount of a p53 inhibitor will be between about 0.01 to 50 μM, preferably about 0.5 to 10 μM.

### Methods of Isolating and Cultivating CECs

Methods of the invention include the generation of a population of cultured CECs. Said cell population preferably comprises human CECs (hCECs), preferably native-like hCECs. Said cell population may be produced by culturing primary CECs for a limited number of passages. Said primary CECs may be obtained from an individual having said corneal endothelial dysfunction, and implanted back after expansion by cultivation into the same individual (autologous transplant), or may be obtained from a healthy donor and transplanted (optionally after expansion by cultivation) to an individual having a corneal endothelial dysfunction (allogeneic transplant). As an alternative, said CECs may be obtained from stem cells such as human pluripotent stem cells (hPSC), including human induced pluripotent stem cells (iPSCs). Methods for generating CECs using such stem cells are well known in the art (McCabe et al., 2015. PLoS One 10: e0145266; Song et al., 2016. Exp Eye Res 151: 107-114; Wagoner et al., 2018. Biol Open 7: bio032102; Zhang et al., 2014. Stem Cells Dev 23: 1340-1354).

Methods of the invention include the generation of CECs from stem cells such as human stem cells, including human pluripotent stem cells (PSCs). For this, PSCs, preferably human PSCs such as H9 (Amit et al., 2000. Developm Biol 227: 271-278) or Regea08/017 (Skottman, 2010. In Vitro Cell Dev Biol-Animal 46: 206-209), or induced pluripotent stem cell (iPSC), may be cultured under feeder-free and serum-free conditions in the presence of a fibroblast growth factor and small molecule inhibitors, including ALK inhibitors such as SB431542 and/or LDN193189, and a Wnt-production inhibitor such as IWP2, to become restricted to eye field stem cells (EFSCs), as described (Zhao and Afshari, 2016. Invest Ophthalmol Vis Sci 57: 6878-6884). EFSCs may be cultured in the presence of crest induction medium (e.g. STEMCELL Technologies, Vancouver, Canada) to generate ocular neural crest stem cell (NCSC), which in turn may be differentiated into CECs, as described in the published International Patent Application WO2017/190136A).

Native-like hCECs may be generated from pluripotent human stem cells in the presence of a transforming growth factor (TGF) beta receptor inhibitor such as SB431542, a GSK-3-specific inhibitor such as CHIR99021 and retinoic acid, as described (Grönroos et al., 2021. Cells 10: 331). Cells may be grown on an extracellular matrix, for example a human laminin such as recombinant LN521™ (Biolamina, Sundbyberg, Sweden).

In addition, native-like hCECs may be generated from human induced pluripotent stem cells in the presence of a transforming growth factor (TGF) beta receptor inhibitor such as SB431542, a GSK-3-specific inhibitor such as CHIR99021 (Wagoner et al., 2018. Biology Open 7: bio032102).

Methods of the invention also involve the isolation of corneal endothelial tissue from a donor, followed by the dissociation of said tissue to obtain CECs, and the propagation of said cells to multiply the number of CECs. A donor preferably has no history of transmittable infection such as human immunodeficiency virus (HIV), or cornea endothelial pathology, and no active cancer.

The cornea serves as a barrier against particles that can harm an eye and filters out some amounts of ultraviolet light. Cornea is composed of three layers termed epithelium, stroma and endothelium. The endothelium is a single layer of cells that works as a pump, expelling excess water as it is absorbed into the stroma.

All or part of a cornea may be excised from a donor by employing lamellar keratoplasty such as full-thickness penetrating keratoplasty, posterior lamellar keratoplasty, deep lamellar endothelial keratoplasty, Descemet's Stripping Endothelial Keratoplasty (DSEK) or Descemet's Membrane Endothelial Keratoplasty (DMEK).

In an embodiment, a part of a cornea comprising endothelial cells may be isolated, for example by employing DSEK or DMEK. For this, a section of the corneal tissue that is excised, comprising the endothelial layer, Descemet's membrane, and part of the stroma, may be separated from the rest of the tissue. Said separation may be performed by hand, or with an automated microkeratome.

As an alternative, a part of an excised corneal tissue may be isolated by a punch, such as a corneal punch. The endothelial cells may be isolated from the punched material, for example by lifting using a DMEK cleavage hook, and stripping of the corneal endothelium using a forceps, such as a McPherson Suture Tying Forceps.

The excised corneal tissue may be incubated with a protease such as collagenase, preferably collagenase A (for example, Roche Applied Science, Mannheim, Germany), and incubated in a tissue culture dish. To enrich for endothelial cells, incubation may be performed in the presence of a selective L-valine-free medium, which inhibited fibroblast growth. Fibronectin, collagen and, optionally, also albumin, may function as a substrate for endothelial cells. Incubation preferably is performed in the presence of serum-free media, comprising nutritional and hormonal formulations that allow for cells to be cultured without the use of animal sera. If needed to generate single cells, small clumps of corneal endothelial cells may be incubated with a protease such as trypsin or an alternative therefore such as TrypLE™ select or express reagent (Thermo Fisher Scientific) or Accutase® (BD Bioscience, Franklin Lakes, NJ USA).

In order to propagate or expand CECs in culture, CECs may be incubated in different media at different stages of their cultivation, for example in a serum-free medium, Ham's F12 medium, or M199 medium, or mixtures thereof such as a 1:1 mixture of Ham's F12 medium and M199 medium. Said media may all commercially be obtained, for example from Thermo Fisher Scientific, or from Sigma-Aldrich. Said media optionally comprise antibiotics, such as penicillin, streptomycin, and/or amphotericin B, and may be supplemented with serum, for example with 5% fetal bovine serum. Said media may comprise a water soluble anti-oxidant such as, for example, ascorbic acid and supplements such as vitamins, amino acids, insulin, transferrin, selenite, ethanolamine, spermine, putrescine, and lipids including fatty acids such as linoleic acid. Said media may further comprise a Rho kinase inhibitor such as RevitaCell™ Supplement or Y-27632. Media preferably are refreshed every other day. One passage of cells normally takes about 15-17 days of culture until reaching about confluency. Passaging of cells to a fresh tissue culture dish may be performed at a seeding density of 5000-20,000 cells/cm², such as 7000-15,000 cells/cm², including about 10,000 cells/cm² in medium. All cell culturing may be performed in incubators at standard conditions, such as a humidified atmosphere of 37° C. and 5% CO₂.

For example, hCECs may be harvested, seeded and allowed to attach in Ham's F12/M199 (1:1), 5% fetal bovine serum, supplemented with 20 mg/ml ascorbic acid, 1% Insulin-Transferrin-Selenium (ITS: Thermo Fisher Scientific), 10 ng/ml bovine Fibroblast Growth Factor (R&D Systems, Minneapolis, MN, USA), and 1% antibiotic/antimycotic. This first step is to stabilize isolated hCECs, which may take a period of time, for example between 12 hours and 48 hours, such as about 16 hours or overnight. To promote proliferation of the adhered CECs, human endothelial-serum-free medium (Thermo Fisher Scientific), supplemented with 5% FBS and 1% antibiotic/antimycotic may be used. In a final step, for example, when hCECs become approximately 80% confluent, medium may be replaced by Ham's F12/M199 (1:1), 5% fetal bovine serum, supplemented with 20 mg/ml ascorbic acid, 1% Insulin-Transferrin-Selenium (ITS: Thermo Fisher Scientific), 10 ng/ml bovine Fibroblast Growth Factor (R&D Systems, Minneapolis, MN, USA), to stabilize and maintain the expanded hCECs, as described (Peh et al., 2015. Cell Transplant 24: 287-304). Hereafter, confluent hCECs may be dissociated using a protease such as trypsine or TrypLE™ (Thermo Fisher Scientific) and seeded at a density of about 1×10⁴ cells per cm², and subjected to the same steps of stabilization, proliferation and stabilization/maintenance to generate further passages of hCECs.

### Methods of Determining the Quality of CECs and Methods of Selecting CECs

Described herein are methods of determining the quality of CECs and methods of selecting CECs, such as human CECs, comprising the step of measuring in or on a cultured CEC a level of expression of a gene as indicated herein. In aspects of this invention, the quality assessment or selection is based on a level of expression of at least one marker selected from CGNL1, VMO1, THBS2, APP, S100A10, TXNRD1, TPM2, KCNE4, PDGFRA, and HPPD, optionally further including CD56, CD166, CD44 and/or CD10. Quality assessment or selection may additionally be based on morphological characteristics of the cultured CECs, e.g. on the basis of cell shape such as a cell with a polygonal shape versus a cell that is elongated or even spindle-like shape.

Determining the level of expression of a marker as defined herein may occur through detection of the gene expression product, such as the (m)RNA or protein product. In embodiments of this invention, the level of RNA expression may be determined by RNAseq, such as scRNAseq as described herein. Alternatively, the level of RNA expression may be determined by any method available to one of skill in the art for determining the amount of specific RNA molecules in cells or cell populations. For instance, use may be made of Fluorescence in-situ hybridization (FISH), reverse transcription-polymerase chain reaction (RT-PCR), reverse transcription loop-mediated isothermal amplification (RT-LAMP), and TaqMan-based real-time RT-PCR, and Northern Blot analysis. The level of expression may also be determined by any method available to one of skill in the art for determining the amount of specific protein molecules in cells or cell populations, such as Western Blot analysis or proteomics techniques including as mass spectrometry such as LC-MS/MS. Preferably, protein expression is determined by using immunocytochemical (ICC) techniques known in the art, which techniques refers to immunostaining of cultured cell lines or primary cells and offers a semi-quantitative means of analyzing the relative abundance, conformation, and subcellular localization of target antigens. ICC techniques may use chromogenic detection in which enzyme conjugated antibodies convert chromogen substrates to a colored precipitate at the reaction site. Alternatively, immunocytochemistry/immunofluorescence (ICC/IF) assays may be used, wherein the cellular antigens are visualized using either fluorochrome-conjugated primary antibodies (direct detection) or a two-step method (indirect detection) involving an unlabeled primary antibody followed by a fluorochrome-conjugated secondary antibody. By combining different fluorochrome-labeled antibodies, multiplex ICC/IF can detect several antigens in the same sample. A standard ICC/IF protocol involves fixation, permeabilization, blocking, immunolabeling, counterstaining, and microscopic imaging or flow cytometric analysis of stained cells. It is well within the capabilities of one of ordinary skill in the art to determine the level of expression of at least one marker as defined herein using the technology available. Flow cytometry, as an alternative, or in addition to fluorescence microscopy, is a useful tool for measuring expression of cell surface, cytoplasmic and nuclear protein markers. All markers herein may in principle be combined with cell sorting of viable cells by flow cytometry (FACS).

A level of expression of at least one marker selected from CGNL1, VMO1, THBS2, APP, S100A10, TXNRD1, TPM2, KCNE4, PDGFRA, and HPPD, optionally further including CD56, CD166, CD44 and/or CD10, may be used to assess the quality of a cell population comprising CECs, such as hCECs. A level of expression of at least one of CGNL1, VMO1, THBS2, APP, S100A10, TXNRD1, TPM2, KCNE4, PDGFRA, and HPPD in or on individual CECs may help to determine whether a population of cultured CECs comprises native-like CECs, and/or to determine the incidence of native-like CECs in a population of cultured CECs.

Quality assessment or selection is preferably based on distinguishing native-like CECs from non-native-like CECs. Such a distinction may be useful for selecting native-like CECs and separating native-like CECs from non-native-like CECs and enriching populations for native-like CECs. The selection and separation steps may be performed successively or simultaneously. For instance, selection and separation steps may be performed by use of antigen-based cell sorting such as fluorescence activated cell sorting (FACS) or by use of immunomagnetic cell sorting (IMCS) using one or more antibodies that recognize (an epitope on) one or more of the markers described herein. As an alternative, or in addition, quality assessment or selection between CECS may be performed by determining mRNA expression levels of one or more of CGNL1, VMO1, THBS2, APP, S100A10, TXNRD1, TPM2, KCNE4, PDGFRA, and HPPD, optionally further including CD56, CD166, CD44 and/or CD10. An mRNA expression level of one or more of CGNL1, VMO1, THBS2, APP, S100A10, TXNRD1, TPM2, KCNE4, PDGFRA, and HPPD, optionally further including CD56, CD166, CD44 and/or CD10, may be determined by any means known in the art such as microarray analysis, fluorescence in-situ hybridization (FISH), quantitative PCR (qPCR), or RNA-sequencing (RNA-seq). The expression levels of multiple marker genes are preferably assessed simultaneously, for example by multicolor FISH, multiplex qPCR, microarray analysis or RNA-seq. Most preferably the expression levels of marker genes is assessed by use of marker protein-specific antibodies.

Methods of measuring in or on a cultured CEC a level of expression of a marker gene as indicated herein may be disruptive or non-disruptive. Disruptive methods may require lysis of the cells. Methods for measuring mRNA expression levels may for instance be disruptive. When using disruptive methods of measuring marker gene expression levels, said methods may be employed on an aliquot of a culture or population of CECs, as an assessment of the quality of the whole culture or population of CECs. The level of expression may be determined on a multitude of cells from the culture or population of CEC, and may be determined as an average over a multitude of cells, or on a per cell basis.

A reference level of expression may be used to determine whether a cell represents a CGNL1+, VMO1−, THBS2−, APP+, S100A10−, TXNRD1−, TPM2−, KCNE4−, PDGFRA−, and HPPD− phenotype. As an alternative, a threshold may be determined for each of CGNL1, VMO1, THBS2, APP, S100A10, TXNRD1, TPM2, KCNE4, PDGFRA, and HPPD, optionally further including CD56, CD166, CD44 and/or CD10, in order to determine whether a cell represents a CGNL1+, VMO1−, THBS2−, APP+, S100A10−, TXNRD1−, TPM2−, KCNE4−, PDGFRA−, and HPPD− phenotype. As is known to a person skilled in the art, said threshold will depend on the individual marker, the method that is used, and optionally also in the instrumentation that is used.

The determined mRNA expression level can be normalized for differences in the total amounts of mRNA expression products between two separate batches of CECs by comparing the level of expression of one or more genes that are presumed not to differ in expression level between samples such as glyceraldehyde-3-phosphate-dehydro-genase, ß-actin, and ubiquitin. Conventional methods for normalization of array data include global analysis, which is based on the assumption that the majority of genetic markers on an array are not differentially expressed between samples (Yang et al., 2002. Nucl Acids Res 30: 15). Alternatively, an array may comprise specific probes that are used for normalization. These probes preferably detect mRNA products from housekeeping genes such as glycer-aldehyde-3-phosphate dehydrogenase and 18S rRNA levels, of which the RNA level is thought to be constant in a given cell and independent from the differentiation stage or prognosis of said cell. Similarly, qPCR may include primers and probes to detect mRNA products from housekeeping genes such as glycer-aldehyde-3-phosphate dehydrogenase and 18S rRNA levels. Normalization of RNAseq data may involve determining a global scaling method such as Reads per Kilobase Million (RPKM), Fragments per Kilobase Million (FPKM) or Transcripts per Million (TPM).

Methods for determining an mRNA expression level of one or more of CGNL1, VMO1, THBS2, APP, S100A10, TXNRD1, TPM2, KCNE4, PDGFRA, and HPPD, optionally further including CD56, CD166, CD44 and/or CD10 may require the conversion of mRNA into cDNA and/or cRNA using a reverse-transcriptase enzyme such as M-MLV reverse-transcriptase from Moloney murine leukemia virus, or AMV reverse-transcriptase from avian myeloblastosis virus, as is known to a person skilled in the art. Kits and protocols for reverse transcribing mRNA can be commercially obtained, e.g. from Thermo Fisher Scientific, New England Biolabs (Ipswich, MA, USA) and Stratagene (La Jolla, CA, USA). Microarray analysis involves the use of selected probes that are immobilized on a solid surface, an array. Said probes are able to hybridize to gene expression products such as mRNA, or derivates thereof such as cDNA. The probes are exposed to labeled sample gene expression products, or labelled derivates thereof, hybridized, washed, where after the abundance of gene expression products or derivates thereof in the sample that are complementary to a probe is determined by determining the amount of label that remains associated to a probe. The probes on a microarray may comprise DNA sequences, RNA sequences, or copolymer sequences of DNA and RNA. The probes may also comprise DNA and/or RNA analogues such as, for example, nucleotide analogues or peptide nucleic acid molecules (PNA), or combinations thereof. The sequences of the probes may be full or partial fragments of genomic DNA. The sequences may also be in vitro synthesized nucleotide sequences, such as synthetic oligonucleotide sequences.

In the context of the invention, a probe preferably is specific for a gene expression product of a gene selected from CGNL1, VMO1, THBS2, APP, S100A10, TXNRD1, TPM2, KCNE4, PDGFRA, and HPPD, optionally further including CD56, CD166, CD44 and/or CD10. A probe is specific when it comprises a continuous stretch of nucleotides that is complementary, over the whole length of the stretch, to a nucleotide sequence of a gene expression product, or a cDNA product thereof. A probe can also be specific when it comprises a continuous stretch of nucleotides that are partially complementary to a nucleotide sequence of a gene expression product of said gene, or a cDNA product thereof. Partially means that a maximum of 5 nucleotides, more preferable 4 nucleotides, more preferable 3 nucleotides, more preferable 2 nucleotides and most preferable one nucleotide differs from the corresponding nucleotide sequence of a gene expression product of said gene. The term complementary is known in the art and refers to a sequence that is related by base-pairing rules to the sequence that is to be detected. It is preferred that the sequence of the probe is carefully designed to minimize nonspecific hybridization to said probe.

The specificity of probe is further determined by the hybridization and/or washing conditions. The hybridization and/or washing conditions are preferably stringent, which are determined by inter alia the temperature and salt concentration of the hybridization and washing conditions, as is known to a person skilled in the art. An increased stringency will substantially reduce non-specific hybridization to a probe, while specific hybridization is not substantially reduced. Stringent conditions include, for example, washing steps for five minutes at room temperature 0.1× sodium chloride-sodium citrate buffer (SSC)/0.005% Triton X-102. More stringent conditions include washing steps at elevated temperatures, such as 37° Celsius, 45° Celsius, or 65° Celsius, either or not combined with a reduction in ionic strength of the buffer to 0.05×SSC or even 0.01×SSC, as is known to a skilled person.

It is preferred that the probe is, or mimics, a single stranded nucleic acid molecule. The length of a probe can vary between 15 bases and several kilo bases, and is preferably between 20 bases and 1 kilobase, more preferred between 25 and 100 bases, such as 60 nucleotides. Said probe is preferably identical over the whole length to a nucleotide sequence of a gene expression product of a gene, or a cDNA product thereof. In a method of the invention, probes comprising probe sequences as indicated in Table 1 can be employed.

To determine an RNA expression level by micro arraying, gene expression products in the sample are preferably labeled, either directly or indirectly, and contacted with probes on the array under conditions that favor duplex formation between a probe and a complementary molecule in the labeled gene expression product sample. The amount of label that remains associated with a probe after washing of the microarray can be determined and is used as a measure for the gene expression level of a nucleic acid molecule that is complementary to said probe.

Image acquisition and data analysis can subsequently be performed to produce an image of the surface of the hybridized array. For this, the array may be dried and placed into a laser scanner to determine the amount of labeled sample that is bound to a probe at a predetermined spot. Laser excitation will yield an emission with characteristic spectra that is indicative of the labelled sample that is hybridized to a probe molecule.

An array preferably comprises multiple spots encompassing a specific probe. A probe preferably is present in duplicate, in triplicate, in quadruplicate, in quintuplicate, in sextuplicate or in octuplicate on an array. The multiple spots preferably are at randomized opposition on an array to minimize bias. The amount of label that remains associated with the probe at each spot may be averaged, where after the averaged level can be used as a measure for the gene expression level of a nucleic acid molecule that is complementary to said probe. In addition, a gene product may be hybridized to two or more different probes that are specific for that gene product.

A further method for determining RNA expression levels is sequencing, preferably next-generation sequencing (NGS), of RNA samples, with or without prior amplification of the RNA expression products. NGS, or high throughput sequencing, techniques for sequencing RNA, termed RNA-seq, have been developed. NGS platforms, including Illumina® sequencing; Roche 454 Pyrosequencing®, ion torrent and ion proton sequencing, and ABI SOLiD® sequencing, allow sequencing of fragments of DNA in parallel. Bioinformatics analyses are used to piece together these fragments by mapping the individual reads. Each base is sequenced multiple times, providing high depth to deliver accurate data and an insight into unexpected DNA variation. NGS can be used to sequence a complete exome including all or small numbers of individual genes.

Pyrosequencing detects the release of inorganic pyrophosphate (PPi) as particular nucleotides are incorporated into the nascent strand (Ronaghi et al., 1996. Analytical Biochemistry 242: 84-9; Ronaghi, 2001. Genome Res 11: 3-11; Ronaghi et al., 1998. Science 281: 363; U.S. Pat. Nos. 6,210,891; 6,258,568; and 6,274,320, which are all incorporated herein by reference. In pyrosequencing, released PPi can be detected by being immediately conversion to adenosine triphosphate (ATP) by ATP sulfurylase, and the level of ATP generated is detected via luciferase-produced photons.

NGS also includes so called third generation sequencing platforms, for example nanopore sequencing on an Oxford Nanopore Technologies platform, and single-molecule real-time sequencing (SMRT sequencing) on a PacBio platform, with or without prior amplification of the RNA expression products.

Said high-throughput sequencing methods may be used for single-cell RNA-sequencing (scRNA-seq). For this technique, single cells are isolated, followed by the generation of sequencing libraries for mRNA isolated from each single cell. Methods and means for scRNA-seq are commercially available, for example RNAdia 2.0 and Nadia Instrument (Dolomite Bio, Royston, UK), and GEXSCOPE® Single Cell RNA Library Kit (Singleron Biotechnologies, Chongqing, China), which may be combined with instruments such as BaseSpace™ Sequence Hub from Illumina® (San Diego, CA, USA).

Quantitative PCR (qPCR), or real-time PCR (RT-PCR), is a technique that is used to amplify and simultaneously quantify a template nucleic acid molecule such as an RNA. The detection of the amplified product can in principle be accomplished by any suitable method known in the art. The amplified double stranded (ds) DNA product may be directly stained or labelled with a radioactive label, an antibody, a luminescent dye, a fluorescent dye, or an enzyme reagent. Direct DNA stains include for example intercalating dyes such as acridine orange, ethidium bromide, ethidium monoazide or Hoechst dyes. These intercalating dyes are non-specific and bind to all ds DNA in a PCR reaction. An increase in ds DNA products during amplification results in an increased fluorescence intensity being measured.

Another method for direct DNA detection or for mRNA detection includes the use of a sequence specific DNA or RNA probe. Said probe may be labelled, for example with a fluorescent dye, or include a fluorescent reporter and quencher. Upon binding of the probe to its complementary sequence, the proximity of the reporter and the quencher is broken, resulting in the emission of fluorescence. Commonly used reporter dyes include FAM (Applied Biosystems), HEX (Applied Biosystems), ROX (Applied Biosystems), YAK (ELITech Group) and VIC (Life Technologies) and commonly used quenchers include TAMRA (Applied Biosystems), BHQ (Biosearch Technologies) and ZEN (Integrated DNA Technologies).

Alternatively, the amplified product may be detected by incorporation of labelled dNTP bases into the synthesized DNA fragments. Detection labels which may be associated with nucleotide bases include, for example, fluorescein, cyanine dye and 5-bromo-2′-deoxyuridine (BrdU).

For the simultaneous detection of multiple nucleic acid gene expression products, a multiplex qPCR can be used. In multiplex qPCRs, two or more template nucleic acid molecules are amplified and quantified in the same reaction. A commonly used method of achieving the simultaneously detection of multiple targets, is by using probes with different fluorescent dyes to distinguish distinct nucleic acid targets.

QPCR instruments and software are commercially available, for example StepOnePlus™ (Thermo Fisher Scientific), AriaDx™ and AriaMx™ qPCR (Agilent, Santa Clara, CA USA), and Q (Quantabio, Beverly, MA, USA).

Quality assessment, selection and separation steps may for instance be performed following expansion of CECs in culture. For instance, at one or more of the passages of cultivation, preferably at the end of culturing, native-like CECs may be distinguished from non-native-like CECs by determining a level of expression of at least one of CGNL1, VMO1, THBS2, APP, S100A10, TXNRD1, TPM2, KCNE4, PDGFRA, and HPPD in or on an individual CEC, whereby a native-like CEC represents a CGNL1+, VMO1−, THBS2−, APP+, S100A10−, TXNRD1−, TPM2−, KCNE4−, PDGFRA−, and HPPD− phenotype, when compared to a reference such as a non-native-like CEC. In addition, a level of expression of CD56, CD166, CD44 and CD10 maybe determined, whereby a native-like CEC represents a CD56+, CD166+, CD44− and CD10− phenotype, when compared to a reference such as a non-native-like CEC. Further useful markers in aspects of the invention include SLC4A11, PRDX6, SLC4A11, PITX2, ATP1A1, COL4A3, CDH2, CD105, CD24, CD26, and CD133.

The methods of the invention allow routine culturing and expanding of native-like CECs until confluency by the second passage. This means that CECs isolated from a single donor can be expanded to 5×10⁶ to 10×10⁶ cells, meaning that 5-10 patients may be treated with by methods of treatment according to the present invention.

Quality assessment, selection and separation of native-like from non-native-like cultured CECs is preferably performed by antigen-based cell sorting such as fluorescence activated cell sorting (FACS) or immunomagnetic cell sorting (IMCS) using one or more antibodies that recognize an epitope on one or more of CGNL1, VMO1, THBS2, APP, S100A10, TXNRD1, TPM2, KCNE4, PDGFRA, and HPPD, optionally further including CD56, CD166, CD44 and/or CD10.

FACS or IMCS may be used to specifically select or sort native-like CECs by selecting or sorting for cells having a CGNL1+, APP+, CD56+, and/or CD166+ phenotype. As an alternative, FACS or IMCS may be used to specifically select or sort non-native-like CECs by selecting or sorting and excluding cells having a VMO1+, THBS2+, S100A10+, TXNRD1+, TPM2+, KCNE4+, PDGFRA+, and/or HPPD+ phenotype, optionally further excluding cells with a CD44+ and/or CD10+ phenotype. Additionally, said antigen-based cell sorting in order to separate native-like from non-native-like CECs is performed on the basis of (i) the presence of a marker selected from CGNL1+, APP+ and, optionally, CD56+, and/or CD166+, in combination with the absence of a marker selected from VMO1, THBS2, S100A10, TXNRD1, TPM2, KCNE4, PDGFRA, and/or HPPD, optionally further excluding cells with a CD44+ and/or CD10+ phenotype. Furthermore, said antigen-based cell sorting in order to separate native-like CECs from non-native-like CECs may be performed on the basis of one, two, three or more markers, four or more markers, five or more markers, six or more markers, seven or more markers, eight or more markers, nine or more markers, or all markers selected from CGNL1, VMO1, THBS2, APP, S100A10, TXNRD1, TPM2, KCNE4, PDGFRA, and HPPD, optionally further including CD56, CD166, CD44 and/or CD10.

Preferred combinations of markers include CGNL1 and VMO1; CGNL1 and THBS2; CGNL1 and APP; CGNL1 and S100A10; CGNL1 and TXNRD1; CGNL1 and TPM2; CGNL1 and KCNE4; CGNL1 and PDGFRA; CGNL1 and HPPD; VMO1 and THBS2; VMO1 and APP; VMO1 and S100A10; VMO1 and TXNRD1; VMO1 and TPM2; VMO1 and KCNE4; VMO1 and PDGFRA; VMO1 and HPPD; THBS2 and APP; THBS2 and S100A10; THBS2 and TXNRD1; THBS2 and TPM2; THBS2 and KCNE4; THBS2 and PDGFRA; THBS2 and HPPD; APP and S100A10; APP and TXNRD1; APP and TPM2; APP and KCNE4; APP and PDGFRA; APP and HPPD; S100A10 and TXNRD1; S100A10 and TPM2; S100A10 and KCNE4; S100A10 and PDGFRA; S100A10 and HPPD; TXNRD1 and TPM2; TXNRD1 and KCNE4; TXNRD1 and PDGFRA; TXNRD1 and HPPD; TPM2 and KCNE4; TPM2 and PDGFRA; TPM2 and HPPD; KCNE4 and PDGFRA; KCNE4 and HPPD; PDGFRA and HPPD; and optionally at least one of CD56, CD166, CD44 and CD10; CD56 and CD166; CD56 and CD44; CD56 and CD10; CD166 and CD44; CD166 and CD10; CD56, CD166 and CD44; CD56, CD166 and CD10; and CD56, CD166, CD44 or CD10.

Preferred combinations of 3 markers include CGNL1, VMO1 and THBS2; CGNL1, VMO1 and APP; CGNL1, VMO1 and S100A10; CGNL1, VMO1 and TXNRD1; CGNL1, VMO1 and TPM2; CGNL1, VMO1 and KCNE4; CGNL1, VMO1 and PDGFRA; CGNL1, VMO1 and HPPD; CGNL1, THBS2 and APP; CGNL1, THBS2 and S100A10; CGNL1, THBS2 and TXNRD1; CGNL1, THBS2 and TPM2; CGNL1, THBS2 and KCNE4; CGNL1, THBS2 and PDGFRA; CGNL1, THBS2 and HPPD; CGNL1, APP and S100A10; CGNL1, APP and TXNRD1; CGNL1, APP and TPM2; CGNL1, APP and KCNE4; CGNL1, APP and PDGFRA; CGNL1, APP and HPPD; CGNL1, S100A10 and TXNRD1; CGNL1, S100A10 and TPM2; CGNL1, S100A10 and KCNE4; CGNL1, S100A10 and PDGFRA; CGNL1, S100A10 and HPPD; CGNL1, TXNRD1 and TPM2; CGNL1, TXNRD1 and KCNE4; CGNL1, TXNRD1 and PDGFRA; CGNL1, TXNRD1 and HPPD; CGNL1, TPM2 and KCNE4; CGNL1, TPM2 and PDGFRA; CGNL1, TPM2 and HPPD; CGNL1, KCNE4 and PDGFRA; CGNL1, KCNE4 and HPPD; CGNL1, PDGFRA, and HPPD; VMO1, THBS2 and APP; VMO1, THBS2 and S100A10; VMO1, THBS2 and TXNRD1; VMO1, THBS2 and TPM2; VMO1, THBS2 and KCNE4; VMO1, THBS2 and PDGFRA; VMO1, THBS2 and HPPD; VMO1, APP and S100A10; VMO1, APP and TXNRD1; VMO1, APP and TPM2; VMO1, APP and KCNE4; VMO1, APP and PDGFRA; VMO1, APP and HPPD; VMO1, S100A10 and TXNRD1; VMO1, S100A10 and TPM2; VMO1, S100A10 and KCNE4; VMO1, S100A10 and PDGFRA; VMO1, S100A10 and HPPD; VMO1, TXNRD1 and TPM2; VMO1, TXNRD1 and KCNE4; VMO1, TXNRD1 and PDGFRA; VMO1, TXNRD1 and HPPD; VMO1, TPM2 and KCNE4; VMO1, TPM2 and PDGFRA; and VMO1, TPM2 and HPPD; VMO1, KCNE4 and PDGFRA; VMO1, KCNE4 and HPPD; VMO1, PDGFRA, and HPPD; THBS2, APP and S100A10; THBS2, APP and TXNRD1; THBS2, APP and TPM2; THBS2, APP and KCNE4; THBS2, APP and PDGFRA; THBS2, APP and HPPD; THBS2, S100A10 and TXNRD1; THBS2, S100A10 and TPM2; THBS2, S100A10 and KCNE4; THBS2, S100A10 and PDGFRA; THBS2, S100A10 and HPPD; THBS2, TXNRD1 and TPM2; THBS2, TXNRD1 and KCNE4; THBS2, TXNRD1 and PDGFRA; THBS2, TXNRD1 and HPPD; THBS2, TPM2 and KCNE4; THBS2, TPM2 and PDGFRA; THBS2, TPM2 and HPPD; THBS2, KCNE4 and PDGFRA; THBS2, KCNE4 and HPPD; THBS2, PDGFRA, and HPPD; APP, S100A10 and TXNRD1; APP, S100A10 and TPM2; APP, S100A10 and KCNE4; APP, S100A10 and PDGFRA; APP, S100A10 and HPPD; APP, TXNRD1 and TPM2; APP, TXNRD1 and KCNE4; APP, TXNRD1 and PDGFRA; APP, TXNRD1 and HPPD; APP, TPM2 and KCNE4; APP, TPM2 and PDGFRA; APP, TPM2 and HPPD; APP, KCNE4 and PDGFRA; APP, KCNE4 and HPPD; APP, PDGFRA, and HPPD; S100A10, TXNRD1 and TPM2; S100A10, TXNRD1 and KCNE4; S100A10, TXNRD1 and PDGFRA; S100A10, TXNRD1 and HPPD; S100A10, TPM2 and KCNE4; S100A10, TPM2 and PDGFRA; and S100A10, TPM2 and HPPD; S100A10, KCNE4 and PDGFRA; S100A10, KCNE4 and HPPD; S100A10, PDGFRA, and HPPD; TXNRD1, TPM2 and KCNE4; TXNRD1, TPM2 and PDGFRA; TXNRD1, TPM2 and HPPD; TXNRD1, KCNE4 and PDGFRA; TXNRD1, KCNE4 and HPPD; TXNRD1, PDGFRA, and HPPD; TPM2, KCNE4 and PDGFRA; TPM2, KCNE4 and HPPD; TPM2, PDGFRA, and HPPD; KCNE4, PDGFRA, and HPPD; and optionally at least one of CD56, CD166, CD44 and CD10; CD56 and CD166; CD56 and CD44; CD56 and CD10; CD166 and CD44; CD166 and CD10; CD56, CD166 and CD44; CD56, CD166 and CD10; and CD56, CD166, CD44 and CD10.

Preferred combinations of 4 markers include CGNL1, VMO1, THBS2, and APP; CGNL1, VMO1, THBS2, and S100A10; CGNL1, VMO1, THBS2, and TXNRD1; CGNL1, VMO1, THBS2, and TPM2; CGNL1, VMO1, THBS2, and KCNE4; CGNL1, VMO1, THBS2, and PDGFRA; CGNL1, VMO1, THBS2, and HPPD; CGNL1, VMO1, APP, and S100A10; CGNL1, VMO1, APP, and TXNRD1; CGNL1, VMO1, APP, and TPM2; CGNL1, VMO1, APP, and KCNE4; CGNL1, VMO1, APP, and PDGFRA; CGNL1, VMO1, APP, and HPPD; CGNL1, VMO1, S100A10, and TXNRD1; CGNL1, VMO1, S100A10, and TPM2; CGNL1, VMO1, S100A10, and KCNE4; CGNL1, VMO1, S100A10, and PDGFRA; CGNL1, VMO1, S100A10, and HPPD; CGNL1, VMO1, TXNRD1, and TPM2; CGNL1, VMO1, TXNRD1, and KCNE4; CGNL1, VMO1, TXNRD1, and PDGFRA; CGNL1, VMO1, TXNRD1, and HPPD; CGNL1, VMO1, PDGFRA, and TPM2; CGNL1, VMO1, PDGFRA, and KCNE4; CGNL1, VMO1, PDGFRA, and HPPD; CGNL1, VMO1, THBS2, and APP; CGNL1, VMO1, THBS2, and S100A10; CGNL1, VMO1, THBS2, and TXNRD1; CGNL1, VMO1, THBS2, and TPM2; CGNL1, VMO1, THBS2, and KCNE4; CGNL1, VMO1, THBS2, and PDGFRA; CGNL1, VMO1, THBS2, and HPPD; CGNL1, VMO1, APP, and S100A10; CGNL1, VMO1, APP, and TXNRD1; CGNL1, VMO1, APP, and TPM2; CGNL1, VMO1, APP, and KCNE4; CGNL1, VMO1, APP, and PDGFRA; CGNL1, VMO1, APP, and HPPD; CGNL1, VMO1, S100A10, and TXNRD1; CGNL1, VMO1, S100A10, and TPM2; CGNL1, VMO1, S100A10, and KCNE4; CGNL1, VMO1, S100A10, and PDGFRA; CGNL1, VMO1, S100A10, and HPPD; CGNL1, VMO1, TXNRD1, and TPM2; CGNL1, VMO1, TXNRD1, and KCNE4; CGNL1, VMO1, TXNRD1, and PDGFRA; CGNL1, VMO1, TXNRD1, and HPPD; CGNL1, VMO1, PDGFRA, and TPM2; CGNL1, VMO1, PDGFRA, and KCNE4; CGNL1, VMO1, PDGFRA, and HPPD; CGNL1, THBS2, APP, and S100A10; CGNL1, THBS2, APP, and TXNRD1; CGNL1, THBS2, APP, and TPM2; CGNL1, THBS2, APP, and KCNE4; CGNL1, THBS2, APP, and PDGFRA; CGNL1, THBS2, APP, and HPPD; CGNL1, THBS2, S100A10, and TXNRD1; CGNL1, THBS2, S100A10, and TPM2; CGNL1, THBS2, S100A10, and KCNE4; CGNL1, THBS2, S100A10, and PDGFRA; CGNL1, THBS2, S100A10, and HPPD; CGNL1, THBS2, TXNRD1, and TPM2; CGNL1, THBS2, TXNRD1, and KCNE4; CGNL1, THBS2, TXNRD1, and PDGFRA; CGNL1, THBS2, TXNRD1, and HPPD; CGNL1, THBS2, PDGFRA, and TPM2; CGNL1, THBS2, PDGFRA, and KCNE4; CGNL1, THBS2, PDGFRA, and HPPD; CGNL1, APP, S100A10, and TXNRD1; CGNL1, APP, S100A10, and TPM2; CGNL1, APP, S100A10, and KCNE4; CGNL1, APP, S100A10, and PDGFRA; CGNL1, APP, S100A10, and HPPD; CGNL1, APP, TXNRD1, and TPM2; CGNL1, APP, TXNRD1, and KCNE4; CGNL1, APP, TXNRD1, and PDGFRA; CGNL1, APP, TXNRD1, and HPPD; CGNL1, APP, PDGFRA, and TPM2; CGNL1, APP, PDGFRA, and KCNE4; CGNL1, APP, PDGFRA, and HPPD; CGNL1, S100A10, TXNRD1, and TPM2; CGNL1, S100A10, TXNRD1, and KCNE4; CGNL1, S100A10, TXNRD1, and PDGFRA; CGNL1, S100A10, TXNRD1, and HPPD; CGNL1, S100A10, PDGFRA, and TPM2; CGNL1, S100A10, PDGFRA, and KCNE4; CGNL1, S100A10, PDGFRA, and HPPD; CGNL1, TXNRD1, PDGFRA, and TPM2; CGNL1, TXNRD1, PDGFRA, and KCNE4; CGNL1, TXNRD1, PDGFRA, and HPPD; VMO1, THBS2, APP, and S100A10; VMO1, THBS2, APP, and TXNRD1; VMO1, THBS2, APP, and TPM2; VMO1, THBS2, APP, and KCNE4; VMO1, THBS2, APP, and PDGFRA; VMO1, THBS2, APP, and HPPD; VMO1, THBS2, S100A10, and TXNRD1; VMO1, THBS2, S100A10, and TPM2; VMO1, THBS2, S100A10, and KCNE4; VMO1, THBS2, S100A10, and PDGFRA; VMO1, THBS2, S100A10, and HPPD; VMO1, THBS2, TXNRD1, and TPM2; VMO1, THBS2, TXNRD1, and KCNE4; VMO1, THBS2, TXNRD1, and PDGFRA; VMO1, THBS2, TXNRD1, and HPPD; VMO1, THBS2, PDGFRA, and TPM2; VMO1, THBS2, PDGFRA, and KCNE4; VMO1, THBS2, PDGFRA, and HPPD; VMO1, APP, S100A10, and TXNRD1; VMO1, APP, S100A10, and TPM2; VMO1, APP, S100A10, and KCNE4; VMO1, APP, S100A10, and PDGFRA; VMO1, APP, S100A10, and HPPD; VMO1, APP, TXNRD1, and TPM2; VMO1, APP, TXNRD1, and KCNE4; VMO1, APP, TXNRD1, and PDGFRA; VMO1, APP, TXNRD1, and HPPD; VMO1, APP, PDGFRA, and TPM2; VMO1, APP, PDGFRA, and KCNE4; VMO1, APP, PDGFRA, and HPPD; VMO1, S100A10, TXNRD1, and TPM2; VMO1, S100A10, TXNRD1, and KCNE4; VMO1, S100A10, TXNRD1, and PDGFRA; VMO1, S100A10, TXNRD1, and HPPD; VMO1, S100A10, PDGFRA, and TPM2; VMO1, S100A10, PDGFRA, and KCNE4; VMO1, S100A10, PDGFRA, and HPPD; VMO1, TXNRD1, PDGFRA, and TPM2; VMO1, TXNRD1, PDGFRA, and KCNE4; VMO1, TXNRD1, PDGFRA, and HPPD; THBS2, APP, S100A10, and TXNRD1; THBS2, APP, S100A10, and TPM2; THBS2, APP, S100A10, and KCNE4; THBS2, APP, S100A10, and PDGFRA; THBS2, APP, S100A10, and HPPD; THBS2, APP, TXNRD1, and TPM2; THBS2, APP, TXNRD1, and KCNE4; THBS2, APP, TXNRD1, and PDGFRA; THBS2, APP, TXNRD1, and HPPD; THBS2, APP, PDGFRA, and TPM2; THBS2, APP, PDGFRA, and KCNE4; THBS2, APP, PDGFRA, and HPPD; THBS2, S100A10, TXNRD1, and TPM2; THBS2, S100A10, TXNRD1, and KCNE4; THBS2, S100A10, TXNRD1, and PDGFRA; THBS2, S100A10, TXNRD1, and HPPD; THBS2, S100A10, PDGFRA, and TPM2; THBS2, S100A10, PDGFRA, and KCNE4; THBS2, S100A10, PDGFRA, and HPPD; THBS2, TXNRD1, PDGFRA, and TPM2; THBS2, TXNRD1, PDGFRA, and KCNE4; THBS2, TXNRD1, PDGFRA, and HPPD; APP, S100A10, TXNRD1, and TPM2; APP, S100A10, TXNRD1, and KCNE4; APP, S100A10, TXNRD1, and PDGFRA; APP, S100A10, TXNRD1, and HPPD; APP, S100A10, PDGFRA, and TPM2; APP, S100A10, PDGFRA, and KCNE4; APP, S100A10, PDGFRA, and HPPD; APP, TXNRD1, PDGFRA, and TPM2; APP, TXNRD1, PDGFRA, and KCNE4; APP, TXNRD1, PDGFRA, and HPPD; S100A10, TXNRD1, PDGFRA, and TPM2; S100A10, TXNRD1, PDGFRA, and KCNE4; S100A10, TXNRD1, PDGFRA, and HPPD; TXNRD1, TPM2, KCNE4, and PDGFRA; TXNRD1, TPM2, KCNE4, and HPPD; TPM2, KCNE4, PDGFRA, and HPPD.

Preferred combinations of markers include CGNL1 and APP; CD56, CGNL1 and APP; CD166, CGNL1 and APP; CD56, CD166 and CGNL1; and CD56, CD166, CGNL1 and APP; optionally combined with a marker selected from CD56, CD166, CD44 and CD10.

For quality assessment or selection and sorting CECs based on measuring and separation steps as described herein, use may be made of nucleic acid probes or antibodies tethered to nanoparticles. In preferred embodiments use is made of antibodies capable of specifically binding to the marker proteins as indicated herein. Antibodies, as defined herein, against one or more markers selected from CGNL1, VMO1, THBS2, APP, S100A10, TXNRD1, TPM2, KCNE4, PDGFRA, and HPPD, optionally further including CD56, CD166, CD44 and/or CD10 can be obtained from commercial sources such as BD Biosciences, Thermo Fisher Scientific, Abcam, and Prestige Antibodies. Use of antibodies is based on well-known methods of immunolabeling, using either a direct labeling with a single antibody against the marker protein as antigen, or a sandwich approach using a primary antibody and a secondary antibody. A highly preferred combination of antibodies for selecting and sorting therapy-grade cultured CECs includes the use of antibodies against CD56, CD166, CGNL1 and APP.

Although immunostaining approaches regularly include steps of fixing, permeabilization and blocking of cells, it will be understood that in order to provide for native-like CECs that may be useful in therapy, these cells need to remain viable. Hence, immunolabeling in aspects of this invention preferably does not affect the quality of the CECs as they are enriched. Alternatively, it is also possible to sample the culture to assess its quality through a representative subsample by using a destructive technology (e.g., immunofluorescence).

Prior to incubating CECs with one or more antibodies against CGNL1, VMO1, THBS2, APP, S100A10, TXNRD1, TPM2, KCNE4, PDGFRA, and HPPD, optionally further including CD56, CD166, CD44 and/or CD10, non-specific antibody interactions may be blocked, for example by incubating cells with a BSA solution for 1 h at ambient temperature.

CECs may be incubated overnight at 4° C. with a primary antibody against one or more of CGNL1, VMO1, THBS2, APP, S100A10, TXNRD1, TPM2, KCNE4, PDGFRA, and HPPD, optionally further including CD56, CD166, CD44 and/or CD10. The primary antibody against one or more of CGNL1, VMO1, THBS2, APP, S100A10, TXNRD1, TPM2, KCNE4, PDGFRA, and HPPD, optionally further including CD56, CD166, CD44 and/or CD10, may be labeled, with a detectable label, for example a fluorescent label, or incubated with a labeled, secondary antibody. For this, CECs may be washed after primary antibody incubation, for example three times in PBS, and incubated with either fluorescently labeled secondary antibodies for FACS, or with secondary antibodies that are coupled to a carrier material such as beads, preferably magnetic beads, or to monolithic material, preferably monolithic material that is embedded in columns, preferably in micro-columns. The secondary antibody is capable of specifically binding to the primary antibody, e.g. to a detectable label conjugated to the primary antibody or to an epitope part of the primary antibody. Preferably, at least one of the primary or secondary antibody is labeled with a fluorescent label. Suitable fluorescent labels include, for example, Alexa Fluor® dyes (Thermo Fisher Scientific), Dylight® dyes (Dyomics, GmbH, Jena, Germany), Cy3, Cy5 and derivatives thereof such as Cy3.5 (Thermo Fisher Scientific), fluorescein and derivatives such as fluorescein isothiocyanate (FITC, Thermo Fisher Scientific), Pacific dyes such as Pacific Blue™ and Pacific Orange™ (Pacific Industries, Gujarat, India), allophycocyanin (APC; Thermo Fisher Scientific) and rhodamine dyes such as rhodamine 6G, rhodamine 123 and rhodamine B (Rhoda Chem Industries, Gujarat, India).

### Machine Learning Methods

In some embodiments, instead of performing a comparison of the measured level of expression of said at least one gene against a reference level of expression of said at least one gene as measured in a reference sample (reference CEC or reference culture or sample comprising CECs), the quality of cultured CECs may also be determined using a trained machine learning data processing model. For example, reference samples of known quality may be used in order to provide a training set for training a convolutional neural network. After training, the trained machine learning data processing model will then be able to process, as input, the levels of expression of one or more genes in one or more CECs in a sample of cultured CECs of which the quality it to be determined, and provide at the output of the data processing model a determined quality.

In accordance with a further aspect, the disclosure further provides a method of training a machine learning data processing model. The machine learning data processing model may for example include a convolutional neural network, comprising an input and output layer and one or more convolution layers. The convolution layers comprise kernels of which the individual elements (i.e. weights) are to be adapted during training, such as to yield a correct output when at the input a data set is offered.

In the present disclosure, taking the example of a convolutional neural network (CNN) as machine learning data processing model, the training may be performed as follows. Preferably, a supervised learning method is applied, which is characterized by having a desired output for each given set of input signals. In order to train the CNN, a training set is therefore needed of which the correct outcome of the method to be performed is known. Thus, to train the CNN one may start with a reference sample comprising CECs for which the method of the present invention has already been carried out (e.g. manually by comparison and determination) and of which the quality of said one or more CECs in said reference sample is known. The determined outcome, i.e. the data about the quality of the CEC, may be used as ground truth data set. The training data set includes data on the levels of expression of the one or more genes in CECs from the reference sample. The task in training the CNN is to adapt the weights of each kernel of each convolution layer such that eventually, providing the training data set at the input will yield the ground truth data set at the output. If the training has been performed most optimal, comparing the output of the CNN with the ground truth data set will thus yield no or minimal differences for any arbitrary reference sample. Such differences are reflected by the error function, and hence it will be the error function that needs to be minimized eventually during training.

Various adaption strategies are known to the skilled person and may be applied in order to converge the CNN efficiently during training. For example, a gradient descent method may be applied in order to minimize the error function by iteratively adapting the weights of the kernels such as to yield a reduction of the error function with each iteration. The iterations are continued until the reductions become too small or no reduction can be found anymore. Alternative training methods may likewise be applied. This may be done for a CNN with any arbitrary number of layers or kernel sizes. The present disclosure is not limited in terms of numbers of layers or kernel sizes. Furthermore, same can be done with different types of machine learning data processing models, such as a regular artificial neural network consisting of layers with weights and biases for converting neurons, rather than kernels.

Preferably, a large training data set is used in order to train the CNN, however any size of training set will do as long as the results of the trained CNN are satisfactory.

In a method for determining the quality of cultured CECs according to the present invention, step b) of comparing the measured level of expression to a reference level of expression of said at least one gene as measured in a reference CEC or reference sample comprising CECs, may also comprise using a trained machine learning data processing model for automatically associating the level of expression determined in step a) with a quality of the one or more reference CECs, such as to enable providing the quality of the one or more CECs at an output of the machine learning data processing model. In one preferred embodiment of a method of the invention, the level of expression is compared using a machine learning data processing model.

The present disclosure thus provides a method of training a machine learning data processing model for determining the quality of CECs in (a sample of) a culture of CECs, the method comprising the steps of:
a) receiving, by the machine learning data processing model, a training data set comprising training data indicative of a first level of expression of one or more of CGNL1, VMO1, THBS2, APP, S100A10, TXNRD1, TPM2, KCNE4, PDGFRA, and HPPD in a representative number of first CECs within (a sample of) a culture of CECs, wherein preferably the first level of expression is determined using immunological staining techniques optionally in combination with microscopy and image analysis or flow cytometry such as by FACS, or by scRNAseq;
b) receiving, by the machine learning data processing model, a ground truth data set comprising ground truth data indicative of the quality of the first CECs, wherein the quality of the first CECs is determined by comparing the level of expression determined under a) with the level of expression in reference CECs of known quality;
c) training the machine learning data processing model based on the training data received in step a) and the ground truth data received in step b) for enabling, after completion of the training period, the step of automatically associating measurement data indicative of a second level of expression of one or more of CGNL1, VMO1, THBS2, APP, S100A10, TXNRD1, TPM2, KCNE4, PDGFRA, and HPPD in at least one second individual CEC with the quality of the at least one second CEC, such as to enable providing the quality of the at least one second CEC at an output of the machine learning data processing model.

### Computer Implemented Aspects of the Invention

In accordance with the present disclosure, there is also provided a system for determining the quality of CECs in (a sample of) a culture of CECs. Such a system comprises one or more processors for receiving measurement data indicative of a level of expression of one or more of CGNL1, VMO1, THBS2, APP, S100A10, TXNRD1, TPM2, KCNE4, PDGFRA, and HPPD in at least one individual CEC within (a sample of) a culture of CECs. The system further may include a memory storing a trained machine learning model, e.g. such as the trained CNN described above. The trained machine learning model is configured to output a classification of the at least one individual CEC based on the measurement data. The one or more processors are configured to determine data indicative of the quality of the at least one individual CEC based on the output classification. Quality may be expressed as native-like or non-native-like.

The present invention thus provides a system for determining the quality of a CEC in (a sample of) a culture of CECs, the system comprising:
one or more processors for receiving measurement data indicative of a level of expression of one or more of CGNL1, VMO1, THBS2, APP, S100A10, TXNRD1, TPM2, KCNE4, PDGFRA, and HPPD in at least one individual CEC within (a sample of) a culture of CECs; and
a memory storing a trained machine learning model, wherein the trained machine learning model is configured to output a classification of the at least one individual CEC based on the measurement data; and
wherein the one or more processors are configured to determine data indicative of the age of the at least one individual platelet based on the output classification.

In preferred embodiments of method aspects of the present invention, the step of comparing measured gene expression levels with reference gene expression levels is performed on a computer using an algorithm wherein the results of measured gene expression are compared with reference values in CECs of native-like or non-native like quality, preferably wherein said reference values are stored on an accessible memory, and preferably wherein said algorithm can receive the results of measurement of gene expression levels of at least one of CGNL1, VMO1, THBS2, APP, S100A10, TXNRD1, TPM2, KCNE4, PDGFRA, and HPPD of a CEC as described herein.

The present invention thus provides a computer-implemented method for determining the quality of cultured CECs comprising steps of
a) receiving data representing a level of expression of at least one gene selected from CGNL1, VMO1, THBS2, APP, S100A10, TXNRD1, TPM2, KCNE4, PDGFRA, and HPPD measured in or on at least one cultured CEC, and
b) determining the quality of a cultured CEC based on comparing said data received under a) with reference data of the level of expression of said at least one gene as measured in a reference CEC or reference sample of CECs,
wherein step b) is preferably performed on a computer using an algorithm wherein the data received in step a) are compared with reference data (e.g. with reference values of the level of expression of said at least one gene in CECs of native-like quality or non-native like quality), preferably wherein said reference values are stored on an accessible memory, and preferably wherein said algorithm can receive the data of step a).

In another aspect, the present invention provides a computer program product comprising instructions or program portions stored on a computer readable medium for performing the computer-implemented method of the present invention when running on or loaded into a computer, wherein said program portions comprise an algorithm wherein the results of measurement of gene expression levels of at least one of CGNL1, VMO1, THBS2, APP, S100A10, TXNRD1, TPM2, KCNE4, PDGFRA, and HPPD of a CEC are compared with reference values in CECs of native-like or non-native like quality, preferably wherein said reference values are stored on an accessible memory, and preferably wherein said algorithm can receive the results of measurement of gene expression levels of at least one of CGNL1, VMO1, THBS2, APP, S100A10, TXNRD1, TPM2, KCNE4, PDGFRA, and HPPD of a CEC as described herein. In computer program products of the present invention, said instructions or program portions preferably cause the computer to carry out the steps of the computer-implemented method of the present invention when executed by a computer.

The present invention further provides a data processing system comprising a processor adapted to perform the steps of the computer-implemented method of the invention as described above.

The present invention further provides a computer program product comprising instructions which, when the program is executed by a computer, cause the computer to carry out the computer-implemented method of the invention as described above. A computer program product of the present invention may further comprise the machine learning data processing model described above and a ground truth data set as described above.

The present invention further provides a computer-readable medium having stored thereon the computer program product as described above.

The present invention further provides a kit of parts for determining the quality of a cultured CEC, the kit comprising: (a) a binding agent that selectively binds a gene expression product of least one gene selected from CGNL1, VMO1, THBS2, APP, S100A10, TXNRD1, TPM2, KCNE4, PDGFRA, and HPPD, (b) packaging materials and instructions for measuring the level of expression of said at least one gene in a CEC or a sample of CECs, the kit being present in association with instructions for use in any of the methods described herein. Said binding agent may in preferred embodiments comprise an antibody, an aptamer or a complementary nucleic acid sequence. In preferred embodiments, the binding agent comprises forward and reverse primers for an amplification reaction, for example a PCR reaction. The PCR reaction may be any type of PCR reaction, for example a RT-qPCR. The binding agent. Packaging materials and instructions may comprise reference expression level values for native-like and non-native-like CECs as described herein. Kits described herein with reference to CGNL1, VMO1, THBS2, APP, S100A10, TXNRD1, TPM2, KCNE4, PDGFRA, and HPPD may be adapted to detect or measure the expression levels of one or more, such as 2, 3, 4, 5, 6, 7, 8, 9, or 10 of these genes relative to a reference sample.

According to another aspect of the present invention there is provided an array for determining the quality of a cultured CEC, the array comprising a binding agent specific to a gene expression product of least one gene selected from CGNL1, VMO1, THBS2, APP, S100A10, TXNRD1, TPM2, KCNE4, PDGFRA, and HPPD. Preferably an array for determining the quality of a cultured CEC comprises binding agents specific for gene expression products of 2, 3, 4, 5, 6, 7, 8, 9, or 10 of these genes, optionally further comprises binding agents specific for gene expression products of CD56, CD166, CD44 and/or CD10.

According to another aspect of the present invention, there is provided a use of a gene expression product of least one gene selected from CGNL1, VMO1, THBS2, APP, S100A10, TXNRD1, TPM2, KCNE4, PDGFRA, and HPPD as a biomarker for determining the quality of a cultured CEC.

### Enrichment of CECs

CECs may be enriched based on a preferential (native-like or non-native-like) marker phenotype as defined herein by any method for cell sorting known in the art. The use of FACS to separate cells having a first phenotype from cells having another phenotype on the basis of differential staining with antibodies is well known in the art.

If a cell population comprises non-native-like CECs, sorting based on the expression level of at least one marker selected from CGNL1, VMO1, THBS2, APP, S100A10, TXNRD1, TPM2, KCNE4, PDGFRA, and HPPD, optionally further including CD56, CD166, CD44 and/or CD10, may be performed to enrich or select for native-like CECs. For example, a cell population that comprises more than 5% non-native-like CECs, such as more than 10% non-native-like CECs, more than 15% non-native-like CECs, more than 20% non-native-like CECs, may be sorted to enrich or select for native-like CECs.

The secondary antibodies that may for instance be coupled to a carrier may be coupled directly to beads or another monolithic material, or indirectly, for example by coupling of a further antibody that specifically recognizes the marker-specific antibody. Said antibodies preferably are indirectly coupled to the beads or monolithic material by coupling of protein A, protein G, or a mixture of protein A and G to the beads or to the monolithic material. Said antibodies, such as monoclonal or polyclonal antibodies, are preferably coupled to protein A-coupled beads or protein A-coupled monolithic material.

As an alternative, or in addition, native-like CECs may be separated from non-native-like CECs by immunomagnetic sorting of cells on a microfluidic chip (Mair et al., 2019. Nature Biomedical Engineering 3: 796-805). Immunomagnetic sorting of cells on a microfluidic chip may surpass the throughput of traditional FACS-based cell sorting technologies, and may expand the applicability of functional phenotypic screening to fragile cell types such as CECs.

A method for quality assessment or selection and separation based on differentiating between native-like CECs and a non-native-like CECs according to the invention will provide a cell population of CECs that is at least enriched for native-like CECs such as therapy-grade CECs. Said cell population may comprise one or more antibodies against at least one marker selected from CGNL1, VMO1, THBS2, APP, S100A10, TXNRD1, TPM2, KCNE4, PDGFRA, and HPPD, optionally further including antibodies against CD56, CD166, CD44 and/or CD10.

Said cell population of native-like CECs, preferably human CECs, comprises CECs of which at most 10-15%, preferably at most 1-5%, more preferably 0% to at most 1% show increased differential expression of at least one, preferably at least 2, 3, 4, 5, 6, 7 or preferably all of markers VMO1, THBS2, S100A10, TXNRD1, TPM2, KCNE4, PDGFRA, and HPPD.

Said cell population of native-like CECs, preferably human CECs, comprises CECs of which at least 85-90%, preferably at least 90-95%, more preferably at least 95-100% show increased differential expression of at least one, preferably at least 2 of markers CGNL1 and APP.

Said population of native-like CECs, preferably human CECs, comprises CECs of which at most 10-15%, preferably at most 5-10%, more preferably 0% to at most 1% show increased differential expression of at least 3, preferably at least 4 of markers selected from VMO1, THBS2, S100A10, TXNRD1, TPM2, KCNE4, PDGFRA, HPPD, CD44 and CD10.

Said cell population of native-like CECs, preferably human CECs, comprises CECs of which at least 85-90%, preferably at least 90-95%, more preferably at least 95-100% of the cells show increased differential expression of at least CGNL1 or APP3, preferably CGNL1 and APP, preferably CD56, CD166, CGNL1 and APP.

A population of native-like CECs, preferably human CECs, comprising at least 85-90%, preferably at least 90-95%, more preferably at least 95-100% show increased differential expression of CGNL1 or APP3, preferably of both CGNL1 and APP, more preferably of all of CD56, CD166, CGNL1 and APP, and/or comprises CECs of which at most 10-15%, preferably at most 5-10%, more preferably at most 0 to at most 5% show increased differential expression of at least 3, preferably at least 4 markers selected from VMO1, THBS2, S100A10, TXNRD1, TPM2, KCNE4, PDGFRA, HPPD, CD44 and CD10, can be used, for example, in methods of drug development or drug screening or disease modelling. For drug development or drug screening, a confluent layer of native-like CECs may be incubated with and without a candidate therapeutic compound, followed by analysis of the effect of the therapeutic compound on the native-like CECs, for instance by assessing the barrier function and/or pump function of said native-like CECs due to the presence of said compound. A drug that improves said detectable feature such as barrier function and/or pump function may subsequently be tested for improvement of an endothelial cell dysfunction such as Fuchs' endothelial corneal dystrophy, as Fuchs' dystrophy, bullous keratopathy, iridocorneal endothelial (ICE) syndrome, or other inherited or acquired endothelial disorders.

### Methods of Transplanting CECs

Cultured native-like CECs selected or provided by methods of the present invention may be used in therapy. For this the selected or provided cells may be transplanted into the eye of a subject. Transplantation of CECs may comprise the transplantation of CECs in the form of a cell suspension suspended in a suitable medium for transplantation, or in combination with a carrier or support suitable for transplantation into a subjects eye. The invention therefore further provides a tissue engineered graft material comprising a cell population of CECs according to the invention. Said tissue engineered graft material may comprise a synthetic, biological, (bio)mimetic or hybrid material such as in the form of a surface or scaffold with which cells of native-like CECs selected or obtained according to methods of the invention may be combined, for instance onto which the cells may be seeded, for instance so as to become attached in the form of a monolayer. If said material or scaffold is a biomimetic, the composition is preferably similar to the composition of a Descemet's membrane. Said substrate or scaffold preferably comprises collagen such as collagen type I, IV and/or VIII. Said substrate or scaffold may promote CECs proliferation and support human corneal endothelial cell cultures. Suitable scaffolds include decellularized human tissues and biological membranes, as described in Catala et al., 2022 (Catala et al., 2022. Prog Ret Eye Res 87: 100987), which is incorporated herein by reference. Commercially available scaffolds include gelatin, especially pharmaceutical grade gelatin, Atelocollagen (Koken Co Ltd, Tokyo, Japan) and Vitrigel (the Well Bioscience, North Brunswick, NJ, USA). Synthetic scaffolds that are commercially available include silk fibroin, chitosan, polycaprolactone, poly (L-lactic acid-co-ε-caprolactone copolymer, lysophosphatidic acid, poly (ethylene glycol), tropo-elastin, poly(lactic-co-glycolic) acid, gelatin methacrylate and poly D-L-lactic acid. As an alternative, CECs may be cultured while producing their own extracellular matrix. A tissue engineered graft material thus may comprise a three dimensional cell population of CECs according to the invention that are attached to their own extracellular matrix. Said tissue engineered graft comprises a fully biocompatible construct with a decreased risk of adverse reactions upon implantation.

The invention further provides a method for treating a patient suffering from corneal endothelial dysfunction comprising administering to said patient a cell population according to the invention or a tissue engineered graft material comprising a cell population according to the invention as described above.

In transplantation therapies said step of administering may comprise direct injection into the anterior chamber of a patient's eye. Said injection may be performed with or without removing the recipient own endothelial cells and or Descemet's membrane, or part thereof. After the procedure, a patient may be placed in a face-down position for a period of time, for example for about 3 hours, to increase CEC adherence to the posterior part of the cornea. Adherence may further be enhanced by the use of magnetic force using cells laden with ferro-magnetic beads (Moysidis et al., 2015. Nanomed Nanotechnol Biol Med 11: 499-509). The number of CECs that is injected may range between 1×10⁵ and 1×10⁷ CECs and usually is about 1×10⁶ CECs per cornea. This means that 5-10 patients may be treated with native-like CECs according to the invention from a single donor. As an alternative, said step of administering may comprise surgery, preferably an endothelial keratoplasty (EK) technique such as DSEK or DMEK, or variants thereof, by which a tissue engineered graft material comprising a cell population according to the invention is provided to a patient.

Said step of administering may be combined with medication including, for example, a ROCK inhibitor, to improve properties such as adhesion and survival of the injected cells or transplanted material.

Embodiments of the present invention further include a method for determining the quality of cultured corneal endothelial cells (CECs) comprising the step of
measuring in or on a cultured CEC a level of expression of at least one gene selected from Cingulin-like 1 (CGNL1), Vitelline Membrane Outer Layer 1 Homolog (VMO1), Thrombospondin-2 (THBS2), Amyloid Beta Precursor Protein (APP), S100 Calcium Binding Protein A10 (S100A10), Thioredoxin Reductase 1 (TXNRD1), Tropomyosin 2 (TPM2), Potassium Voltage-Gated Channel Subfamily E Regulatory Subunit 4 (KCNE4), Platelet Derived Growth Factor Receptor Alpha (PDGFRA), and 4-Hydroxyphenylpyruvate Dioxygenase (HPPD), and
comparing said measured level of expression to a reference level of expression of said at least one gene. Such a reference level of expression may for instance be a set value for the reference level of expression, e.g. as previously obtained from a reference cell or sample, preferably a reference CEC or reference sample comprising CECs, or may be measured in a reference CEC or reference sample comprising CECs. The term “reference level of expression as measured in a reference CEC or reference sample of CECs”, as used herein, includes reference to a set value for the reference level of expression of said gene, e.g. as previously obtained from a reference cell or sample, preferably a reference CEC or reference sample comprising CECs, or may be measured in a reference CEC or reference sample comprising CECs. The reference level of expression therefore includes any quantitative reference with which the measured level of expression can be compared in order to determine if a differential expression of said gene is present in the CEC.

In preferred embodiments of a method for determining the quality of cultured CECs, said cultured CEC is a cultured primary CEC, a transdifferentiated endothelial precursor cell, including but not limited to a an induced bone marrow-derived endothelial progenitor cell (BEPC); or a stem-cell-derived CEC, including but not limited to an induced pluripotent stem cell (iPSC), an embryonic stem cell (ESC), a neural crest (NC) stem cell, a mesenchymal stem cell (MSC) and a dental pulp stem cell (DPSC).

In another preferred embodiment of a method for determining the quality of cultured CECs, said reference cell or reference sample is an isolated primary CEC, a corneal endothelium, a cornea, a culture of CECs or a sample or a cell thereof. Preferably, said reference cell or reference sample exhibits increased differential expression of one or more functional CEC markers selected from SLC4A11, ATP1A1, CD166, CD56, PRDX6, PITX2, CDH2 and COL4A3 when compared to a cultured CEC transitioning towards a senescent or fibrotic phenotype. A cultured CEC transitioning towards a senescent or fibrotic phenotype typically does not differentially express these functional CEC markers, but, instead, expresses senescence or fibrotic markers, such as senescence markers CDKN2A and TAGLN, and fibrotic markers CD44 and ACTA2, and the marker CD10, which expression is typically increased in the senescent and fibrotic clusters that denote CECs of low quality.

In another preferred embodiment of a method for determining the quality of cultured CECs, said method further comprises the step of concluding that the cultured CEC represents a native-like CEC when the level of expression of said at least one gene corresponds to the reference level of expression.

In another preferred embodiment of a method for determining the quality of cultured CECs, said step of determining said level of expression is performed at the end of a cultivation period, e.g. just prior to administering the culture of CECs to a patient.

In yet another preferred embodiment of a method for determining the quality of cultured CECs, said step of determining said level of expression is performed at least twice during a period of cultivation of said cultured CEC, e.g. in order to assess the progression of cultivation and quality of the culture during expansion.

In another preferred embodiment of a method for determining the quality of cultured CECs, said method further comprises the step of measuring in said cultured CEC a level of expression of at least one of CD56, CD166, CD44 and CD10.

In yet another preferred embodiment of a method for determining the quality of cultured CECs, said CEC is a human CEC (hCEC).

In preferred embodiments of a method for determining the quality of cultured CECs, said CEC is an individual CEC.

In further preferred embodiments of a method for determining the quality of cultured CECs according to the invention, the level of expression is determined in or on a single cell. Alternatively, the level of expression may be determined in or on a (sub-)population of CECs.

In methods and aspects of the present invention, it is preferred that the level of expression is measured by immunofluorescence techniques, preferably using fluorescently labeled antibodies against (preferably cell surface) proteins expressed by the indicated genes.

In preferred embodiments of a method for determining the quality of cultured CECs according to the invention, the cultured CEC represents a native-like CEC when the level of expression of said at least one gene, i.e. as an expression profile of the transcriptomic or proteomic expression markers as defined herein, corresponds to a CGNL1+, VMO1−, THBS2−, APP+, S100A10−, TXNRD1−, TPM2−, KCNE4−, PDGFRA−, and HPPD− phenotype, optionally wherein the reference expression profile further corresponds to a CD56+, CD166+, CD44− and CD10− phenotype.

In another aspect, the present invention provides a method of selecting a culture of CECs for medical use, the method comprising the steps of:
a) providing an in vitro culture of CECs, preferably cultured primary CECs or stem-cell-derived CECs, more preferably hCECs,
b) performing the method for determining the quality of cultured CECs according to the invention as described above on at least a part of said culture, and
c) selecting said culture of CECs as a culture of CECs for medical use based on the result of said method.

In preferred embodiments of a method of selecting a culture of CECs for medical use, said method further comprises the step of providing the culture thus selected as a culture of CECs for medical use. The step of providing the culture thus selected as a culture of CECs for medical use may comprise:
harvesting the in vitro culture of CECs as culture of CECs for medical use when the incidence of native-like CECs in said culture is at a desired level; or
separating native-like CECs from said in vitro culture of CECs and collecting the thus separated cells as a culture of CECs for medical use; or
removing non-native-like cells from said in vitro culture of CECs and collecting the remainder of said culture as a culture of CECs for medical use.

In preferred embodiments of a method of selecting or providing a culture of CECs for medical use in accordance with the invention, said step a) may comprise cultivating said CECs as a monolayer, optionally dissociating said monolayer, for instance into individual cells or cell clumps, and providing CECs in said optionally dissociated monolayer with fresh tissue culture reagent to produce a subsequent passage.

In preferred embodiments of a method of selecting or providing a culture of CECs for medical use, said cultivation occurs in the presence of a ROCK Kinase inhibitor and/or a p53 inhibitor.

In preferred embodiments of a method of selecting or providing a culture of CECs for medical use, said cells are cultivated in or on a substrate or scaffold.

In another aspect, the present invention provides a culture of CECs, preferably in the form of a monolayer, wherein the cell population in said culture of CECs comprises CECs of which at most 10-15%, preferably at most 1-10% show increased differential expression of at least one, preferably at least 2, preferably 3, 4, 5, 6, 7, preferably all of markers VMO1, THBS2, S100A10, TXNRD1, TPM2, KCNE4, PDGFRA, and HPPD, and of which at least 85-90%, preferably at least 90-99% show increased differential expression of, at least one, preferably at least 2 genes selected from CGNL1 and APP.

In one embodiment, a culture of CECs in accordance with the present invention is preferably selected or provided by the method of selecting or providing a culture of CECs for medical use in accordance with the invention.

In other embodiments, a culture of CECs in accordance with the present invention is preferably selected or provided by the method of selecting or providing a culture of CECs for use in drug development or drug screening or disease modelling in accordance with the invention.

In preferred embodiments of a culture of CECs in accordance with the present invention, said culture comprises antibodies against a protein expression product of at least one gene selected from CGNL1, VMO1, THBS2, APP, S100A10, TXNRD1, TPM2, KCNE4, PDGFRA, and HPPD.

In preferred embodiments of a culture of CECs in accordance with the present invention, the cell population in said culture is about 10-15% positive for (show increased differential expression of) markers CD44 and CD10, and at least 1, preferably at least 2 of VMO, THBS2⁻, S100A10, TXNRD1, TPM2, KCNE4, PDGFRA, and HPPD, and 85-90% positive for (show increased differential expression of) markers CD56, CD166 and at least 1, preferably at least 2 of CGNL1 and APP.

In preferred embodiments of a culture of CECs in accordance with the present invention, said cell population is in the form of a monolayer.

In another aspect, the present invention provides a tissue engineered graft material comprising a culture of CECs in accordance with the present invention.

In yet another aspect, the present invention provides a method for treating a patient suffering from corneal endothelial dysfunction comprising administering to said patient the culture of CECs in accordance with the present invention, or the tissue engineered graft material in accordance with the present invention. Preferably, said treatment is to improve the function of the cornea in said subject. Hence, the form of the culture of CECs or the amount of CECs administered through said culture is aimed to have curative effect on corneal endothelial dysfunction, to ameliorate dysfunction of said cornea, or to restore the corneal endothelial function in said subject.

In preferred aspects of this invention, the methods are in vitro, non-invasive, methods.

In embodiments of aspects of this invention, the level of expression may be measured in or on a single cell, or in or on a multitude of cells, for instance a subsample or aliquot from a CEC culture.

In yet another aspect, the present invention provides a computer-implemented method for determining the quality of cultured CECs comprising steps of:
a) receiving data representing a level of expression of at least one gene selected from CGNL1, VMO1, THBS2, APP, S100A10, TXNRD1, TPM2, KCNE4, PDGFRA, and HPPD measured in or on at least one cultured CEC, and
b) determining the quality of a cultured CEC based on comparing said data received under a) with reference data of the level of expression of said at least one gene as measured in a reference CEC or reference sample of CECs,
wherein step b) is preferably performed on a computer using an algorithm wherein the data received in step a) are compared with reference data, preferably wherein said reference values are stored on an accessible memory, and preferably wherein said algorithm can receive the data of step a).

In yet another aspect, the present invention provides a data processing system comprising a processor adapted to perform the steps of the computer-implemented method of the present invention.

In yet another aspect, the present invention provides a computer program product comprising instructions which, when the program is executed by a computer, cause the computer to carry out the computer-implemented method of the present invention, optionally comprising a machine learning data processing model.

All embodiments described for different aspects of the present invention may be combined and such combinations of disclosed embodiments are themselves an embodiment of aspects of the present invention. All patents and literature references cited in the present specification are hereby incorporated by reference in their entirety. The invention is further explained in the following examples. These examples do not limit the scope of the invention, but merely serve to clarify the invention.

### Examples

### Example 1. A Single-Cell RNA-Seq Analysis Unravels the Heterogeneity of Primary Cultured Human Corneal Endothelial Cells

### Experimental Model and Subject Details

### Research-Grade Donor Human Corneas and Ethical Statement

This study was performed in compliance with the tenets of the Declaration of Helsinki. All research-grade human donor corneas used for primary culture were obtained from the Lions Eye Institute for Transplant & Research (Tampa, USA), with informed consent from the next of kin. The research involving human-derived corneas was performed in accordance to Maastricht University and Dutch national regulations. All corneas had an endothelial cell density of at least 2800 cells/mm², deemed unsuitable for transplantation, and were preserved in Optisol-GS at 4° C. for up to 14 days prior to their use (Table 1).

TABLE 1
Donor cornea information. COD = cause of death, GI =
Gastrointestinal, MVA = motor vehicle accident, ECD =
endothelial cell density, OS = oculus sinister, OD = oculus dexter.

| | | Age | Preservation | | ECD OS/OD |
|---|---|---|---|---|---|
| Donor | Sex | (yrs) | time (days) | COD | (cells/mm²) |
| Donor 1 | Male | 34 | 12 | Trauma | 3195/2917 |
| Donor 2 | Male | 29 | 11 | GI bleed | 2832/2878 |
| Donor 3 | Female | 27 | 14 | MVA | 2861/3159 |

### Method Details

### Isolation and Culture of Primary Human Corneal Endothelial Cells

Six paired corneas, from two male and one female donor aged 27 to 34 years, were used for isolation and primary culture of endothelial cells. Donors had no history of ocular disease, chronic systemic disease, or pathological infection such as HIV, Hepatitis B and C, HTLV-I/II, syphilis, or SARS-CoV-2.

Prior to isolation, the endothelial-Descemet's corneal layer was manually stripped as follows: the corneas were vacuum fixed in a punch base (E.Janach® srl, Como, Italy) endothelial cell side up and trephined with a 10 mm Ø corneal punch at a fixed depth of 100 μm (E.Janach® srl). To delimit the endothelial trephined line, corneas were stained with a trypan blue solution (0.4%) for 30 s, and washed with balanced salt solution sterile irrigating solution (BSS; Alcon). The corneal endothelium was then gently lifted using a DMEK cleavage hook (E.Janach® srl) and fully stripped using angled McPherson tying forceps.

Human corneal endothelial cells were isolated and cultured as previously reported (Peh et al., 2015. Cell Transpl 24: 287-304). Briefly, the stripped endothelium-Descemet's layer was incubated with 2 mg/ml collagenase A (Roche) solution in human endothelial serum free media (SFM) (Thermo Fisher Scientific) for 2-5 h at 37° C. followed by a 5 min incubation in TrypLE express (Thermo Fisher Scientific) to generate small clumps of corneal endothelial cells. Cells from each cornea were seeded equally across 2 wells of a 24-well plate coated with fibronectin collagen (FNC) coating mix (Athena Enzyme Systems) in M5 stabilization media (human endothelial SFM (Thermo Fisher Scientific) supplemented with 5% fetal bovine serum (FBS), 100 U/mL penicillin-streptomycin, and 0.25 μg/mL amphotericin B) supplemented with 10 μM Y-27632 (STEMCELL Technologies, Cambridge, UK). Subsequently, corneal endothelial cells were cultured in M4 proliferation medium (1:1 Ham's F12 (Thermo Fisher Scientific) and M199 (Thermo Fisher Scientific) supplemented with 5% FBS, 20 μg/mL ascorbic acid (Sigma), 1×ITS (Thermo Fisher Scientific), 10 ng/mL human recombinant bFGF (Sigma), and 10 μM Y-27632 (STEMCELL Technologies)); media was refreshed every other day. Upon reaching 90% confluency, after approximately 8-10 days of culture, cells were cultured in M5 stabilization media for 7 days. After this, corneal endothelial cells were treated with TrypLE express and passaged into wells pre-coated with FNC-coating mix at a seeding density of 10,500 cells/cm² in M5 stabilization medium. All cell culture was performed in incubators with humidified atmosphere of 37° C. and 5% CO₂.

### Preparation of Single Cell Suspension and Methanol Fixation

Cells from five different culture time points were methanol fixed for sequencing. Namely, cells at days 2 and 5 of culture after isolation in M4 proliferation media at passage 0, and cells at confluency after 7 days of culture in M5 stabilization media, at passages 0, 1, and 2. To generate a single cell suspension, primary cultured corneal endothelial cells were treated with TrypLE express for approximately 30 min at 37° C. Then cells were centrifuged for 5 min at 800×g and resuspended in 1 mL ice-cold Dulbecco's phosphate-buffered saline (DPBS, Thermo Fisher Scientific). Next, the cells were centrifuged for 5 min at 800×g and resuspended in ice-cold DPBS at a ratio of 200 μL DPBS/1×10⁶ cells, followed by the dropwise addition of ice-cold methanol at a ratio of 800 μL DPBS/1×10⁶ cells. The fixed cell suspensions were stored at −80° C. until sequencing.

### Single-Cell RNA Sequencing

scRNAseq of primary cultured CECs was performed at Single Cell Discoveries (Utrecht, the Netherlands) following standard 10×Genomics 3′ V3.1 chemistry protocol (10×Genomics, Chromium Next GEM Single Cell 3′ Reagent Kits v3.1 User Guide Rev D, Doc CG000204, November 2019). Cells were rehydrated and loaded on the 10×Chromium controller as follows. Approximately 10,000 cells were loaded per each sample specified in Table 2. The resulting sequencing libraries were prepared following the standard 10×Genomics protocol and sequenced with an Illumina NovaSeq 6000 platform; read length: 150 bp, paired-end.

TABLE 2
10x genomics sample loading and library information

| Library | Donor and time point (cell count ratio) |
|---|---|
| G1 | Donor 3 day 5 proliferation + donor 2 |
| | confluency passage 0 (1:3) |
| G2 | Donor 3 confluency passage 0 + donor 2 |
| | day 5 proliferation (3:1) |
| G3 | Donor 3 confluency passage 1 + donor 1 |
| | day 2 proliferation (3:1) |
| G4 | Donor 3 confluency passage 2 + donor 2 |
| | day 2 proliferation (3:1) |
| G5 | Donor 1 confluency passage 0 |
| G6 | Donor 1 confluency passage 1 |
| G7 | Donor 1 confluency passage 2 |
| G8 | Donor 2 confluency passage 1 |
| G9 | Donor 2 confluency passage 2 |

Bioinformatic Analysis of scRNA-Seq Data

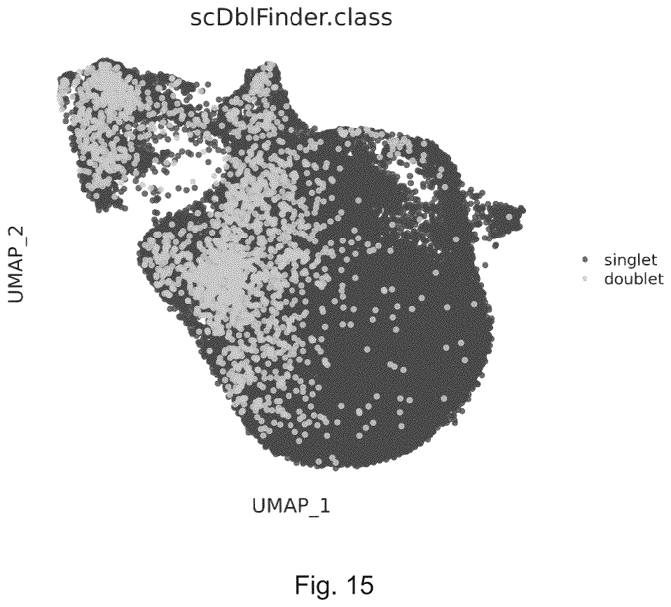

The BCL files resulting from sequencing were transformed to FASTQ files with 10×Genomics Cell Ranger mkfastq following its mapping with Cell Ranger count. During sequencing, Read 1 was assigned 28 bp, and were used for identification of the Illumina library barcode, cell barcode and unique molecular identifier (UMI). Read 2 was used to map the human reference genome GRCh38 (Genbank: GCA_000001405.29). Filtering of empty barcodes was done in Cell Ranger. The data from all samples were loaded in R (version 4.2.0) (R Core Team, 2013. R: A language and environment for statistical computing. R Foundation for Statistical Computing, Vienna, Austria. URL: R-project.org) and processed using the Seurat package (version 4.1.1) (Stuart et al., 2019. Cell 177: 1888-1902). More specifically, for each library a UMI cutoff was used to filter out low quality cells because of the differences between the libraries (i.e. g1—500, g2—3000, g3—500, g4—1313, g5—4000, g6—4000, g7—4000, g8—4000, g9—4000) (Table 2). Additionally, cells with less than 10% mitochondrial gene content were retained for analysis. The data of all 10× libraries were merged and processed together. The merged dataset was normalized for sequencing depth per cell and log-transformed using a scaling factor of 10,000. The multiplexed samples were demultiplexed based on their single nucleotide polymorphism (SNP) profile using Souporcell v2.0 (Heaton et al., 2020. Nat Methods 17: 615-620; URL: github.com/wheaton5/souporcell). Briefly, the Bam file and barcodes of each library were used as input together with the reference genome GRCh38. Besides the default parameters, the number of clusters was set to the number of multiplexed samples per library. The demultiplexing information for each cell was added to the metadata object in Seurat. The patient and library effect was corrected using Harmony (Korsunsky et al., 2019. Nat Methods 16: 1289-1296), as implemented in Seurat and used for dimensionality reduction and clustering of all cells. Cells were clustered using graph-based clustering and the original Louvain algorithm (Blondel et al., 2008. J Stat Mech: P10008) was utilized for modularity optimization. The differentially expressed genes per cluster were calculated using the Wilcoxon rank sum test and used to identify cell types. Putative doublets were computationally identified using scDblFinder (version 1.2.0) (Germain, 2020, URL: github.com/plger/scDblFinder) but did not compose a separate cluster and therefore were not removed from the dataset (FIG. 15). Pseudo time analysis was performed using the Monocle-3 package (version 1.0.0) (Cao et al., 2019. Nature 566, 496-502). Gene set enrichment analysis was performed on lists of differentially regulated genes without prefiltering step. Gene lists were preranked using the signed −log 10 (P adj) and subjected to enrichment analysis using fgsea package (version 1.22.0) (Korotkevich et al., 2021. BioRxiv: doi.org/10.1101/060012) and gage (version 2.46.1) (Luo et al., 2009. BMC Bioinformatics 10: 161) with curated and hallmark gene sets from MSigDB Collections (version 7.5.1) (Liberzon et al., 2015. Cell Syst 1: 417-425; Subramanian et al., 2005. PNAS 102: 15545-15550). To prune selectively the resulting pathways and GO terms, enrichment was considered when up- or downregulated gene sets were detected using both methods. The CEC dataset was integrated with the previously published cornea atlas (Catala et al., 2021. Sci Rep 11: 21727). The library effect was corrected for using harmony, followed by dimensionality reduction. The cluster information from the separate analysis was used to overlay in 2D space. The cell type of the atlas cells was predicted using the CEC confluency dataset using the cell label transfer functionality from Seurat.

### Immunofluorescence

Primary cultured CECs at time point confluency passage 2 deriving from all three donors were used for immunofluorescence analysis. CECs were fixed in 4% PFA for 15 min at ambient temperature and the cells were permeabilized with 0.1% (v/v) Triton X-100 in phosphate buffered saline (PBS) for 10 min. After permeabilization, non-specific antibody interactions were blocked with blocking buffer (2% (w/v) BSA solution in PBS) for 1 h at ambient temperature. CECs were incubated overnight at 4° C. with primary antibodies mouse monoclonal anti-CD166 [3A6](1:200 dilution, BD Biosciences), rabbit polyclonal anti-ZO1 (1:100 dilution, Thermo Fisher Scientific), mouse monoclonal anti-CD44 [Hermes-3](1:400 dilution, Abcam), rabbit polyclonal anti-VMO1 (1:100 dilution, Prestige Antibodies), rabbit polyclonal anti-THBS2 (1:100 dilution, Abcam), rabbit monoclonal anti-CD10 [EPR22867-118](1:100 dilution, Abcam), rabbit monoclonal anti-NCAM1 [CAL53](1:100 dilution, Abcam), and rabbit polyclonal anti-CGNL1 (1:200 dilution, Atlas antibodies) diluted in blocking buffer. After primary antibody incubation, tissues were washed three times in PBS and then incubated with secondary antibodies goat anti-mouse A488 (1:400 dilution; Thermo Fisher Scientific), and donkey anti-rabbit A568 (1:400 dilution; Thermo Fisher Scientific) diluted in blocking buffer for 50 min at ambient temperature in the dark. Cell nuclei were stained with 1 μg/mL Hoechst 33342 (Thermo Fisher Scientific) for 10 min. The CEC samples were then washed three times in PBS and examined on an Eclipse Ti-E inverted microscope (Nikon) equipped with an X-Light V2-TP spinning disk (Crest Optics).

Statistical and Quantitative Analysis of scRNAseq Data

All the statistical analysis for scRNAseq were performed in R (version 4.2.0) with the packages described in the methods detail section. Briefly, differentially expressed genes were detected using a Wilcoxon Rank-Sum test, statistical significance was defined as p<0.01. GSEA revealed differentially enriched gene sets from MSigDB Collections, statistical significance was defined as p<0.05.

### Results

scRNAseq Reveals Different Subpopulations of CECs Arising from Primary Culture

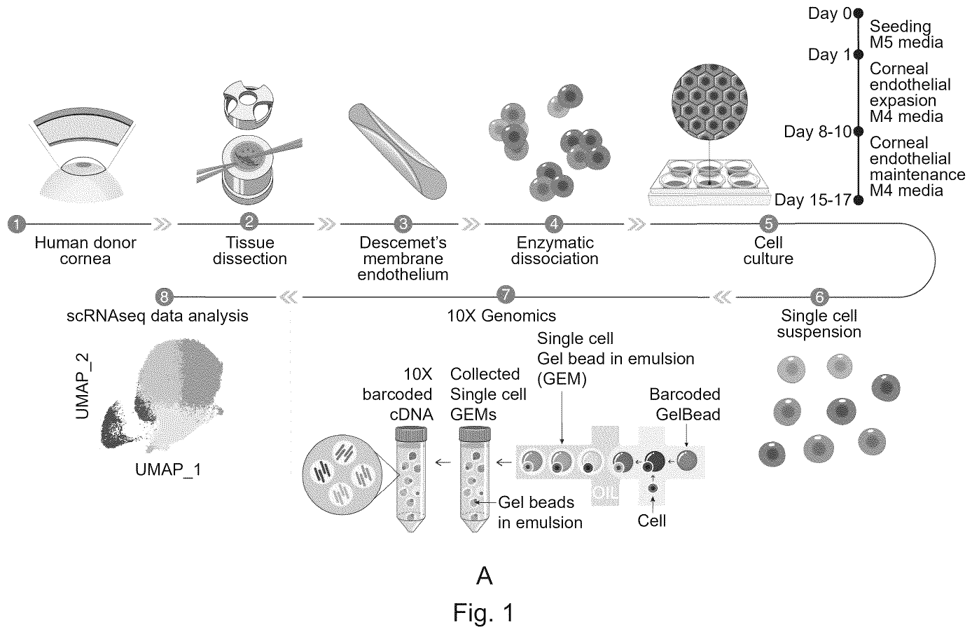

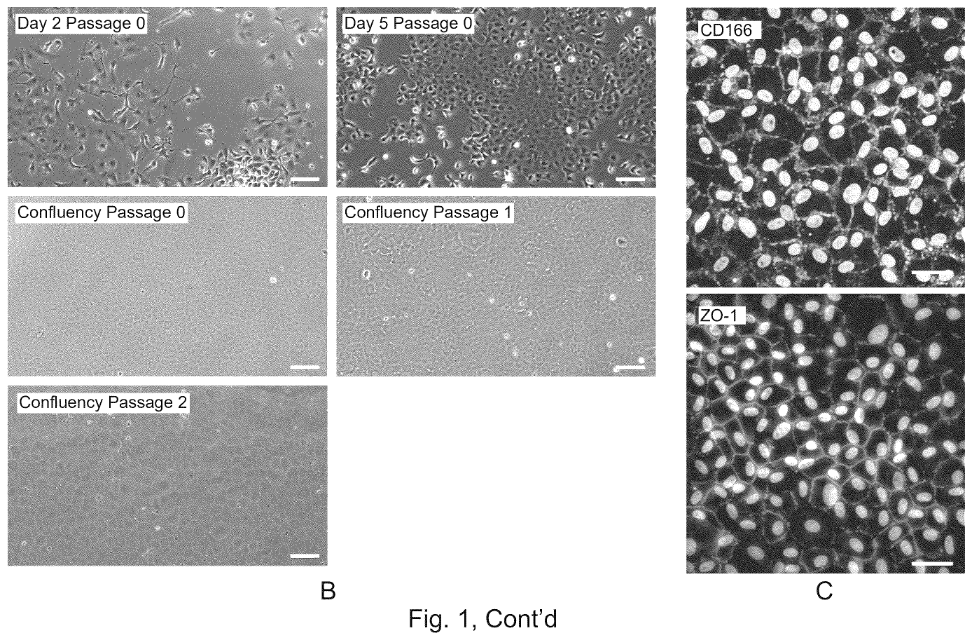

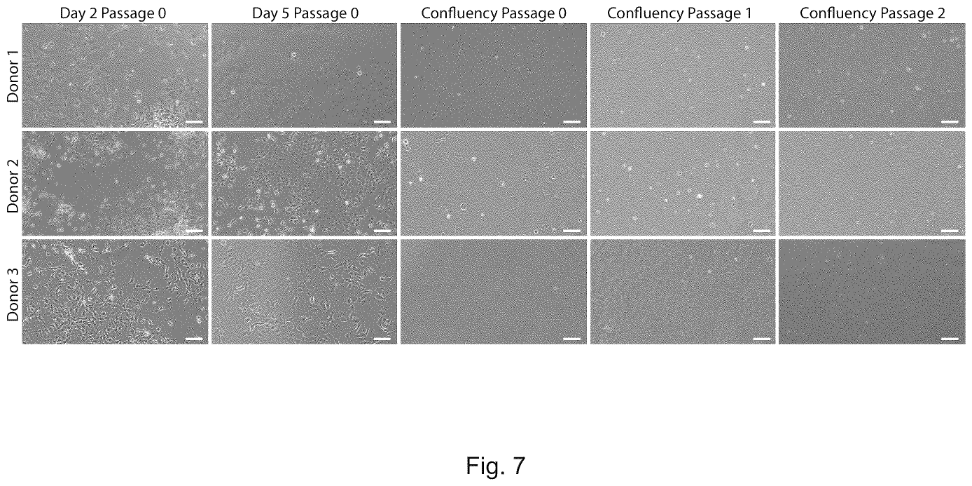

Six paired human corneas, from two male and one female donor (Table 1), were used for isolation, primary culture, and scRNAseq of CECs (FIG. 1A). Cells from five different time points in culture were loaded for sequencing as specified in Table 2. Namely, cells at days 2 and 5 of culture after their isolation in M4 proliferation media at passage 0, and cells at passages 0, 1, and 2, at confluency after 7 days of culture in M5 stabilization media. The cultures showed characteristic CEC morphology over time (FIG. 1B, FIG. 7) and expressed the desired CEC proteins such as CD166 and zonula occludens-1 (ZO-1) (FIG. 1C).

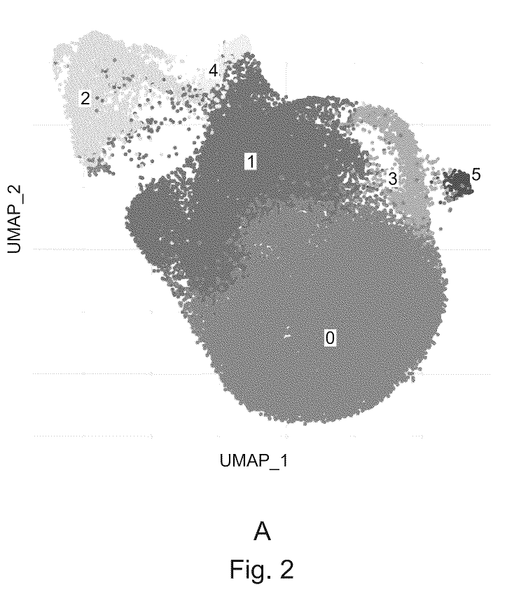

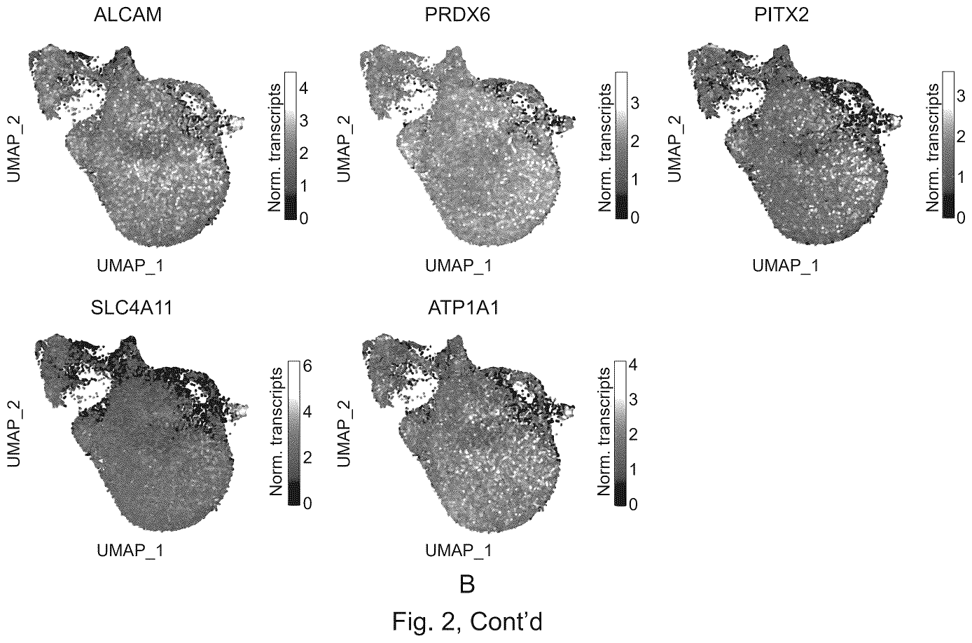

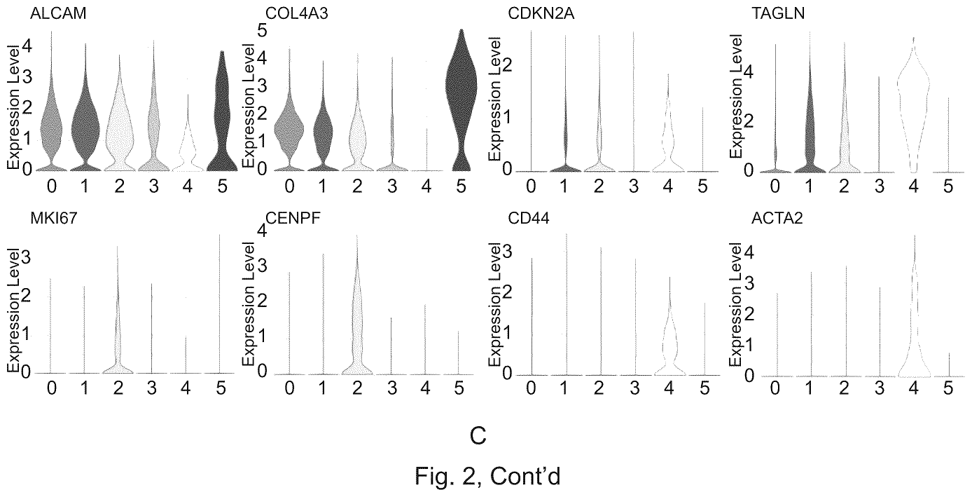

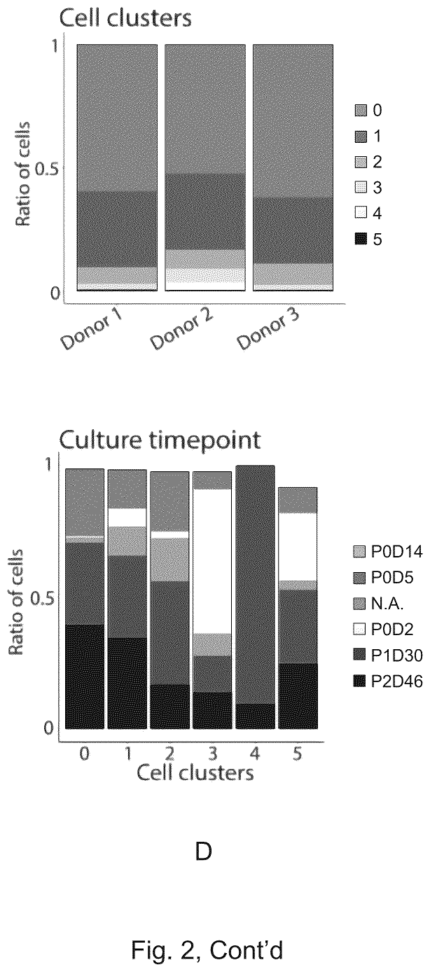

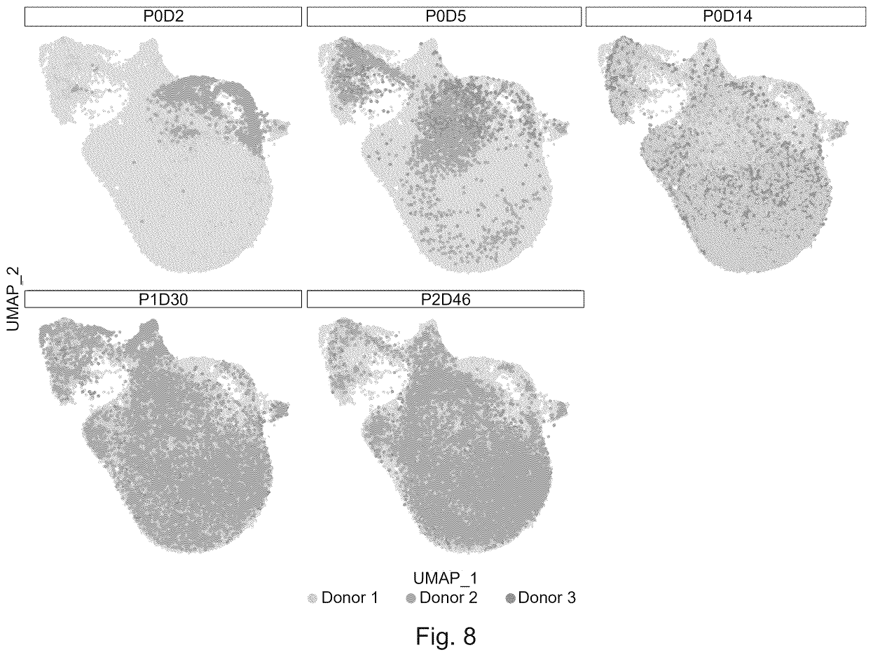

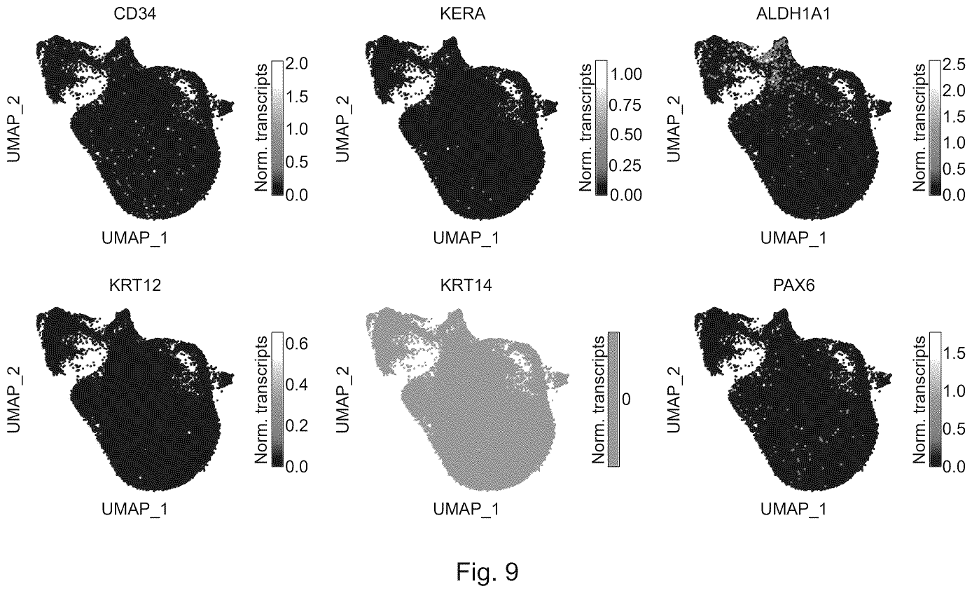

After filtering for cells with a minimum of 1,000 transcripts, the transcriptome profiles of 42,220 cells were embedded in a uniform manifold approximation and projection (UMAP). Cells from all donors were distributed homogeneously across the UMAP (FIG. 8). Unbiased low-resolution clustering revealed six major cell clusters (FIG. 2A), all of which expressed CEC markers ALCAM (CD166), PRDX6, SLC4A11, PITX2, and ATP1A1 (Van den Bogerd et al., 2019. Transl Vis Sci Technol 8: 13; Catali et al., 2020. Cornea 39: 1407-1414; Catali et al., 2022. Prog Retin Eye Res 87: 100987), confirming their endothelial identity (FIG. 2B). The absence of keratocyte markers CD34, KERA, and ALDH1A1, (Férnandez-Pérez and Ahearne, 2019. Curr Eye Res 44: 135-146; Foster et al., 2015. Sci Rep 5: 10839) and epithelial markers KRT12, KRT14, and PAX6 (Hayashi et al., 2016. Nature 531: 376-380; Ouyang et al., Nature 511: 358-361) confirmed the absence of contaminating cell types from corneal stroma and epithelium (FIG. 9). Differential gene expression profiling was used for cluster identification.

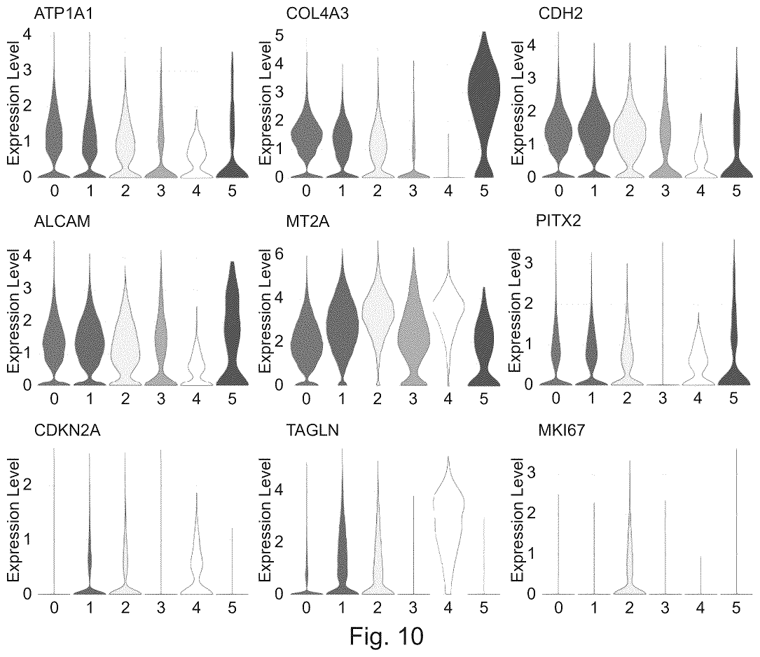

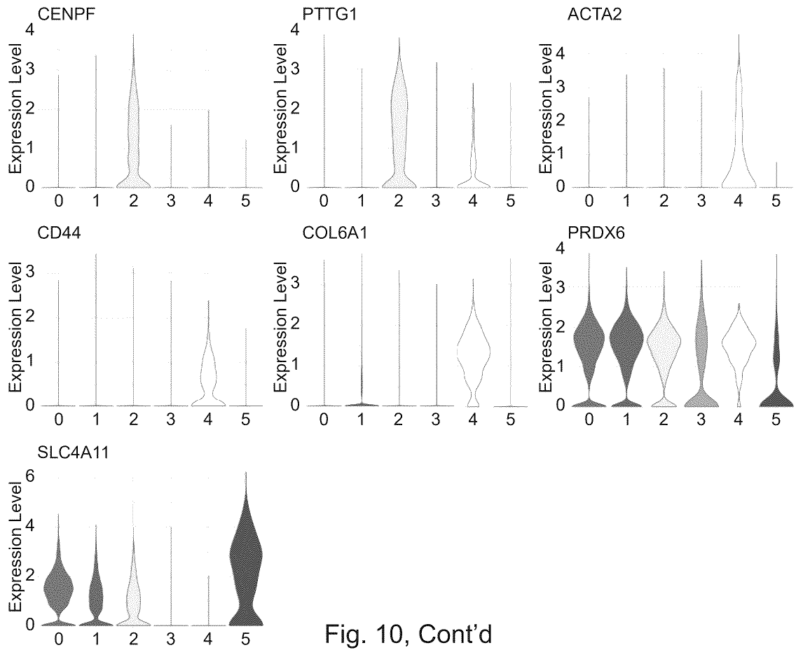

Clusters 0 and 5 presented increased differential gene expression of typical CEC markers, SLC4A11, COL4A3 (Van den Bogerd et al., 2019. Transl Vis Sci Technol 8: 13), CDH2 (He et al., 2016. Sci Rep 6: 29047), and ALCAM, compared to the other sequenced cells, suggesting these clusters were composed of therapy-grade CECs (FIGS. 2C and 10). Cluster 1 was identified as CECs transitioning towards a senescent phenotype due to the high differential expression of senescence markers such as MT2A (Malavolta et al., 2017. Exp. Gerontol. 99: 35-45), CDKN2A (p16) (Enomoto et al., 2006. Vis Sci 47: 4330-4340), and TAGLN (Benadjaoud et al., 2022. Int J. Radiat Oncol Biol Phys 112: 975-985) (FIGS. 2C and 10). Cluster 2 was composed of highly proliferative CECs expressing MKI67 (Sun and Kaufman, 2018, Chromosoma 127: 175-186), CENPF (Varis et al., 2006. Chromosoma 115: 288-295), and PTTG1 (Ersvwr et al., 2020. Mod Pathol 33: 905-915) (FIG. 2C). Cluster 4 was composed of fibrotic CECs with increased differential expression of ACTA2 (α-smooth muscle actin (SMA)) (Rao et al., 2014. J Clin Diagnostic Res 8: 14-17), CD44 (Hamuro et al., 2016. Vis Sci 57: 4452-4463), and COL6A1 (Oouchi et al., 2021. Int J Mol Sci 22: 7335) (FIGS. 2C and 10). Finally, our analysis revealed that cluster 3 was mainly composed of cells in passage 0 (at both day 2 and day 5) (FIG. 2D) that differentially expressed ribosomal-associated genes (FIG. 11). This finding suggested that cells at an early culture stage clustered together due to the necessary adaptation to in vitro culture conditions and the use of proliferation media, which led us to further explore the cells that had been cultured to confluency in passages 0, 1 and 2.

scRNAseq Reveals Seven Distinct Subpopulations of Primary Cultured CECs at Therapeutically Relevant Time Points

To identify the meaningful differences at therapeutically relevant time points and remove the clustering bias introduced by the adaptation to primary culture conditions after cell isolation, we separately analyzed the CECs at confluency in passages 0, 1, and 2. These time points are the most therapeutically relevant, as CECs are most suitable for therapy after 7 days in M5 stabilization media up to passage 2 (Frausto et al., 2020. Sci Rep 10: 7402; Peh et al., 2019. Sci Rep 9: 6087).

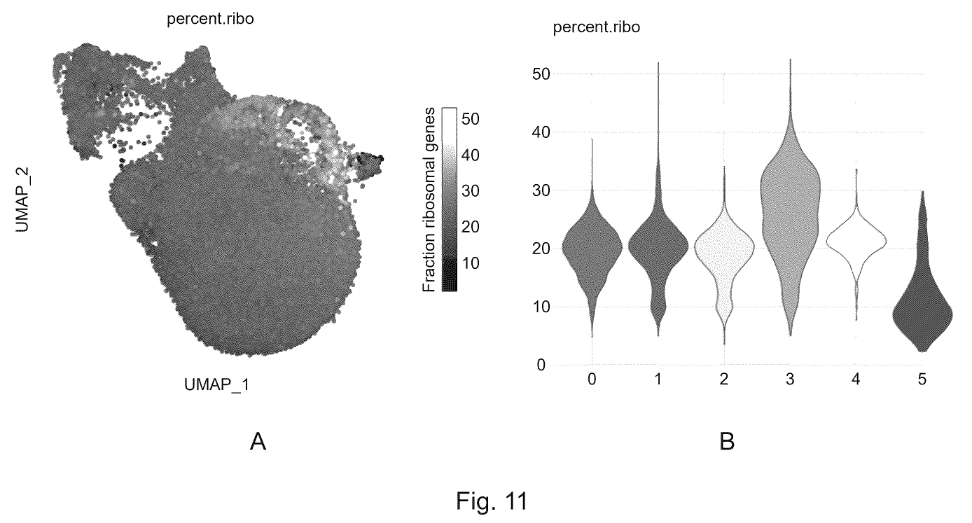

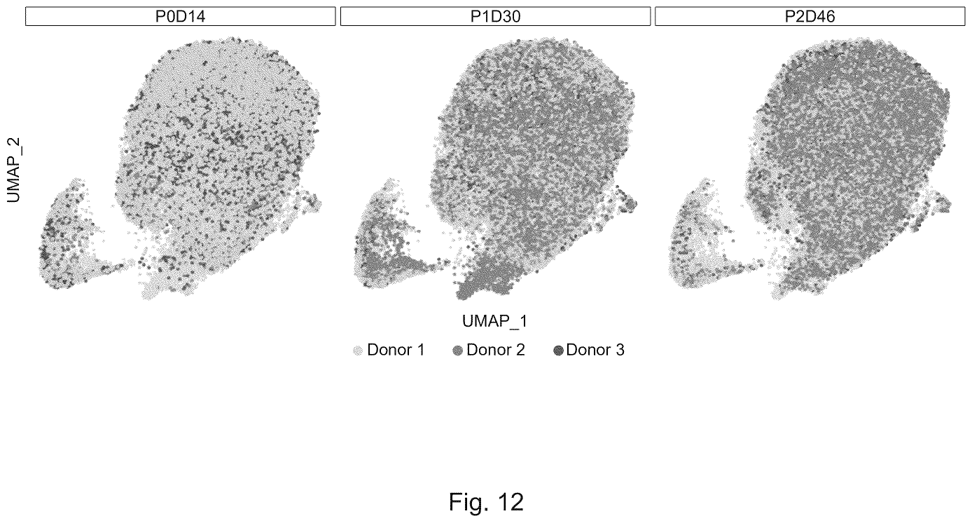

After removing sub-confluent cells from day 2 and day 5 (in passage 0) of culture, the transcriptome profiles of 37,158 CECs at confluency in passages 0, 1, and 2 were embedded in a UMAP. Unbiased low-resolution clustering revealed seven cell clusters (FIG. 3A) with distinct transcriptomic signatures. Cells from all donors were distributed homogeneously across the UMAP (FIG. 12). Differential gene expression analysis was used for identification of each cell cluster (C0-C6).

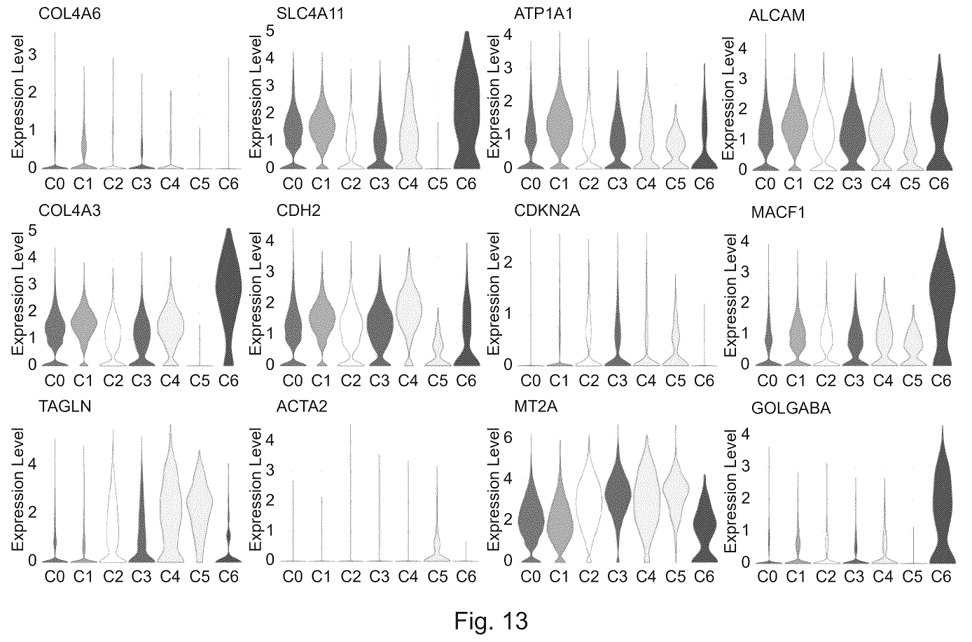

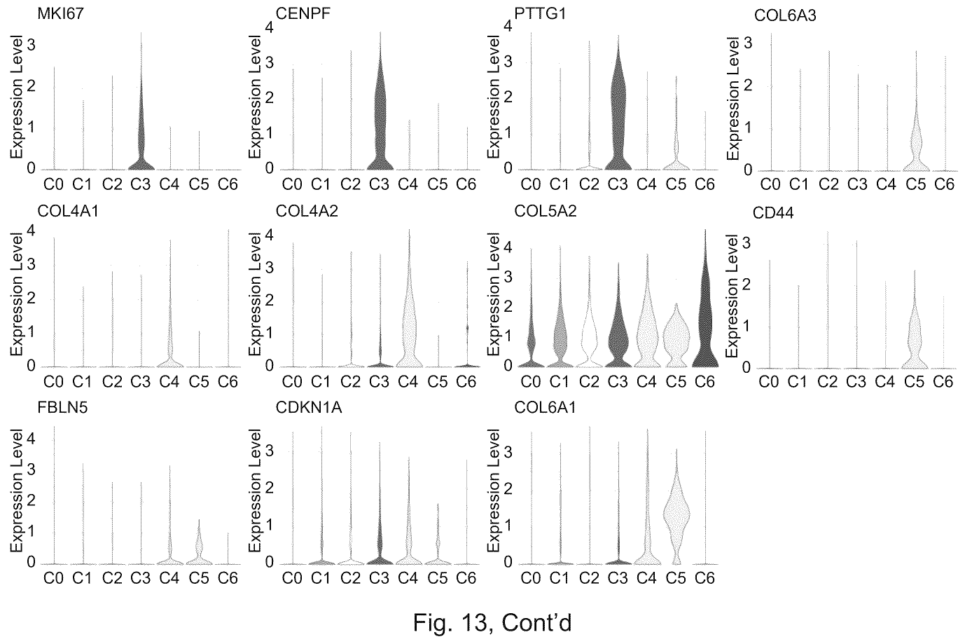

Increased differential expression of typical CEC markers, such as COL4A6 (Van den Bogerd et al., 2019. Transl Vis Sci Technol 8: 13), SLC4A11, ATP1A1, COL4A3, and CDH2 suggested that clusters C0 and C1 were composed of therapy-grade CECs (FIGS. 3B and 13). Further analysis by gene set enrichment analysis (GSEA) revealed that highly metabolically active cells comprised these clusters (FIG. 3C), with a distinct hallmark for oxidative phosphorylation in cluster C0 (FIG. 3D). These markers are also found in native functioning human CECs, suggesting these cells were therapy-grade CECs. Cluster C3 was composed of proliferating CECs with differential high expression of MKI67 (KI-67), CENPF, and PTTG1 (FIGS. 3B and 13). GSEA further confirmed enriched gene sets and significant hallmarks related to cell proliferation (FIGS. 3C, D).

Clusters C2 and C4 were composed of CECs with increased differential expression of senescence related genes. Namely, CDKN2A (p16), TAGLN, and MT2A (FIGS. 3B and 13) in cluster C2 and CDKN1A (p21) (Matthaei et al., 2012. Vis Sci 53: 6718-6727), CDKN2A (p16), and TAGLN in cluster C4 (FIGS. 3B and 13), suggesting these cells were transitioning towards an undesirable senescent and fibrotic phenotype. Cluster C4 had a high extracellular matrix production suggested by the differential high expression of COL4A1, COL4A2, COL5A1, and FBLN5 in cells that maintained the expression of endothelial markers such as SLC4A11, and COL4A3 (FIGS. 3B and 13). In contrast, cells in cluster C2 presented a low expression of CEC markers such as ALCAM, SLC4A11, CDH2, and ATP1A1 (FIGS. 3B and 13). GSEA revealed that both clusters C2 and C4 had a significant hallmark for epithelial to mesenchymal transition (FIG. 3D), confirming these cells were transitioning towards senescence and fibrosis. GSEA revealed that cluster C2 was enriched for genes related to alterations in the matrisome production, upregulation of p53 pathway, and upregulation on Rho GTPase pathway, which are known to regulate cellular senescence (Orgaz et al., 2014. Small GTPases 5: e983867; Rufini et al., 2013. Oncogene 32: 5129-5143) (FIG. 3C), and cluster C4 expression was enriched for genes related to matrisome, extracellular matrix organization and matrisome associated genes suggesting a remodeling of the extracellular matrix (FIG. 3C). Cluster C5 was composed of fibrotic cells differentially expressing the fibrosis-associated markers COL6A1, COL6A3, CD44, and ACTA2 (FIG. 3B). The cells in cluster C5 had diminished expression of ALCAM (CD166) and lacked SLC4A11 expression, two CEC markers. GSEA further suggested enriched expression of genes related to the matrisome and matrisome associated processes and increased signaling by G-coupled protein receptors and receptor tyrosine kinase (FIG. 3C), with a significant hallmark for epithelial to mesenchymal transition for cells in cluster C5 (FIG. 3D). Finally, Cluster C6 was composed of CECs with differential high expression of typical endothelial markers such as SLC4A11, COL4A3, and CDH2 (FIG. 3B) suggesting they were therapy-grade CECs. These cells differentially expressed higher GOLGA8A and GOLGA8B, suggesting a possible increase in secretory pathways. GSEA did not reveal significant upregulation of gene sets nor significant hallmarks compared to other clusters.

### Longer Culture Times Decrease Proliferation and Increase Transitioning to Senescence

Our scRNAseq data analysis revealed that at longer culture time points, namely confluency in passage 2, there was an increase in the number of cells transitioning to a senescent/fibrotic phenotype (clusters C2 and C4) and a decrease in the number of proliferative cells (cluster C3) compared to earlier time points (FIG. 3E). Interestingly, the number of cells in cluster C4 decreased over culture time (FIG. 3E) while cells in cluster C2 increased over time. This could be because the cells in C4 are early senescent cells and transition to later senescent cells in cluster C2. Regarding fibrotic CECs in cluster C5, these cells were detected as early as passage 1, but were also present in passage 2 at lower prevalence compared to passage 1. Finally, the prevalence ratio of therapy-grade CECs (cluster C0 and C1) was maintained over time points (FIG. 3E) showing the presence of therapy-grade CECs over all culture passages.

scRNAseq Sub-Clustering Analysis Revealed Two Distinct Transcriptomic Profiles of Proliferating Cells

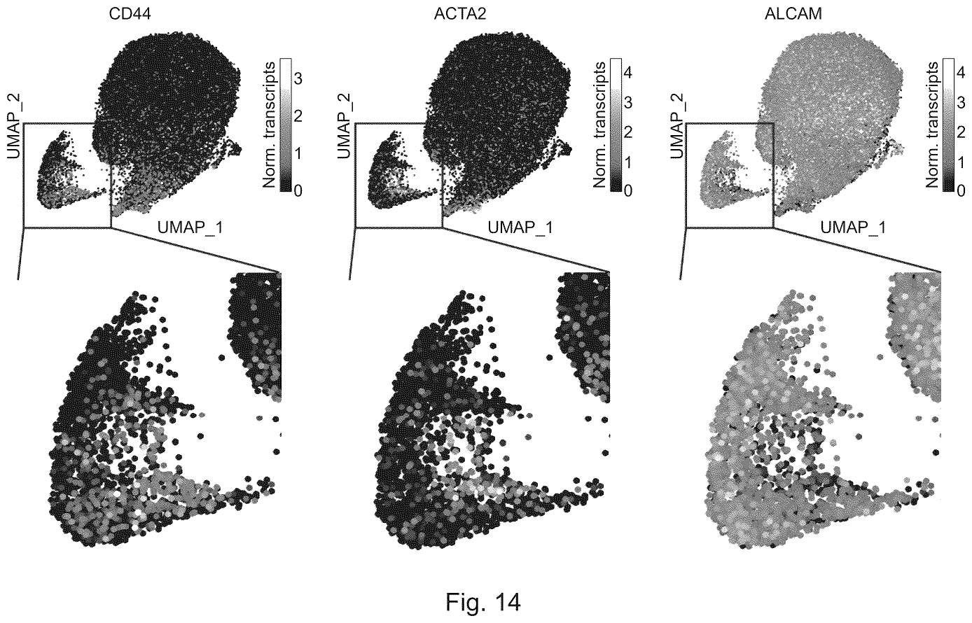

The CEC marker ALCAM, and the fibrotic markers CD44 and ACTA2 were heterogeneously expressed across different cells comprising proliferative cluster C3 (FIG. 14), suggesting it contained a sub-cluster of highly proliferative fibrotic CECs. Correlation and differential expression analysis revealed similarities between a sub-cluster of C3 with the fibrotic cells present in cluster C5 (FIG. 3F). This was confirmed by a re-clustering analysis of only clusters C3 and C5, which showed that a small subpopulation of cells originating from cluster C3 clustered together with the cells originating from cluster C5 (FIG. 3G). This finding confirms the presence of a subpopulation of highly proliferative fibrotic CECs within cluster C3.

### Primary Cultured CECs Resemble Native Human CECs

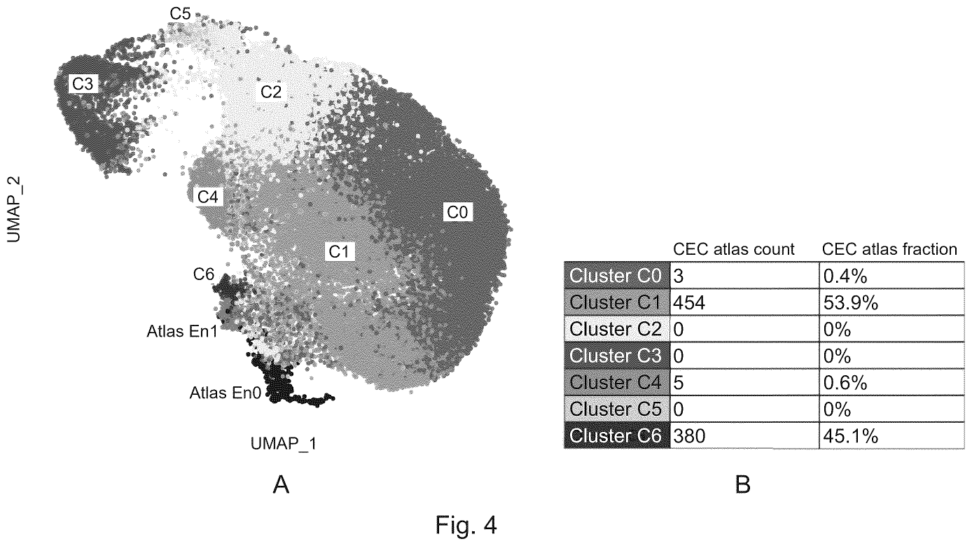

To assess how comparable primary cultured CECs are to native human CECs, we integrated the transcriptome of cells cultured to confluency in passages 0, 1, and 2 with a previously published cornea scRNAseq atlas (Catala et al., 2021. Sci Rep 11: 21727). To do so, the cluster information from both the CECs in the cornea cell atlas and this study were overlaid in 2D space (FIG. 4A). The clustering analysis revealed that the CEC clusters originating from the native human corneal endothelium (Atlas En0 and Atlas En1) clustered adjacent to primary CEC clusters C6, C1 and C0, suggesting these cell clusters share comparable transcriptomic profiles (FIG. 4A).

To further understand the similarity of native human CEC to primary cultured cells, we performed a cluster prediction analysis of the atlas CECs using the clustering analysis of cells at confluency. The prediction analysis revealed that 99.4% of the atlas CECs were associated to clusters C1 (53.9%), C6 (45.1%), and C0 (0.4%) (FIG. 4B), suggesting these clusters are similar to native human CECs and could be used for therapeutic purposes. Furthermore, our prediction analysis found that no native CECs were associated with the senescent and fibrotic clusters C2 and C5, or the proliferative cluster C3 (FIG. 4B).

Pseudo Time Reconstruction and Evaluation Reveal the Dynamics of CEC Profiles Arising from Primary Expansion

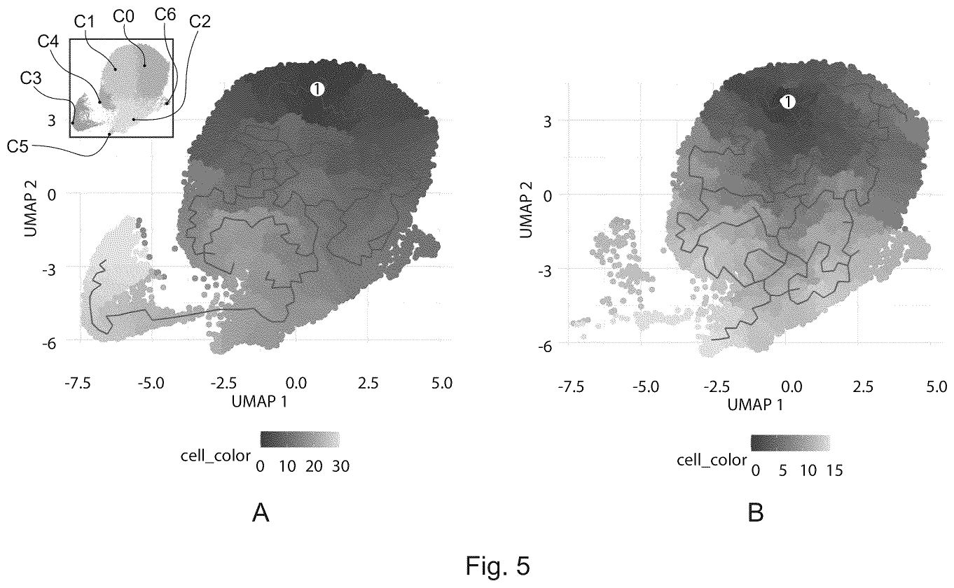

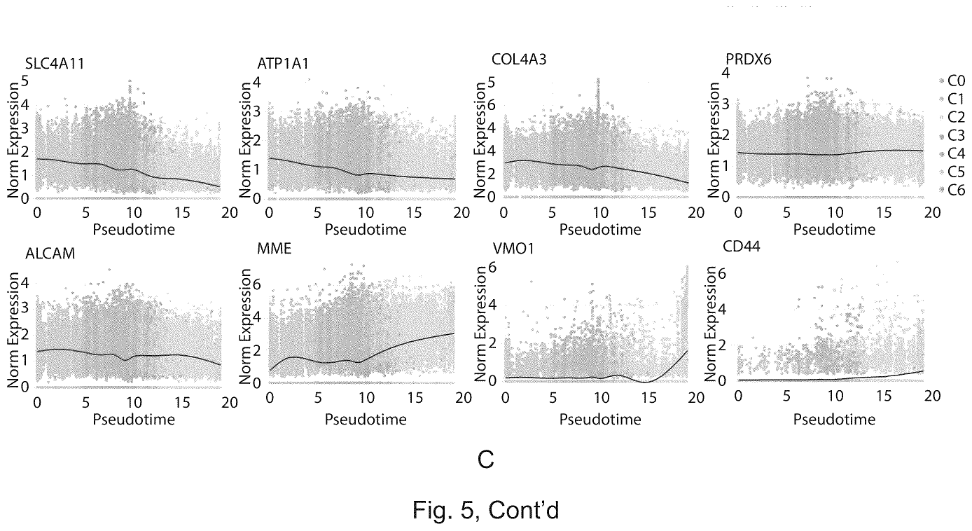

To assess how the cells transition between clusters over time, we performed a pseudo time reconstruction of the cells at confluency in passages 0, 1, and 2. Our first analysis revealed that the CECs originating from clusters C0, C1, and C6, transitioned into the senescent cells in cluster C2, and then became the fibrotic cells in cluster C5 (FIG. 5A). Interestingly, the last cluster the pseudo time trajectory identified were the proliferative cells in cluster C3. We hypothesize this is due to the presence of a side population of fibrotic proliferative cells within cluster C3 (FIG. 3F, G), which might interfere with the pseudo time trajectory analysis. To reduce such bias and identify gene trends over the pseudo time trajectory, we performed a pseudo time analysis of the confluent cells excluding cluster C3 (FIG. 5B). In line with the first analysis, the pseudo time trajectory revealed that the CECs from clusters C0, C1, and C6 transitioned into the transitioning senescent cells in cluster C2, and then the fibrotic cells in cluster C5 (FIG. 5B) showing that primary cultured CECs transition towards senescence and fibrosis over culture time.

The pseudo time reconstruction revealed that the expression of functional markers such as SLC4A11 and ATP1A1 was reduced over time, showing that senescent (cluster C2 and C4) and fibrotic (cluster C5) cells had a highly reduced expression of crucial functional markers (FIG. 5C). Moreover, pseudo time reconstruction also revealed reduced expression of COL4A3 and ALCAM over time (FIG. 5C). Interestingly, the expression of PRDX6, a known marker of CECs, remained constant and did not decrease over time in the senescent (C2 and C4) and fibrotic (C5) clusters (FIG. 5C). Besides crucial CEC markers, our analysis also revealed a significant increase of CD44 expression over time in clusters C2 and C5 CECs. Furthermore, the expression of MME (CD10) and VMO1 in the pseudo time analysis was increased in the senescent and fibrotic clusters C2 and C5 (FIG. 5C). These genes were also differentially expressed in lower quality clusters C2 and C5, respectively, and can be candidates to assess quality of primary cultured CECs.

scRNAseq Transcriptomic Profiles for Quality Assessment of Primary Cultured CEC Correlate with Protein Level Expression

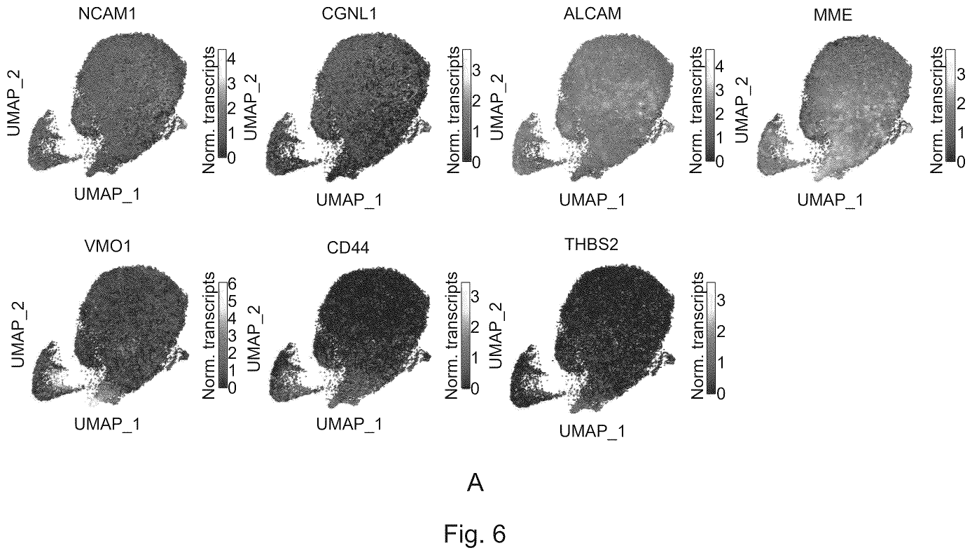

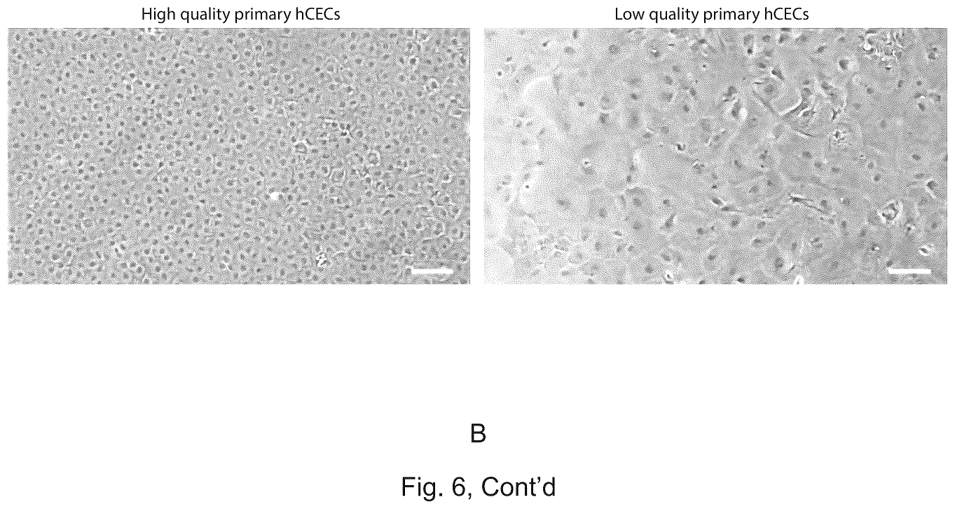

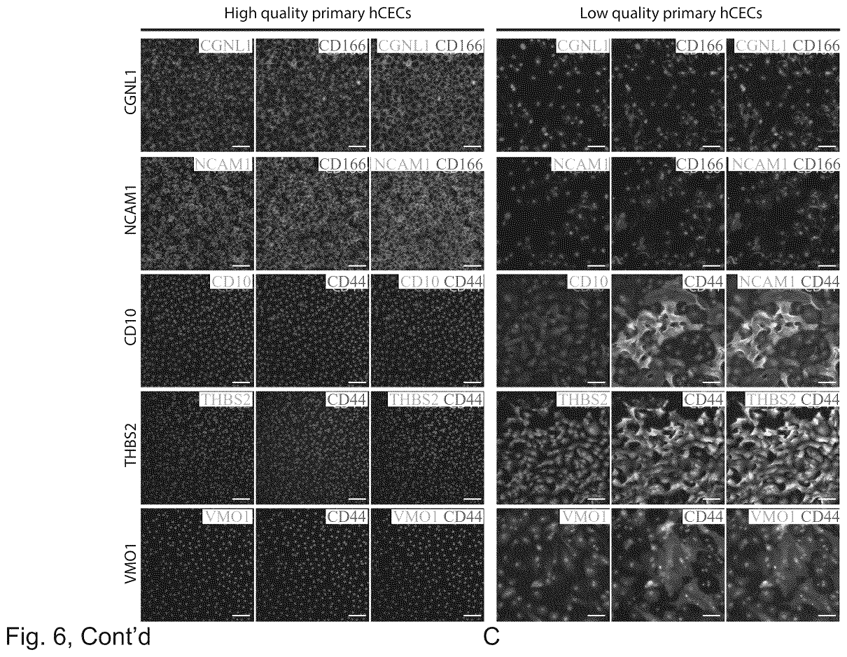

The differential gene expression across CEC clusters and the pseudo time reconstruction both showed that the quality of the primary cultured CEC could be evaluated with a specific set of markers to differentiate therapy-grade CEC (clusters C0, C1, and C6), from lower quality CECs transitioning towards a senescent or fibrotic phenotype (clusters C2 and C5). The expression of ALCAM (CD166), CGNL1 (cingulin-like protein 1), and NCAM1 (CD56) were higher in the clusters comprising therapy-grade CECs (clusters C0 and C1), and lower in the cells in clusters C2 and C5 (FIG. 6A). Additionally, the expression of CD44, MME (CD10), VMO1, and THBS2 were higher in the fibrotic cells in cluster C5 and lower in the cells in clusters C0, C1, and C6 (Figure CA), representing markers that could be used to exclude low quality CECs.

To confirm that the findings at the transcriptomic level were also present at the protein level, we assessed protein expression on primary CEC cultures with a characteristic hexagonal morphology associated with therapy-grade CECs (FIG. 6B, left panel) and on primary CEC cultures composed of cells with spindle shape morphology, a characteristic of cells undergoing an endothelial to mesenchymal transition, referred as low quality primary CECs (FIG. 6B, right panel). Immunofluorescence analysis confirmed that CGLN1, NCAM1 (CD56) and ALCAM (CD166) were exclusively expressed by good quality CECs and not expressed in the cultures containing CECs with an altered morphology (FIG. 6C). Furthermore, immunofluorescence analysis also confirmed that CD44, MME (CD10), THBS2, and VMO1 were exclusively expressed by low quality CECs and not expressed in high quality CEC cultures (FIG. 6C).

Further scRNAseq analysis not only revealed the (increased) differential expression of CD166, CGLN1 and CD56 in good quality, therapy-grade CECs, but also increased differential expression of amyloid beta precursor gene (APP) in such therapy-grade CECs. In addition, such further scRNAseq analysis revealed that apart from an increased differential expression of THBS2, VMO1, TPM2, CD44 and CD10 in inferior quality CECs transitioning to an undesired fibroblastic state (relative to good quality CECs), also S100 Calcium Binding Protein A10 (S100A10), thioredoxin reductase 1 (TXNRD1), potassium gated channel regulatory subunit E (KCNE4), Platelet Derived Growth Factor Receptor Alpha (PDGFRA or CD140a), and 4-Hydroxyphenylpyruvate Dioxygenase (HPPD) showed an increased differential expression in inferior quality CECs.

In this Example, we present a single-cell roadmap of human CECs in culture, revealing the diverse trajectories of individual cells. Our scRNAseq census of 42,200 primary cultured CECs revealed the presence of 7 clusters in therapeutically relevant time points, including therapy-grade CECs expressing SLC4A11, ALCAM, and COL4A3 (clusters C0, C1 and C6); highly proliferative CECs expressing MKT67 and CENPF (cluster C3); lower quality CECs entering senescence and EMT expressing CDKN1A, CDKN2A, and TAGLN (clusters C2 and C4); as well as fibrotic CECs expressing CD44, and ACTA2 (cluster C5). We assessed to which extent CECs in culture resemble native human CECs and analyzed how these CEC populations diverge over culture time, giving insights into the alterations arising during primary culture. Moreover, our transcriptomic profiling provides an array of combinatorial markers to differentiate therapy-grade CEC from cells undergoing senescence and EMT, thereby paving the way for improving culture protocols and guiding the selection of cells for therapy. The transcriptomic data we obtained will help better our understanding of the mechanisms involved in the alterations occurring during primary culture of CECs leading to a loss of function and phenotype.

Our analysis showed that proliferating sub-confluent CECs sequenced at day 2 and day 5 clustered together (cluster 3), with high differential expression of ribosomal-related genes. We hypothesize that this is most likely due to their necessary adaptation to in vitro culture conditions and the use of proliferation media, which biased the first clustering. These findings led us to separately explore the 37,158 cells at confluency in passages 0, 1, and 2. Our analysis revealed three clusters of therapy-grade CEC (clusters C0, C1, and C6) based on the high differential expression of functional CEC markers SLC4A11 and ATP1A1 and the CEC markers ALCAM, PRDX6, and COL4A3. These cells were the majority in all passages, comprising 70% of all the sequenced cells. GSEA revealed that cells in C0 and C1 were metabolically active with increased expression of genes related to oxidative phosphorylation, in line with the crucial activity and function of CECs (Deguchi et al., 2022. Sci Rep 12: 1-14).

The integration analysis of the dataset from this study with native CECs from a previously published cornea cell atlas (Catali et al., 2021. Sci Rep 11: 21727) revealed that native CECs clustered close to clusters C0, C1 and C6, suggesting the similarity of these cells. Cluster prediction analysis on native CECs revealed that these cells would group within clusters C1 (53.9%), C6 (45.1%), and C0 (0.4%). The low 0.4% prediction for cluster C0 is interesting because the primary cultured cells still expressed high levels of endothelial markers, namely SLC4A11, ATP1A1, ALCAM, and PRDX6. We hypothesize the low prediction might be due to a slight increase in ribosomal protein expression or a difference in the number of genes/cell which can be a technical sampling variation compared to clusters C1 and C6, which skews the cell clustering. Overall, our data shows that CECs in clusters C0, C1, and CC are good quality CECs and are a suitable source for therapy. And while our dataset shows that cells in clusters C1 and CC more closely resemble native human CECs than cells in cluster C0, it does not mean that cells in cluster C0 are unsuitable for therapy, but that they are distinguishable from native corneal endothelium.

Our scRNAseq analysis also revealed the presence of two clusters composed of senescent cells that were transitioning towards a mesenchymal phenotype (clusters C2 and C4). Our pseudo time reconstruction showed these cells were originating from the therapy-grade clusters C0, C1, and C6. This finding shows the transition to senescence of CECs during primary culture. Senescent cells in cluster C2 had reduced expression of key functional markers such as SLC4A11, ATP1A1, and ALCAM. This finding is in line with a recently published report that demonstrated a decrease of SLC4A11 in lower-quality primary CECs (Deguchi et al., 2022. Sci Rep 12: 1-14). The total number of senescent cells (clusters C2 and C4) increased over culture time, suggesting the CECs transitioned towards senescence over extended culture times. Cells comprising cluster C4 decreased over culture time, indicating that they either transition into senescent cells in cluster C2 or represent an end-point cluster, where cells tend to die over time. GSEA revealed that cells in cluster C2 had differential gene expression, specifically an increase in genes involved in the p53 and Rho GTPase pathways, suggesting these pathways might play a key role on the senescence and endothelial to mesenchymal transition of primary cultured CECs. P53 is a known senescence regulator (Mijit et al., 2020. Biomolecules 10: 420; Rufini et al., 2013. Oncogene 32: 5129-5143), and its inhibition could delay the cellular senescence in primary CECs. A study in 2013 revealed that the inhibition of p53 was associated with improved morphology and higher expression of CEC markers, namely collagen type 8, Na/K ATPase, and N-cadherin, in primary cultured CECs (Sha et al., 2013. Exp Eye Res 116: 36-46). Furthermore, our findings suggest that the inhibition of the Rho GTPase pathway can play a key role in delaying cellular senescence, further confirming that the use of Rho-associated protein kinase (ROCK) inhibitors such as Y-27632 might be a pivotal factor in the protocols for primary expansion of therapy-grade CECs. Indeed, previous studies have used Y-27632 for the primary expansion of CECs (Kinoshita et al., 2018. N Engl J Med 378: 995-1003; Okumura et al., 2009. Investig Ophthalmol Vis Sci 50: 3680-3687; Parekh et al., 2021. Acta Ophthalmol 99: e512-e522; Peh et al., 2015. Sci Rep 5: 1-10). Based on these findings, we therefore recommend the use of ROCK inhibitors during the primary expansion of CECs.

Our scRNAseq analysis revealed that a cluster of CECs (cluster C5) expressing characteristic fibrotic markers ACTA2 and CD44 originated from the senescent cells at clusters C2, suggesting a transition from senescence to endothelial to mesenchymal transition phenotype. While fibrotic markers were found in later time points, we did observe that cells comprising cluster C5 appeared as early as passage 1, but were then reduced by passage 2. This finding could be due to the sequencing sampling limitation of 10,000 cells per sample, making it highly possible that this small fibrotic cell population was not sequenced from a culture of hundreds of thousands of cells. Our second hypothesis is that after passaging, the fibrotic cells could not successfully adhere, causing a reduction of this cell population and enriching for good quality cells.

Our findings showed that extended culture times decreased the proliferation potential of CECs, shown by a reduction in the number of cells in the proliferation cluster C3 across passages. Moreover, we detected the presence of a subpopulation of undesired proliferative fibrotic cells that could potentially overgrow the culture of CECs over extended culture periods. In our view, these results show that with the current protocols, culturing CECs further than passage 2 is incompatible with their therapeutic use, a recommendation in line with previous studies that suggested passage 2 as the threshold time point to assure the therapeutic suitability of primary cultured CECs (Frausto et al., 2016. Cell Transplant 25: 1159-1176; Frausto et al., 2020 Sci Rep 10: 7402; Peh et al., 2019. Sci Rep 9: 6087).

Selecting and assessing the quality of the primary cultured CECs are a crucial aspect to ensure a safe and efficacious therapy. Based on our differential expression analysis and pseudo time reconstruction, we show that therapy-grade CEC should be identified by the expression of CD166 and CD56 membrane proteins together with CGNL1, a membrane-associated protein to cellular tight junctions; lack of expression of altered extracellular matrix, namely VMO1 and THBS2; and lack of expression of membrane proteins CD44 and CD10. While the membrane protein markers suggested by our analysis would allow sorting for therapy-grade CECs, we believe is equally important to characterize CEC culture quality based on expression of other fundamental proteins such as aberrant extracellular matrix production. Future studies are required to understand how the expression of these markers correlate to therapeutic success. Similar to our suggestion to analyze markers for therapy-grade CECs (CD166⁺, NCAM1⁺, CGNL1⁺, CD44⁻, CD10⁻, VMO1⁻, and THBS2⁻), Kinoshita and colleagues proposed the combinatorial marker expression referred as the E-ratio (CD166⁺, CD44⁻, CD133⁻, CD24⁻, and CD105⁻) to assess for therapy-grade CECs. We and they both detected increased CD166 expression in therapy-grade CECs and CD44 exclusively expressed in lower quality senescent and transitioning CECs. By contrast, our analysis revealed that CD24 and ENG (CD105) were heterogeneously and minimally expressed across clusters in some CECs, and we did not detect expression of PROM1 (CD133) in any cluster. These differences might be due to the lack of correlation between transcript and protein detection. Future studies analyzing such differences can shed light on the suitability of markers to assess or enrich for therapy-grade CECs.

While primary cultured CECs have been traditionally assessed as bulk entities without accounting for their heterogeneity, our study analyses them at the single-cell level over five culture time points in three different passages. Our study provides significant information to help understand the changes arising from the culture of human CECs, portraying their cellular heterogeneity as well as characterizing their variability over extended culture times. These results provide a pivotal dataset that can help identify and characterize the undesired cell populations arising from primary culture in the attempt to improve current protocols. Our results also show the importance of supplementing media for CEC expansion with ROCK inhibitors to reduce cellular senescence. Furthermore, based on the results reported in this study, we propose a combination of markers to assess the quality of primary cultured CECs. Overall, this transcriptomic cell analysis offers a baseline for future studies with the aim of improving CEC-based therapies.

1. A method for determining the quality of cultured corneal endothelial cells (CECs) comprising measuring in or on a cultured primary CEC a level of expression of the genes Thrombospondin-2 (THBS2) and/or Vitelline Membrane Outer Layer 1 Homolog (VMO1), and determining that the quality of said cultured primary CEC is non-therapy-grade if said measured level of expression corresponds to a THBS2+ and/or VMO1+ expression profile.

2. The method of claim 1, wherein said level of expression is measured as the absence or presence of the protein expression product of said genes, and wherein it is determined that the quality of said cultured CEC is non-therapy-grade if said protein expression product is detected, preferably wherein absence or presence of the protein expression product is measured by using fluorescently labeled antibodies.

3. The method of claim 1, further comprising measuring in or on said cultured CEC a level of expression of the gene Cingulin-like 1 (CGNL1), and determining that the quality of said cultured CEC is non-therapy-grade if said measured level of expression corresponds to a VMO1+, THBS2+, CGNL1− expression profile.

4. The method of claim 1, further comprising measuring in or on said cultured CEC a level of expression of at least one, preferably all, of CD166, CD56, CD44 and CD10, and determining that the quality of said cultured CEC is non-therapy-grade if said measured level of expression corresponds to a CGNL1⁻, VMO1+, THBS2+, CD166⁻, CD56⁻, CD44+, and/or CD10+ expression profile, or determining that the quality of said cultured CEC is therapy-grade if said measured level of expression corresponds to a CGNL1+, VMO1⁻, THBS2⁻, CD166+, CD56+, CD44⁻, and CD10⁻ expression profile.

5. The method of claim 1, further comprising measuring in or on said cultured CEC a level of expression of at least one gene selected from Amyloid Beta Precursor Protein (APP), S100 Calcium Binding Protein A10 (S100A10), Thioredoxin Reductase 1 (TXNRD1), Tropomyosin 2 (TPM2), Potassium Voltage-Gated Channel Subfamily E Regulatory Subunit 4 (KCNE4), Platelet Derived Growth Factor Receptor Alpha (PDGFRA), and 4-Hydroxyphenylpyruvate Dioxygenase (HPPD), and determining that the quality of said cultured CEC is non-therapy-grade if said measured level of expression corresponds to a APP⁻, S100A10±, TXNRD1+, TPM2+, KCNE4+, PDGFRA±, and/or HPPD expression profile, or determining that the quality of said cultured CEC is therapy-grade if said measured level of expression corresponds to a CGNL1+, VMO1⁻, THBS2⁻, CD166±, CD56±, CD44⁻, CD10⁻ APR′, S100A10⁻, TXNRD1⁻, TPM2⁻, KCNE4⁻, PDGFRA⁻, and/or HPPD⁻ expression profile.

6. The method of claim 1, wherein said cultured CEC is of human origin.

7. The method of claim 1, wherein said CEC is an individual CEC, more preferably wherein the level of expression is determined in or on a single cell.

8. The method of claim 1, wherein said measurement is performed on a cultured CEC at confluency in passages 0, 1, or 2.

9. The method of claim 1, wherein said measurement is performed on a cultured CEC cultured in the presence of a ROCK kinase inhibitor.

10. The method of claim 1, wherein the level of expression is measured by immunofluorescence techniques, preferably using fluorescently labeled antibodies.

11. A method of excluding or selecting a culture of CECs for medical use, the method comprising:
a) providing an in vitro culture of CECs, preferably cultured primary CECs or stem-cell-derived CECs, more preferably human CECs,
b) performing the method of claim 1 on at least a part of said culture, and
c) excluding said culture of CECs as a culture of CECs unsuitable for medical use when more than 10-15% of the cell population in said culture of CECs is of non-therapy-grade quality or when less than 85-90% of the cell population in said culture of CECs is of therapy-grade quality, or
selecting said culture of CECs as a culture of CECs for medical use when the cell population in said culture of CECs comprises CECs of which less than 10-15% is of non-therapy-grade quality or when more than 85-90% of the cell population in said culture of CECs is of therapy-grade quality.

12. A method of producing a culture of CECs for medical use, comprising performing the method of claim 11, wherein step c) comprises selecting said culture of CECs as a culture of CECs for medical use, and wherein said method further comprises the step of providing a culture of CECs by:
harvesting the in vitro culture of CECs as culture of CECs for medical use when the incidence of therapy-grade quality CECs in said culture is at a desired level; or
separating therapy-grade quality CECs from said in vitro culture of CECs and collecting the thus separated cells as a culture of CECs for medical use; or
removing non-therapy-grade quality CECs from said in vitro culture of CECs and collecting the remainder of said culture as a culture of CECs for medical use.

13. The method of claim 11, wherein step a) comprises cultivating said CECs as a monolayer, optionally dissociating said monolayer, and providing CECs in said optionally dissociated monolayer with fresh tissue culture reagent to produce a subsequent passage, preferably wherein said cultivation occurs in the presence of a ROCK kinase inhibitor and/or a p53 inhibitor, and/or preferably wherein the CECs are cultivated in or on a substrate or scaffold.

14. A culture of CECs, preferably in the form of a monolayer, wherein the cell population in said culture of CECs comprises CECs of which at least 85-90% is of therapy-grade quality, and of which at least 85-90% comprises a VMO1- and/or THBS2-expression profile,
preferably wherein said culture is selected or produced by the method of claim 11, and
wherein said culture optionally comprises antibodies against a protein expression product of at least one gene selected from CGNL1, VMO1, THBS2, APP, S100A10, TXNRD1, TPM2, KCNE4, PDGFRA, and HPPD, preferably selected from CGNL1 and APP, most preferably CGNL1.

15. A tissue engineered graft material comprising a culture of CECs according to claim 14.

16. A method for treating a patient suffering from corneal endothelial dysfunction comprising administering to said patient the culture of CECs of claim 14, or a tissue engineered graft material comprising said culture of CECs.

17. A computer-implemented method for determining the quality of cultured CECs comprising steps of:
a) receiving data representing a level of expression of THBS2 and/or VMO1, preferably further including CGNL1, CD166, CD56, CD44 and CD10, said level of expression having been measured in or on at least one cultured CEC, and
b) determining the quality of a cultured CEC based on comparing said data received under a) with reference data of the level of expression in a therapy-grade and/or non-therapy-grade reference CEC, wherein a CD166±, CD56±, CGNL1+, CD44⁻, CD10⁻, VMO1⁻, and THBS2⁻ expression profile is indicative of the therapy-grade quality of said cultured CEC, and wherein a VMO1±, THBS2+, CGNL1⁻, CD166⁻, CD56⁻, CD44±, and/or CD10±, expression profile is indicative of the non-therapy-grade quality of said cultured CEC
wherein step b) is preferably performed on a computer using an algorithm wherein the data received in step a) are compared with reference data, preferably wherein said reference values are stored on an accessible memory, and preferably wherein said algorithm can receive the data of step a).

18. A data processing system comprising a processor adapted to perform the steps of the computer-implemented method of claim 17, or a computer program product comprising instructions which, when the program is executed by a computer, cause the computer to carry out the computer-implemented method of claim 17, optionally comprising a machine learning data processing model.
